SEQUENCE LISTING (77 sequences)

SEQ ID NO: 1 - DNA, 1851 bp, Homo sapien
tcatcaccattgccagcagcggcaccgttagtcaggttttctgggaatcccacatgagtacttccgtgttcttcattcttcttcaatagccataaatcttctagctctggctggctgttttcacttcctttaagcctttgtgactcttcctctgatgtcagctttaagtcttgttctggattgctgttttcagaagagatttttaacatctgtttttctttgtagtcagaaagtaactggcaaattacatgatgatgactagaaacagcatactctctggccgtctttccagatcttgagaagatacatcaacattttgctcaagtagagggctgactatacttgctgatccacaacatacagcaagtatgagagcagttcttccatatctatccagcgcatttaaattcgcttttttcttgattaaaaatttcaccacttgctgtttttgctcatgtataccaagtagcagtggtgtgaggccatgcttgttttttgattcgatatcagcaccgtataagagcagtgctttggccattaatttatcttcattgtagacagcatagtgtagagtggtatttccatactcatctggaatatttggatcagtgccatgttccagcaacattaacgcacattcatcttcctggcattgtacggcctttgtcagagctgtcctctttttgttgtcaaggacattaagttgacatcgtctgtccagcacgagttttactacttctgaattcccattggcagaggccagatgtagagcagtcctcttttgcttgtccctcttgttcacatccgtgtccctgagcatgacgatgagatcctttctggggactttaccccaccaggcagctctgtggagcttgtccagatcttctccatggacgtggtacctgggatccatgaaggcgctgtcatcgtagtctccccaagcgaccacgttgctcttgccgctcccctgcagcaggggaagcagtggcagcaccacttgcacctcttgctcccaagcgtcttcacagaggagtcgttgtggtctccagaagtgcccacgttgctcttgccgctccccctgtccatccagggaggaagaaatgcaggaaatgaaagatgcatgcacgatggtatactcctcagccatcaaacttctggacagcaggtcacttccagcaaggtggagaaagctgtccacccacagaggatgagatccagaaaccacaatatccattcacaaacaaacacttttcagccagacacaggtactgaaatcatgtcatctgcggcaacatggtggaacctacccaatcacacatcaagagatgaagacactgcagtatatctgcacaacgtaatactcttcatccataacaaaataatataattttcctctggagccatatggatgaactatgaaggaagaactccccgaagaagccagtcgcagagaagccacactgaagctctgtcctcagccatcagcgccacggacaggartgtgtttcttccccagtgatgcagcctcaagttatcccgaagctgccgcagcacacggtggctcctgagaaacaccccagctcttccggtctaacacaggcaagtcaataaatgtgataatcacataaacagaattaaaagcaaagtcacataagcatctcaacagacacagaaaaggcatttgacaaaatccagcatccttgtatttattgttgcagttctcagaggaaatgcttctaacttttccccatttagtattatgttggctgtgggcttgtcataggtggtttttattactttaaggtatgtcccttctatgcctgttttgctgagggttttaattctcgtgcc

SEQ ID NO: 2 - protein, 329 aa, Homo sapien
MDIVVSGSHPLWVDSFLHLAGSDLLSRSLMAEEYTIVHASFISCISSSLDGQGERQEQRGHFWRPQRLLCEDAWEQEVQVVLPLLPLLQGSGKSNVVAWGDYDDSAFMDPRYHVHGEDLDKLHRAAWWGKVPRKDLIVMLRDTDVNKRDKQKRTALHLASANGNSEVVKLVLDRRCQLNVLDNKKRTALTKAVQCQEDECALMLLEHGTDPNIPDEYGNTTLHYAVYNEDKLMAKALLLYGADIESKNKHGLTPLLLGIHEQKQQVVKFLIKKKANLNALDRYGRTALILAVCCGSASIVSPLLEQNVDVSSQDLERRPESMLFLVIIM

SEQ ID NO: 3 - DNA, 1852 bp, Homo sapiens
ggcacgagaattaaaaccctcagcaaaacaggcatagaagggacataccttaaagtaataaaaaccacctatgacaagcccacagccaacataatactaaatggggaaaagttagaagcatttcctctgagaactgcaacaataaatacaaggatgctggattttgtcaaatgccttttctgtgtctgttgagatgcttatgtgactttgcttttaattctgtttatgtgattatcacatttattgacttgcctgtgttagaccggaagagctggggtgtttctcaggagccaccgtgtgctgcggcagcttcgggataacttgaggctgcatcactggggaagaaacacaytcctgtccgtggcgctgatggctgaggacagagcttcagtgtggcttctctgcgactggcttcttcggggagttcttccttcatagttcatccatatggctccagaggaaaattatattattttgttatggatgaagagtattacgttgtgcagatatactgcagtgtcttcatctcttgatgtgtgattgggtaggttccaccatgttgccgcagatgacatgatttcagtacctgtgtctggctgaaaagtgtttgtttgtgaatggatattgtggtttctggatctcatcctctgtgggtggacagctttctccaccttgctggaagtgacctgctgtccagaagtttgatggctgaggagtataccatcgtgcatgcatctttcatttcctgcatttcttcctccctggatggacagggggagcggcaagagcaacgtgggcacttctggagaccacaacgactcctctgtgaagacgcttgggagcaagaggtgcaagtggtgctgccactgcttcccctgctgcagggggagcggcaagagcaacgtggtcgcttggggagactacgatgacagcgccttcatggatcccaggtaccacgtccatggagaagatctggacaagctccacagagctgcctggtggggtaaagtccccagaaaggatctcatcgtcatgctcagggacacggatgtgaacaagagggacaagcaaaagaggactgctctacatctggcctctgccaatgggaattcagaagtagtaaaactcgtgctggacagacgatgtcaacttaatgtccttgacaacaaaaagaggacagctctgacaaaggccgtacaatgccaggaagatgaatgtgcgttaatgttgctggaacatggcactgatccaaatattccagatgagtatggaaataccactctacactatgctgtctacaatgaagataaattaatggccaaagcactgctcttatacggtgctgatatcgaatcaaaaaacaagcatggcctcacaccactgctacttggtatacatgagcaaaaacagcaagtggtgaaatttttaatcaagaaaaaagcgaatttaaatgcgctggatagatatggaagaactgctctcatacttgctgtatgttgtggatcagcaagtatagtcagccctctacttgagcaaaatgttgatgtatcttctcaagatctggaaagacggccagagagtatgctgtttctagtcatcatcatgtaatttgccagttactttctgactacaaagaaaaacagatgttaaaaatctcttctgaaaacagcaatccagaacaagacttaaagctgacatcagaggaagagtcacaaaggcttaaaggaagtgaaaacagccagccagagctagaagatttatggctattgaagaagaatgaagaacacggaagtactcatgtgggattcccagaaaacctgactaacggtgccgctgctggcaatggtgatga

SEQ ID NO: 4 - protein, 292 aa, Homo sapiens
MHLSFPAFLPPWMDRGSGKSNVGTSGDHNDSSVKTLGSKRCKWCCHCFPCCRGSGKSNVVAWGDYDDSAFMDPRYHVHGEDLDKLHRAAWWGKVPRKDLIVMLRDTDVNKRDKQKRTALHLASANGNSEVVKLVLDRRCQLNVLDNKKRTALTKAVQCQEDECALMLLEHGTDPNIPDEYGNTTLHYAVYNEDKLMAKALLLYGADIESKNKHGLTPLLLGIHEQKQQVVKFLIKKKANLNALDRYGRTALILAVCCGSASIVSPLLEQNVDVSSQDLERRPESMLFLVIIM

SEQ ID NO: 5 - DNA, 1155 bp, Homo sapien
atggtggttgaggttgattccatgccggctgcctcttctgtgaagaagccatttggtctcaggagcaagatgggcaagtggtgctgccgttgcttcccctgctgcagggagagcggcaagagcaacgtgggcacttctggagaccacgacgactctgctatgaagacactcaggagcaagatgggcaagtggtgccgccactgcttcccctgctgcagggggagtggcaagagcaacgtgggcgcttctggagaccacgacgactctgctatgaagacactcaggaacaagatgggcaagtggtgctgccactgcttcccctgctgcagggggagcggcaagagcaaggtgggcgcttggggagactacgatgacagtgccttcatggagcccaggtaccacgtccgtggagaagatctggacaagctccacagagctgcctggtggggtaaagtccccagaaaggatctcatcgtcatgctcagggacactgacgtgaacaagaaggacaagcaaaagaggactgctctacatctggcctctgccaatgggaattcagaagtagtaaaactcctgctggacagacgatgtcaacttaatgtccttgacaacaaaaagaggacagctctgataaaggccgtacaatgccaggaagatgaatgtgcgttaatgttgctggaacatggcactgatccaaatattccagatgagtatggaaataccactctgcactacgctatctataatgaagataaattaatggccaaagcactgctcttatatggtgctgatatcgaatcaaaaaacaagcatggcctcacaccactgttacttggtgtacatgagcaaaaacagcaagtcgtgaaatttttaatcaagaaaaaagcgaatttaaatgcactggatagatatggaaggactgctctcatacttgctgtatgttgtggatcagcaagtatagtcagccttctacttgagcaaaatattgatgtatcttctcaagatctatctggacagacggccagagagtatgctgtttctagtcatcatcatgtaatttgccagttactttctgactacaaagaaaaacagatgctaaaaatctcttctgaaaacagcaatccagaaaatgtctcaagaaccagaaataaataa

SEQ ID NO: 6 - DNA, 2000 bp, Homo sapien
atggtggttgaggttgattccatgccggctgcctcttctgtgaagaagccatttggtctcaggagcaagatgggcaagtggtgctgccgttgcttcccctgctgcagggagagcggcaagagcaacgtgggcacttctggagaccacgacgactctgctatgaagacactcaggagcaagatgggcaagtggtgccgccactgcttcccctgctgcagggggagtggcaagagcaacgtgggcgcttctggagaccacgacgactctgctatgaagacactcaggaacaagatgggcaagtggtgctgccactgcttcccctgctgcagggggagcggcaagagcaaggtgggcgcttggggagactacgatgacagtgccttcatggagcccaggtaccacgtccgtggagaagatctggacaagctccacagagctgcctggtggggtaaagtccccagaaaggatctcatcgtcatgctcagggacactgacgtgaacaagaaggacaagcaaaagaggactgctctacatctggcctctgccaatgggaattcagaagtagtaaaactcctgctggacagacgatgtcaacttaatgtccttgacaacaaaaagaggacagctctgataaaggccgtacaatgccaggaagatgaatgtgcgttaatgttgctggaacatggcactgatccaaatattccagatgagtatggaaataccactctgcactacgctatctataatgaagataaattaatggccaaagcactgctcttatatggtgctgatatcgaatcaaaaaacaagcatggcctcacaccactgttacttggtgtacatgagcaaaaacagcaagtcgtgaaatttttaatcaagaaaaaagcgaatttaaatgcactggatagatatggaaggactgctctcatacttgctgtatgttgtggatcagcaagtatagtcagccttctacttgagcaaaatattgatgtatcttctcaagatctatctggacagacggccagagagtatgctgtttctagtcatcatcatgtaatttgccagttactttctgactacaaagaaaaacagatgctaaaaatctcttctgaaaacagcaatccagaacaagacttaaagctgacatcagaggaagagtcacaaaggttcaaaggcagtgaaaatagccagccagagaaaatgtctcaagaaccagaaataaataaggatggtgatagagaggttgaagaagaaatgaagaagcatgaaagtaataatgtgggattactagaaaacctgactaatggtgtcactgctggcaatggtgataatggattaattcctcaaaggaagagcagaacacctgaaaatcagcaatttcctgacaacgaaagtgaagagtatcacagaatttgcgaattagtttctgactacaaagaaaaacagatgccaaaatactcttctgaaaacagcaacccagaacaagacttaaagctgacatcagaggaagagtcacaaaggcttgagggcagtgaaaatggccagccagagctagaaaattttatggctatcgaagaaatgaagaagcacggaagtactcatgtcggattcccagaaaacctgactaatggtgccactgctggcaatggtgatgatggattaattcctccaaggaagagcagaacacctgaaagccagcaatttcctgacactgagaatgaagagtatcacagtgacgaacaaaatgatactcagaagcaattttgtgaagaacagaacactggaatattacacgatgagattctgattcatgaagaaaagcagatagaagtggttgaaaaaatgaattctgagctttctcttagttgtaagaaagaaaaagacatcttgcatgaaaatagtacgttgcgggaagaaattgccatgctaagactggagctagacacaatgaaacatcagagccagctaaaaaaaaaaaaaaaaaaaaaaaaaaaaaaaaa

SEQ ID NO: 7 - DNA, 2040 bp, Homo sapien
atggtggttgaggttgattccatgccggctgcctcttctgtgaagaagccatttggtctcaggagcaagatgggcaagtggtgctgccgttgcttcccctgctgcagggagagcggcaagagcaacgtgggcacttctggagaccacgacgactctgctatgaagacactcaggagcaagatgggcaagtggtgccgccactgcttcccctgctgcagggggagtggcaagagcaacgtgggcgcttctggagaccacgacgactctgctatgaagacactcaggaacaagatgggcaagtggtgctgccactgcttcccctgctgcagggggagcggcaagagcaaggtgggcgcttggggagactacgatgacagtgccttcatggagcccaggtaccacgtccgtggagaagatctggacaagctccacagagctgcctggtggggtaaagtccccagaaaggatctcatcgtcatgctcagggacactgacgtgaacaagaaggacaagcaaaagaggactgctctacatctggcctctgccaatgggaattcagaagtagtaaaactcctgctggacagacgatgtcaacttaatgtccttgacaacaaaaagaggacagctctgataaaggccgtacaatgccaggaagatgaatgtgcgttaatgttgctggaacatggcactgatccaaatattccagatgagtatggaaataccactctgcactacgctatctataatgaagataaattaatggccaaagcactgctcttatatggtgctgatatcgaatcaaaaaacaagcatggcctcacaccactgttacttggtgtacatgagcaaaaacagcaagtcgtgaaatttttaatcaagaaaaaagcgaatttaaatgcactggatagatatggaaggactgctctcatacttgctgtatgttgtggatcagcaagtatagtcagccttctacttgagcaaaatattgatgtatcttctcaagatctatctggacagacggccagagagtatgctgtttctagtcatcatcatgtaatttgccagttactttctgactacaaagaaaaacagatgctaaaaatctcttctgaaaacagcaatccagaacaagacttaaagctgacatcagaggaagagtcacaaaggttcaaaggcagtgaaaatagccagccagagaaaatgtctcaagaaccagaaataaataaggatggtgatagagaggttgaagaagaaatgaagaagcatgaaagtaataatgtgggattactagaaaacctgactaatggtgtcactgctggcaatggtgataatggattaattcctcaaaggaagagcagaacacctgaaaatcagcaatttcctgacaacgaaagtgaagagtatcacagaatttgcgaattagtttctgactacaaagaaaaacagatgccaaaatactcttctgaaaacagcaacccagaacaagacttaaagctgacatcagaggaagagtcacaaaggcttgagggcagtgaaaatggccagccagagaaaagatctcaagaaccagaaataaataaggatggtgatagagagctagaaaattttatggctatcgaagaaatgaagaagcacggaagtactcatgtcggattcccagaaaacctgactaatggtgccactgctggcaatggtgatgatggattaattcctccaaggaagagcagaacacctgaaagccagcaatttcctgacactgagaatgaagagtatcacagtgacgaacaaaatgatactcagaagcaattttgtgaagaacagaacactggaatattacacgatgagattctgattcatgaagaaaagcagatagaagtggttgaaaaaatgaattctgagctttctcttagttgtaagaaagaaaaagacatcttgcatgaaaatagtacgttgcgggaagaaattgccatgctaagactggagctagacacaatgaaacatcagagccagctaaaaaaaaaaaaaaaaaaaaaaaaaaaa

SEQ ID NO: 8 - protein, 384 aa, Homo sapien
MVVEVDSMPAASSVKKPFGLRSKMGKWCCRCFPCCRESGKSNVGTSGDHDDSAMKTLRSKMGKWCRHCFPCCRGSGKSNVGASGDHDDSAMKTLRNKMGKWCCHCFPCCRGSGKSKVGAWGDYDDSAFMEPRYHVRGEDLDKLHRAAWWGKVPRKDLIVMLRDTDVNKKDKQKRTALHLASANGNSEVVKLLLDRRCQLNVLDNKKRTALIKAVQCQEDECALMLLEHGTDPNIPDEYGNTTLHYAIYNEDKLMAKALLLYGADIESKNKHGLTPLLLGVHEQKQQVVKFLIKKKANLNALDRYGRTALILAVCCGSASIVSLLLEQNIDVSSQDLSGQTAREYAVSSHHHVICQLLSDYKEKQMLKISSENSNPENVSRTRNK

SEQ ID NO: 9 - protein, 656 aa, Homo sapien
MVVEVDSMPAASSVKKPFGLRSKMGKWCCRCFPCCRESGKSNVGTSGDHDDSAMKTLRSKMGKWCRHCFPCCRGSGKSNVGASGDHDDSAMKTLRNKMGKWCCHCFPCCRGSGKSKVGAWGDYDDSAFMEPRYHVRGEDLDKLHRAAWWGKVPRKDLIVMLRDTDVNKKDKQKRTALHLASANGNSEVVKLLLDRRCQLNVLDNKKRTALIKAVQCQEDECALMLLEHGTDPNIPDEYGNTTLHYAIYNEDKLMAKALLLYGADIESKNKHGLTPLLLGVHEQKQQVVKFLIKKKANLNALDRYGRTALILAVCCGSASIVSLLLEQNIDVSSQDLSGQTAREYAVSSHHHVICQLLSDYKEKQMLKISSENSNPEQDLKLTSEEESQRFKGSENSQPEKMSQEPEINKDGDREVEEEMKKHESNNVGLLENLTNGVTAGNGDNGLIPQRKSRTPENQQFPDNESEEYHRICELVSDYKEKQMPKYSSENSNPEQDLKLTSEEESQRLEGSENGQPELENFMAIEEMKKHGSTHVGFPENLTNGATAGNGDDGLIPPRKSRTPESQQFPDTENEEYHSDEQNDTQKQFCEEQNTGILHDEILIHEEKQIEVVEKMNSELSLSCKKEKDILHENSTLREEIAMLRLELDTMKHQSQL

SEQ ID NO: 10 - protein, 671 aa, Homo sapien
MVVEVDSMPAASSVKKPFGLRSKMGKWCCRCFPCCRESGKSNVGTSGDHDDSAMKTLRSKMGKWCRHCFPCCRGSGKSNVGASGDHDDSAMKTLRNKMGKWCCHCFPCCRGSGKSKVGAWGDYDDSAFMEPRYHVRGEDLDKLHRAAWWGKVPRKDLIVMLRDTDVNKKDKQKRTALHLASANGNSEVVKLLLDRRCQLNVLDNKKRTALIKAVQCQEDECALMLLEHGTDPNIPDEYGNTTLHYAIYNEDKLMAKALLLYGADIESKNKHGLTPLLLGVHEQKQQVVKFLIKKKANLNALDRYGRTALILAVCCGSASIVSLLLEQNIDVSSQDLSGQTAREYAVSSHHHVICQLLSDYKEKQMLKISSENSNPEQDLKLTSEEESQRFKGSENSQPEKMSQEPEINKDGDREVEEEMKKHESNNVGLLENLTNGVTAGNGDNGLIPQRKSRTPENQQFPDNESEEYHRICELVSDYKEKQMPKYSSENSNPEQDLKLTSEEESQRLEGSENGQPEKRSQEPEINKDGDRELENFMAIEEMKKHGSTHVGFPENLTNGATAGNGDDGLIPPRKSRTPESQQFPDTENEEYHSDEQNDTQKQFCEEQNTGILHDEILIHEEKQIEVVEKMNSELSLSCKKEKDILHENSTLREEIAMLRLELDTMKHQSQL

SEQ ID NO: 11 - DNA, 800 bp, Homo sapien
atkagcttccgcttctgacaacactagagatccctcccctccctcagggtatggccctccacttcatttttggtacataacatctttataggacaggggtaaaatcccaatactaacaggagaatgcttaggactctaacaggtttttgagaatgtgttggtaagggccactcaatccaatttttcttggtcctccttgtggtctaggaggacaggcaagggtgcagattttcaagaatgcatcagtaagggccactaaatccgaccttcctcgttcctccttgtggtctgggaggaaaactagtgtttctgttgctgtgtcagtgagcacaactattccgatcagcagggtccagggaccactgcaggttcttgggcagggggagaaacaaaacaaaccaaaaccatgggcrgttttgtctttcagatgggaaacactcaggcatcaacaggctcacctttgaaatgcatcctaagccaatgggacaaatttgacccacaaaccctggaaaaagaggtggctcattttttttgcactatggcttggccccaacattctctctctgatggggaaaaatggccacctgagggaagtacagattacaatactatcctgcagcttgaccttttctgtaagagggaaggcaaatggagtgaaataccttatgtccaagctttcttttcattgaaggagaatacactatgcaaagcttgaaatttacatcccacaggaggacctctcagcttacccccatatcctagcctccctatagctccccttcctattagtgataagcctc

SEQ ID NO: 12 - protein, 102 aa, Homo sapien
  VARIANT (1)...(102): Xaa = Any Amino Acid
MGXFVFQMGNTQASTGSPLKCILSQWDKFDPQTLEKEVAHFFCTMAWPQHSLSDGEKWPPEGSTDYNTILQLDLFCKREGKWSEIPYVQAFFSLKENTLCKA

SEQ ID NO: 13 - DNA, 1206 bp, Homo sapien
ggcacgaggaagttttgtgtactgaaaaagaaactgtcagaagcaaaagaaataaaatcacagttagagaaccaaaaagttaaatgggaacaagagctctgcagtgtgaggtttctcacactcatgaaaatgaaaattatctcttacatgaaaattgcatgttgaaaaaggaaattgccatgctaaaactggaaatagccacactgaaacaccaataccaggaaaaggaaaataaatactttgaggacattaagattttaaaagaaaagaatgctgaacttcagatgaccctaaaactgaaagaggaatcattaactaaaagggcatctcaatatagtgggcagcttaaagttctgatagctgagaacacaatgctcacttctaaattgaaggaaaaacaagacaaagaaatactagaggcagaaattgaatcacaccatcctagactggcttctgctgtacaagaccatgatcaaattgtgacatcaagaaaaagtcaagaacctgctttccacattgcaggagatgcttgtttgcaaagaaaaatgaatgttgatgtgagtagtacgatatataacaatgaggtgctccatcaaccactttctgaagctcaaaggaaatccaaaagcctaaaaattaatctcaattatgccggagatgctctaagagaaaatacattggtttcagaacatgcacaaagagaccaacgtgaaacacagtgtcaaatgaaggaagctgaacacatgtatcaaaacgaacaagataatgtgaacaaacacactgaacagcaggagtctctagatcagaaattatttcaactacaaagcaaaaatatgtggcttcaacagcaattagttcatgcacataagaaagctgacaacaaaagcaagataacaattgatattcattttcttgagaggaaaatgcaacatcatctcctaaaagagaaaaatgaggagatatttaattacaataaccatttaaaaaaccgtatatatcaatatgaaaaagagaaagcagaaacagaagttatataatagtataacactgccaaggagcggattatctcatcttcatcctgtaattccagtgtttgtcacgtggttgttgaataaatgaataaagaatgagaaaaccagaagctctgatacataatcataatgataattatttcaatgcacaactacgggtggtgctgctcgtgcc

SEQ ID NO: 14 - protein, 317 aa, Homo sapien
MGTRALQCEVSHTHENENYLLHENCMLKKEIAMLKLEIATLKHQYQEKENKYFEDIKILKEKNAELQMTLKLKEESLTKRASQYSGQLKVLIAENTMLTSKLKEKQDKEILEAEIESHHPRLASAVQDHDQIVTSRKSQEPAFHIAGDACLQRKMNVDVSSTIYNNEVLHQPLSEAQRKSKSLKINLNYAGDALRENTLVSEHAQRDQRETQCQMKEAEHMYQNEQDNVNKHTEQQESLDQKLFQLQSKNMWLQQQLVHAHKKADNKSKITIDIHFLERKMQHHLLKEKNEEIFNYNNHLKNRIYQYEKEKAETEVI

SEQ ID NO: 15 - DNA, 1665 bp, Homo sapien
gcaaactttcaagcagagcctcccgagaagccatctgccttcgagcctgccattgaaatgcaaaagtctgttccaaataaagccttggaattgaagaatgaacaaacattgagagcagatcagatgttcccttcagaatcaaaacaaaagaaggttgaagaaaattcttgggattctgagagtctccgtgagactgtttcacagaaggatgtgtgtgtacccaaggctacacatcaaaaagaaatggataaaataagtggaaaattagaagattcaactagcctatcaaaaatcttggatacagttcattcttgtgaaagagcaagggaacttcaaaaagatcactgtgaacaacgtacaggaaaaatggaacaaatgaaaaagaagttttgtgtactgaaaaagaaactgtcagaagcaaaagaaataaaatcacagttagagaaccaaaaagttaaatgggaacaagagctctgcagtgtgaggtttctcacactcatgaaaatgaaaattatctcttacatgaaaattgcatgttgaaaaaggaaattgccatgctaaaactggaaatagccacactgaaacaccaataccaggaaaaggaaaataaatactttgaggacattaagattttaaaagaaaagaatgctgaacttcagatgaccctaaaactgaaagaggaatcattaactaaaagggcatctcaatatagtgggcagcttaaagttctgatagctgagaacacaatgctcacttctaaattgaaggaaaaacaagacaaagaaatactagaggcagaaattgaatcacaccatcctagactggcttctgctgtacaagaccatgatcaaattgtgacatcaagaaaaagtcaagaacctgctttccacattgcaggagatgcttgtttgcaaagaaaaatgaatgttgatgtgagtagtacgatatataacaatgaggtgctccatcaaccactttctgaagctcaaaggaaatccaaaagcctaaaaattaatctcaattatgccggagatgctctaagagaaaatacattggtttcagaacatgcacaaagagaccaacgtgaaacacagtgtcaaatgaaggaagctgaacacatgtatcaaaacgaacaagataatgtgaacaaacacactgaacagcaggagtctctagatcagaaattatttcaactacaaagcaaaaatatgtggcttcaacagcaattagttcatgcacataagaaagctgacaacaaaagcaagataacaattgatattcattttcttgagaggaaaatgcaacatcatctcctaaaagagaaaaatgaggagatatttaattacaataaccatttaaaaaaccgtatatatcaatatgaaaaagagaaagcagaaacagaaaactcatgagagacaagcagtaagaaacttcttttggagaaacaacagaccagatctttactcacaactcatgctaggaggccagtcctagcattaccttatgttgaaaatcttaccaatagtctgtgtcaacagaatacttattttagaagaaaaattcatgatttcttcctgaagcctgggcgacagagcgagactctgtctcaaaaaaaaaaaaaaaaaaagaaagaaagaaatgcctgtgcttacttcgcttcccagg

SEQ ID NO: 16 - protein, 179 aa, Homo sapien
ANFQAEPPEKPSAFEPAIEMQKSVPNKALELKNEQTLRADQMFPSESKQKKVEENSWDSESLRETVSQKDVCVPKATHQKEMDKISGKLEDSTSLSKILDTVHSCERARELQKDHCEQRTGKMEQMKKKFCVLKKKLSEAKEIKSQLENQKVKWEQELCSVRFLTLMKMKIISYMKIAC

SEQ ID NO: 17 - DNA, 1681 bp, Homo sapien
gatacagtcattcttgtgaaagagcaagggaacttcaaaaagatcactgtgaacaacgtacaggaaaaatggaacaaatgaaaaagaagttttgtgtactgaaaaagaaactgtcagaagcaaaagaaataaaatcacagttagagaaccaaaaagttaaatgggaacaagagctctgcagtgtgagattgactttaaaccaagaagaagagaagagaagaaatgccgatatattaaatgaaaaaattagggaagaattaggaagaatcgaagagcagcataggaaagagttagaagtgaaacaacaacttgaacaggctctcagaatacaagatatagaattgaagagtgtagaaagtaatttgaatcaggtttctcacactcatgaaaatgaaaattatctcttacatgaaaattgcatgttgaaaaaggaaattgccatgctaaaactggaaatagccacactgaaacaccaataccaggaaaaggaaaataaatactttgaggacattaagattttaaaagaaaagaatgctgaacttcagatgaccctaaaactgaaagaggaatcattaactaaaagggcatctcaatatagtgggcagcttaaagttctgatagctgagaacacaatgctcacttctaaattgaaggaaaaacaagacaaagaaatactagaggcagaaattgaatcacaccatcctagactggcttctgctgtacaagaccatgatcaaattgtgacatcaagaaaaagtcaagaacctgctttccacattgcaggagatgcttgtttgcaaagaaaaatgaatgttgatgtgagtagtacgatatataacaatgaggtgctccatcaaccactttctgaagctcaaaggaaatccaaaagcctaaaaattaatctcaattatgccggagatgctctaagagaaaatacattggtttcagaacatgcacaaagagaccaacgtgaaacacagtgtcaaatgaaggaagctgaacacatgtatcaaaacgaacaagataatgtgaacaaacacactgaacagcaggagtctctagatcagaaattatttcaactacaaagcaaaaatatgtggcttcaacagcaattagttcatgcacataagaaagctgacaacaaaagcaagataacaattgatattcattttcttgagaggaaaatgcaacatcatctcctaaaagagaaaaatgaggagatatttaattacaataaccatttaaaaaaccgtatatatcaatatgaaaaagagaaagcagaaacagaaaactcatgagagacaagcagtaagaaacttcttttggagaaacaacagaccagatctttactcacaactcatgctaggaggccagtcctagcattaccttatgttgaaaaatcttaccaatagtctgtgtcaacagaatacttattttagaagaaaaattcatgatttcttcctgaagcctacagacataaaataacagtgtgaagaattacttgttcacgaattgcataaaagctgcccaggatttccatctaccctggatgatgccggagacatcattcaatccaaccagaatctcgctctgtcactcaggctggagtgcagtgggcgcaatctcggctcactgcaactctgcctcccaggttcacgccattctctggcacagcctcccg

SEQ ID NO: 18 - protein, 432 aa, Homo sapien
DTVHSCERARELQKDHCEQRTGKMEQMKKKFCVLKKKLSEAKEIKSQLENQKVKWEQELCSVRLTLNQEEEKRRNADILNEKIREELGRIEEQHRKELEVKQQLEQALRIQDIELKSVESNLNQVSHTHENENYLLHENCMLKKEIAMLKLEIATLKHQYQEKENKYFEDIKILKEKNAELQMTLKLKEESLTKRASQYSGQLKVLIAENTMLTSKLKEKQDKEILEAEIESHHPRLASAVQDHDQIVTSRKSQEPAFHIAGDACLQRKMNVDVSSTIYNNEVLHQPLSEAQRKSKSLKINLNYAGDALRENTLVSEHAQRDQRETQCQMKEAEHMYQNEQDNVNKHTEQQESLDQKLFQLQSKNMWLQQQLVHAHKKADNKSKITIDIHFLERKMQHHLLKEKNEEIFNYNNHLKNRIYQYEKEKAETENS

SEQ ID NO: 19 - DNA, 3681 bp, Homo sapiens
tccgagctgattacagacaccaaggaagatgctgtaaagagtcagcagccacagccctggctagctggccctgtgggcatttattagtaaagttttaatgacaaaagctttgagtcaacacacccgtgggtaattaacctggtcatccccaccctggagagccatcctgcccatgggtgatcaaagaaggaacatctgcaggaacacctgatgaggctgcacccttggcggaaagaacacctgacacagctgaaagcttggtggaaaaaacacctgatgaggctgcacccttggtggaaagaacacctgacacggctgaaagcttggtggaaaaaacacctgatgaggctgcatccttggtggagggaacatctgacaaaattcaatgtttggagaaagcgacatctggaaagttcgaacagtcagcagaagaaacacctagggaaattacgagtcctgcaaaagaaacatctgagaaatttacgtggccagcaaaaggaagacctaggaagatcgcatgggagaaaaaagaagacacacctagggaaattatgagtcccgcaaaagaaacatctgagaaatttacgtgggcagcaaaaggaagacctaggaagatcgcatgggagaaaaaagaaacacctgtaaagactggatgcgtggcaagagtaacatctaataaaactaaagttttggaaaaaggaagatctaagatgattgcatgtcctacaaaagaatcatctacaaaagcaagtgccaatgatcagaggttcccatcagaatccaaacaagaggaagatgaagaatattcttgtgattctcggagtctctttgagagttctgcaaagattcaagtgtgtatacctgagtctatatatcaaaaagtaatggagataaatagagaagtagaagagcctcctaagaagccatctgccttcaagcctgccattgaaatgcaaaactctgttccaaataaagcctttgaattgaagaatgaacaaacattgagagcagatccgatgttcccaccagaatccaaacaaaaggactatgaagaaaattcttgggattctgagagtctctgtgagactgtttcacagaaggatgtgtgtttacccaaggctacacatcaaaaagaaatagataaaataaatggaaaattagaagagtctcctaataaagatggtcttctgaaggctacctgcggaatgaaagtttctattccaactaaagccttagaattgaaggacatgcaaactttcaaagcagagcctccggggaagccatctgccttcgagcctgccactgaaatgcaaaagtctgtcccaaataaagccttggaattgaaaaatgaacaaacattgagagcagatgagatactcccatcagaatccaaacaaaaggactatgaagaaagttcttgggattctgagagtctctgtgagactgtttcacagaaggatgtgtgtttacccaaggctrcrcatcaaaaagaaatagataaaataaatggaaaattagaagggtctcctgttaaagatggtcttctgaaggctaactgcggaatgaaagtttctattccaactaaagccttagaattgatggacatgcaaactttcaaagcagagcctcccgagaagccatctgccttcgagcctgccattgaaatgcaaaagtctgttccaaataaagccttggaattgaagaatgaacaaacattgagagcagatgagatactcccatcagaatccaaacaaaaggactatgaagaaagttcttgggattctgagagtctctgtgagactgtttcacagaaggatgtgtgtttacccaaggctrcrcatcaaaaagaaatagataaaataaatggaaaattagaagagtctcctgataatgatggttttctgaaggctccctgcagaatgaaagtttctattccaactaaagccttagaattgatggacatgcaaactttcaaagcagagcctcccgagaagccatctgccttcgagcctgccattgaaatgcaaaagtctgttccaaataaagccttggaattgaagaatgaacaaacattgagagcagatcagatgttcccttcagaatcaaaacaaaagaasgttgaagaaaattcttgggattctgagagtctccgtgagactgtttcacagaaggatgtgtgtgtacccaaggctacacatcaaaaagaaatggataaaataagtggaaaattagaagattcaactagcctatcaaaaatcttggatacagttcattcttgtgaaagagcaagggaacttcaaaaagatcactgtgaacaacgtacaggaaaaatggaacaaatgaaaaagaagttttgtgtactgaaaaagaaactgtcagaagcaaaagaaataaaatcacagttagagaaccaaaaagttaaatgggaacaagagctctgcagtgtgaggtttctcacactcatgaaaatgaaaattatctcttacatgaaaattgcatgttgaaaaaggaaattgccatgctaaaactggaaatagccacactgaaacaccaataccaggaaaaggaaaataaatactttgaggacattaagattttaaaagaaaagaatgctgaacttcagatgaccctaaaactgaaagaggaatcattaactaaaagggcatctcaatatagtgggcagcttaaagttctgatagctgagaacacaatgctcacttctaaattgaaggaaaaacaagacaaagaaatactagaggcagaaattgaatcacaccatcctagactggcttctgctgtacaagaccatgatcaaattgtgacatcaagaaaaagtcaagaacctgctttccacattgcaggagatgcttgtttgcaaagaaaaatgaatgttgatgtgagtagtacgatatataacaatgaggtgctccatcaaccactttctgaagctcaaaggaaatccaaaagcctaaaaattaatctcaattatgcmggagatgctctaagagaaaatacattggtttcagaacatgcacaaagagaccaacgtgaaacacagtgtcaaatgaaggaagctgaacacatgtatcaaaacgaacaagataatgtgaacaaacacactgaacagcaggagtctctagatcagaaattatttcaactacaaagcaaaaatatgtggcttcaacagcaattagttcatgcacataagaaagctgacaacaaaagcaagataacaattgatattcattttcttgagaggaaaatgcaacatcatctcctaaaagagaaaaatgaggagatatttaattacaataaccatttaaaaaaccgtatatatcaatatgaaaaagagaaagcagaaacagaaaactcatgagagacaagcagtaagaaacttcttttggagaaacaacagaccagatctttactcacaactcatgctaggaggccagtcctagcatcaccttatgttgaaaatcttaccaatagtctgtgtcaacagaatacttattttagaagaaaaattcatgatttcttcctgaagcctacagacataaaataacagtgtgaagaattacttgttcacgaattgcataaagctgcacaggattcccatctaccctgatgatgcagcagacatcattcaatccaaccagaatctcgctctgtcactcaggctgg

SEQ ID NO: 20 - DNA, 1424 bp, Homo sapiens
tccgagctgattacagacaccaaggaagatgctgtaaagagtcagcagccacagccctggctagctggccctgtgggcatttattagtaaagttttaatgacaaaagctttgagtcaacacacccgtgggtaattaacctggtcatccccaccctggagagccatcctgcccatgggtgatcaaagaaggaacatctgcaggaacacctgatgaggctgcacccttggcggaaagaacacctgacacagctgaaagcttggtggaaaaaacacctgatgaggctgcacccttggtggaaagaacacctgacacggctgaaagcttggtggaaaaaacacctgatgaggctgcatccttggtggagggaacatctgacaaaattcaatgtttggagaaagcgacatctggaaagttcgaacagtcagcagaagaaacacctagggaaattacgagtcctgcaaaagaaacatctgagaaatttacgtggccagcaaaaggaagacctaggaagatcgcatgggagaaaaaagaagacacacctagggaaattatgagtcccgcaaaagaaacatctgagaaatttacgtgggcagcaaaaggaagacctaggaagatcgcatgggagaaaaaagaaacacctgtaaagactggatgcgtggcaagagtaacatctaataaaactaaagttttggaaaaaggaagatctaagatgattgcatgtcctacaaaagaatcatctacaaaagcaagtgccaatgatcagaggttcccatcagaatccaaacaagaggaagatgaagaatattcttgtgattctcggagtctctttgagagttctgcaaagattcaagtgtgtatacctgagtctatatatcaaaaagtaatggagataaatagagaagtagaagagcctcctaagaagccatctgccttcaagcctgccattgaaatgcaaaactctgttccaaataaagcctttgaattgaagaatgaacaaacattgagagcagatccgatgttcccaccagaatccaaacaaaaggactatgaagaaaattcttgggattctgagagtctctgtgagactgtttcacagaaggatgtgtgtttacccaaggctacacatcaaaaagaaatagataaaataaatggaaaattagaaggtaagaaccgttttttatttaaaaatcagttgaccgaatatttctctaaactgatgaggagggatatcctctagtagctgaagaaaattacctcctaaatgcaaaccatggaaaaaaagagaagtgcaatggtcgtaagttgtatgtctcatcaggtgttggcaacagactatattgagagtgctgaaaaggagctgaattattagtttgaattcaagatattgcaagacctgagagaaaaaaaaaaaaaaaaaaaaaaa

SEQ ID NO: 21 - DNA, 674 bp, Homo sapiens
attccgagctgattacagacaccaaggaagatgctgtaaagagtcagcagccacagccctggctagctggccctgtgggcatttattagtaaagttttaatgacaaaagctttgagtcaacacacccgtgggtaattaacctggtcatccccaccctggagagccatcctgcccatgggtgatcaaagaaggaacatctgcaggaacacctgatgaggctgcacccttggcggaaagaacacctgacacagctgaaagcttggtggaaaaaacacctgatgaggctgcacccttggtggaaagaacacctgacacggctgaaagcttggtggaaaaaacacctgatgaggctgcatccttggtggagggaacatctgacaaaattcaatgtttggagaaagcgacatctggaaagttcgaacagtcagcagaagaaacacctagggaaattacgagtcctgcaaaagaaacatctgagaaatttacgtggccagcaaaaggaagacctaggaagatcgcatgggagaaaaaagatgactcagttaaggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 22 - DNA, 1729 bp, Homo sapiens
  unsure (11): n=A,T,C or G
gaaagttcgancagtcagcagaagaaacacctagggaaattacgagtcctgcaaaagaaacatctgagaaatttacgtggccagcaaaaggaagacctaggaagatcgcatgggagaaaaaagaagacacacctagggaaattatgagtcccgcaaaagaaacatctgagaaatttacgtgggcagcaaaaggaagacctaggaagatcgcatgggagaaaaaagaaacacctgtaaagactggatgcgtggcaagagtaacatctaataaaactaaagttttggaaaaaggaagatctaagatgattgcatgtcctacaaaagaatcatctacaaaagcaagtgccaatgatcagaggttcccatcagaatccaaacaagaggaagatgaagaatattcttgtgattctcggagtctctttgagagttctgcaaagattcaagtgtgtatacctgagtctatatatcaaaaagtaatggagataaatagagaagtagaagagcctcctaagaagccatctgccttcaagcctgccattgaaatgcaaaactctgttccaaataaagcctttgaattgaagaatgaacaaacattgagagcagatccgatgttcccaccagaatccaaacaaaaggactatgaagaaaattcttgggattctgagagtctctgtgagactgtttcacagaaggatgtgtgtttacccaaggctacacatcaaaaagaaatagataaaataaatggaaaattagaagagtctcctaataaagatggtcttctgaaggctacctgcggaatgaaagtttctattccaactaaagccttagaattgaaggacatgcaaactttcaaagcagagcctccggggaagccatctgccttcgagcctgccactgaaatgcaaaagtctgtcccaaataaagccttggaattgaaaaatgaacaaacattgagagcagatgagatactcccatcagaatccaaacaaaaggactatgaagaaaattcttgggatactgagagtctctgtgagactgtttcacagaaggatgtgtgtttacccaaggctgcgcatcaaaaagaaatagataaaataaatggaaaattagaagggtctcctggtaaanatggtcttctgaaggctaactgcggaatgaaagtttctattccaactaaagccttagaattgatggacatgcaaactttcaaagcagagcctcccgagaagccatctgccttcgagcctgccattgaaatgcaaaagtctgttccaaataaagccttggaattgaagaatgaacaaacattgagagcagatgagatactcccatcagaatccaaacaaaaggactatgaagaaagttcttgggattctgagagtctctgtgagactgtttcacagaaggatgtgtgtttacccaaggctgcgcatcaaaaagaaatagataaaataaatggaaaattagaaggtaagaaccgttttttatttaaaaatcatttgaccaaatatttctctaaattgatgaggaaggatatcctctagtagctgaagaaaattacctcctaaatgcaaaccatggaaaaaaagagaagtgcaatggtcataagctatgtgtctcatcaggcattggcaacagactatattgtgagtgctgaagaggagctgaattactagtttaaattcaagatattccaagacgtgaggaaaatgagaaaaaaaaaaaaaaa

SEQ ID NO: 23 - DNA, 1337 bp, Homo sapiens
aaaaagaaatagataaaataaatggaaaattagaagggtctcctgttaaagatggtcttctgaaggctaactgcggaatgaaagtttctattccaactaaagccttagaattgatggacatgcaaactttcaaagcagagcctcccgagaagccatctgccttcgagcctgccattgaaatgcaaaagtctgttccaaataaagccttggaattgaagaatgaacaaacattgagagcagatgagatactcccatcagaatccaaacaaaaggactatgaagaaagttcttgggattctgagagtctctgtgagactgtttcacagaaggatgtgtgtttacccaaggctgcgcatcaaaaagaaatagataaaataaatggaaaattagaagagtctcctgataatgatggttttctgaaggctccctgcagaatgaaagtttctattccaactaaagccttagaattgatggacatgcaaactttcaaagcagagcctcccgagaagccatctgccttcgagcctgccattgaaatgcaaaagtctgttccaaataaagccttggaattgaagaatgaacaaacattgagagcagatcagatgttcccttcagaatcaaaacaaaagaaggttgaagaaaattcttgggattctgagagtctccgtgagactgtttcacagaaggatgtgtgtgtacccaaggctacacatcaaaaagaaatggataaaataagtggaaaattagaagattcaactagcctatcaaaaatcttggatacagttcattcttgtgaaagagcaagggaacttcaaaaagatcactgtgaacaacgtacaggaaaaatggaacaaatgaaaaagaagttttgtgtactgaaaaagaaactgtcagaagcaaaagaaataaaatcacagttagagaaccaaaaagttaaatgggaacaagagctctgcagtgtgagattgactttaaaccaagaagaagagaagagaagaaatgccgatatattaaatgaaaaaattagggaagaattaggaagaatcgaagagcagcataggaaagagttagaagtgaaacaacaacttgaacaggctctcagaatacaagatatagaattgaagagtgtagaaagtaatttgaatcaggtttctcacactcatgaaaatgaaaattatctcttacatgaaaattgcatgttgaaaaaggaaattgccatgctaaaactggaaatagccacactgaaacaccaataccaggaaaaggaaaataaatactttgaggacattaagattttaaaagaaaagaatgctgaacttcagatgacccctcgtgcc

SEQ ID NO: 24 - DNA, 2307 bp, Homo sapiens
attgagagcagatgagatactcccatcagaatccaaacaaaaggactatgaagaaagttcttgggattctgagagtctctgtgagactgtttcacagaaggatgtgtgtttacccaaggctacacatcaaaaagaaatagataaaataaatggaaaattagaagggtctcctgttaaagatggtcttctgaaggctaactgcggaatgaaagtttctattccaactaaagccttagaattgatggacatgcaaactttcaaagcagagcctcccgagaagccatctgccttcgagcctgccattgaaatgcaaaagtctgttccaaataaagccttggaattgaagaatgaacaaacattgagagcagatgagatactcccatcagaatccaaacaaaaggactatgaagaaagttcttgggattctgagagtctctgtgagactgtttcacagaaggatgtgtgtttacccaaggctacacatcaaaaagaaatagataaaataaatggaaaattagaagagtctcctgataatgatggttttctgaagtctccctgcagaatgaaagtttctattccaactaaagccttagaattgatggacatgcaaactttcaaagcagagcctcccgagaagccatctgccttcgagcctgccattgaaatgcaaaagtctgttccaaataaagccttggaattgaagaatgaacaaacattgagagcagatcagatgttcccttcagaatcaaaacaaaagaacgttgaagaaaattcttgggattctgagagtctccgtgagactgtttcacagaaggatgtgtgtgtacccaaggctacacatcaaaaagaaatggataaaataagtggaaaattagaagattcaactagcctatcaaaaatcttggatacagttcattcttgtgaaagagcaagggaacttcaaaaagatcactgtgaacaacgtacaggaaaaatggaacaaatgaaaaagaagttttgtgtactgaaaaagaaactgtcagaagcaaaagaaataaaatcacagttagagaaccaaaaagttaaatgggaacaagagctctgcagtgtgaggtttctcacactcatgaaaatgaaaattatctcttacatgaaaattgcatgttgaaaaaggaaattgccatgctaaaactggaaatagccacactgaaacaccaataccaggaaaaggaaaataaatactttgaggacattaagattttaaaagaaaagaatgctgaacttcagatgaccctaaaactgaaagaggaatcattaactaaaagggcatctcaatatagtgggcagcttaaagttctgatagctgagaacacaatgctcacttctaaattgaaggaaaaacaagacaaagaaatactagaggcagaaattgaatcacaccatcctagactggcttctgctgtacaagaccatgatcaaattgtgacatcaagaaaaagtcaagaacctgctttccacattgcaggagatgcttgtttgcaaagaaaaatgaatgttgatgtgagtagtacgatatataacaatgaggtgctccatcaaccactttctgaagctcaaaggaaatccaaaagcctaaaaattaatctcaattatgcaggagatgctctaagagaaaatacattggtttcagaacatgcacaaagagaccaacgtgaaacacagtgtcaaatgaaggaagctgaacacatgtatcaaaacgaacaagataatgtgaacaaacacactgaacagcaggagtctctagatcagaaattatttcaactacaaagcaaaaatatgtggcttcaacagcaattagttcatgcacataagaaagctgacaacaaaagcaagataacaattgatattcattttcttgagaggaaaatgcaacatcatctcctaaaagagaaaaatgaggagatatttaattacaataaccatttaaaaaaccgtatatatcaatatgaaaaagagaaagcagaaacagaaaactcatgagagacaagcagtaagaaacttcttttggagaaacaacagaccagatctttactcacaactcatgctaggaggccagtcctagcatcaccttatgttgaaaatcttaccaatagtctgtgtcaacagaatacttattttagaagaaaaattcatgatttcttcctgaagcctacagacataaaataacagtgtgaagaattacttgttcacgaattgcataaagctgcacaggattcccatctaccctgatgatgcagcagacatcattcaatccaaccagaatctcgc

SEQ ID NO: 25 - protein, 650 aa, Homo sapiens
  unsure (310): Xaa = Any Amino Acid
MSPAKETSEKFTWAAKGRPRKIAWEKKETPVKTGCVARVTSNKTKVLEKGRSKMIACPTKESSTKASANDQRFPSESKQEEDEEYSCDSRSLFESSAKIQVCIPESIYQKVMEINREVEEPPKKPSAFKPAIEMQNSVPNKAFELKNEQTLRADPMFPPESKQKDYEENSWDSESLCETVSQKDVCLPKATHQKEIDKINGKLEESPNKDGLLKATCGMKVSIPTKALELKDMQTFKAEPPGKPSAFEPATEMQKSVPNKALELKNEQTLRADEILPSESKQKDYEESSWDSESLCETVSQKDVCLPKAXHQKEIDKINGKLEGSPVKDGLLKANCGMKVSIPTKALELMDMQTFKAEPPEKPSAFEPAIEMQKSVPNKALELKNEQTLRADEILPSESKQKDYEESSWDSESLCETVSQKDVCLPKAXHQKEIDKINGKLEESPDNDGFLKAPCRMKVSIPTKALELMDMQTFKAEPPEKPSAFEPAIEMQKSVPNKALELKNEQTLRADQMFPSESKQKXVEENSWDSESLRETVSQKDVCVPKATHQKEMDKISGKLEDSTSLSKILDTVHSCERARELQKDHCEQRTGKMEQMKKKFCVLKKKLSEAKEIKSQLENQKVKWEQELCSVRFLTLMKMKIISYMKIAC

SEQ ID NO: 26 - protein, 228 aa, Homo sapiens
MSPAKETSEKFTWAAKGRPRKIAWEKKETPVKTGCVARVTSNKTKVLEKGRSKMIACPTKESSTKASANDQRFPSESKQEEDEEYSCDSRSLFESSAKIQVCIPESIYQKVMEINREVEEPPKKPSAFKPAIEMQNSVPNKAFELKNEQTLRADPMFPPESKQKDYEENSWDSESLCETVSQKDVCLPKATHQKEIDKINGKLEGKNRFLFKNQLTEYFSKLMRRDIL

SEQ ID NO: 27 - protein, 154 aa, Homo sapiens
  unsure (148): Xaa = Any Amino Acid
MRLHPWRKEHLTQLKAWWKKHLMRLHPWWKEHLTRLKAWWKKHLMRLHPWWREHLTKFNVWRKRHLESSNSQQKKHLGKLRVLQKKHLRNLRGQQKEDLGRSHGRKKMTQLRQKKKKKKKKKKKKKKKKKKKKKKKKKKKKKKKKKKXKKKKKK

SEQ ID NO: 28 - protein, 466 aa, Homo sapiens
  unsure (329): Xaa = Any Amino Acid
MSPAKETSEKFTWAAKGRPRKIAWEKKETPVKTGCVARVTSNKTKVLEKGRSKMIACPTKESSTKASANDQRFPSESKQEEDEEYSCDSRSLFESSAKIQVCIPESIYQKVMEINREVEEPPKKPSAFKPAIEMQNSVPNKAFELKNEQTLRADPMFPPESKQKDYEENSWDSESLCETVSQKDVCLPKATHQKEIDKINGKLEESPNKDGLLKATCGMKVSIPTKALELKDMQTFKAEPPGKPSAFEPATEMQKSVPNKALELKNEQTLRADEILPSESKQKDYEENSWDTESLCETVSQKDVCLPKAAHQKEIDKINGKLEGSPGKXGLLKANCGMKVSIPTKALELMDMQTFKAEPPEKPSAFEPAIEMQKSVPNKALELKNEQTLRADEILPSESKQKDYEESSWDSESLCETVSQKDVCLPKAAHQKEIDKINGKLEGKNRFLFKNHLTKYFSKLMRKDIL

SEQ ID NO: 29 - protein, 445 aa, Homo sapiens
KEIDKINGKLEGSPVKDGLLKANCGMKVSIPTKALELMDMQTFKAEPPEKPSAFEPAIEMQKSVPNKALELKNEQTLRADEILPSESKQKDYEESSWDSESLCETVSQKDVCLPKAAHQKEIDKINGKLEESPDNDGFLKAPCRMKVSIPTKALELMDMQTFKAEPPEKPSAFEPAIEMQKSVPNKALELKNEQTLRADQMFPSESKQKKVEENSWDSESLRETVSQKDVCVPKATHQKEMDKISGKLEDSTSLSKILDTVHSCERARELQKDHCEQRTGKMEQMKKKFCVLKKKLSEAKEIKSQLENQKVKWEQELCSVRLTLNQEEEKRRNADILNEKIREELGRIEEQHRKELEVKQQLEQALRIQDIELKSVESNLNQVSHTHENENYLLHENCMLKKEIAMLKLEIATLKHQYQEKENKYFEDIKILKEKNAELQMTPRA

SEQ ID NO: 30 - DNA, 578 bp, Human
cttgccttctcttaggctttgaagcatttttgtctgtgctccctgatcttcaggtcaccaccatgaagttcttagcagtcctggtactcttgggagtttccatctttctggtctctgcccagaatccgacaacagctgctccagctgacacgtatccagctactggtcctgctgatgatgaagcccctgatgctgaaaccactgctgctgcaaccactgcgaccactgctgctcctaccactgcaaccaccgctgcttctaccactgctcgtaaagacattccagttttacccaaatgggttggggatctcccgaatggtagagtgtgtccctgagatggaatcagcttgagtcttctgcaattggtcacaactattcatgcttcctgtgatttcatccaactacttaccttgcctacgatatcccctttatctctaatcagtttattttctttcaaataaaaaataactatgagcaacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - protein, 90 aa, Homo sapien
MKFLAVLVLLGVSIFLVSAQNPTTAAPADTYPATGPADDEAPDAETTAAATTATTAAPTTATTAASTTARKDIPVLPKWVGDLPNGRVCP

SEQ ID NO: 32 - DNA, 3101 bp, Homo sapien
tgttggggcctcagcctcccaagtagctgggactacaggtgcctgccaccacgcccagctaattttttgtatattttttagtagagacggggtttcaccgtggtctcaatctcctgacctcgtgatctgccagccttggcctcccaaagtgtattctctttttattattattattatttttgagatggagtctgtctctgtcgcccaggctggagtgcagtggtgcgatctctgctcactgcaagctccgcctcctgggttcatgccattctcctgcctcagcctcccgagtagctgggactacaggcccctgccaccacacccggctaattttttgtatttttagtagagacagggtttcaccatgttagccagggtggtctctatcttctgacctcgtgatccgcctgcctcagtctctcaaagtgctgggattacaggcgtgagccaccgcgaccagccaactattgctgtttatttttaaatatattttaaagaaacaattagatttgttttctttctcattcttttacttctactcttcatgtatgtataattatatttgtgttttctattaccttttctccttttactgtattggactataataattgtgctcactaatttctgttcactaatattatcagcttagataatactttaatttttaacttatatattgagtattaaattgatcagttttatttgtaattatctatcttccgcttggctgaatataacttcttaagcttataacttcttgttctttccatgttatttttttcttttttttaatgtattgaatttcttctgacactcattctagtaacttttttctcggtgtgcaacgtaagttataatttgtttctcagatttgagatctgccataagtttgaggctttattttttttttttatttgctttatggcaagtcggacaacctgcatggatttggcatcaatgtagtcacccatatctaagagcagcacttgcttcttagcatgatgagttgtttctggattgtttctttattttacttatattcctggtagattcttatattttcccttcaactctattcagcattttaggaattcttaggactttctgagaattttagctttctgtattaaatgtttttaatgagtattgcattttctcaaaaagcacaaatatcaatagtgtacacatgaggaaaactatatatatattctgttgcagatgacagcatctcataacaaaatcctagttacttcatttaaaagacagctctcctccaatatactatgaggtaacaaaaatttgtagtgtgtaatttttttaatattagaaaactcatcttacattgtgcacaaatttctgaagtgataatacttcactgtttttctatagaagtaacttaatattggcaaaattacttatttgaatttaggttttggctttcatcatatacttcctcattaacatttccctcaatccataaatgcaatctcagtttgaatcttccatttaacccagaagttaatttttaaaaccttaataaaatttgaatgtagctagatattatttgttggttacatattagtcaataatttatattacttacaatgatcagaaaatatgatctgaatttctgctgtcataaattcaataacgtattttaggcctaaacctttccatttcaaatccttgggtctggtaattgaaaataatcattatcttttgttttctggccaaaaatgctgcccatttatttctatccctaattagtcaaactttctaataaatgtatttaacgttaatgatgtttatttgcttgttgtatactaaaaccattagtttctataatttaaatgtcacctaatatgagtgaaaatgtgtcagaggctggggaagaatgtggatggagaaagggaaggtgttgatcaaaaagtacccaagtttcagttacacaggaggcatgagattgatctagtgcaaaaaatgatgagtataataaataataatgcactgtatattttgaaattgctaaaagtagatttaaaattgatttacataatattttacatatttataaagcacatgcaatatgttgttacatgtatagaatgtgcaacgatcaagtcagggtatctgtggtatccaccactttgagcatttatcgattctatatgtcaggaacatttcaagttatctgttctagcaaggaaatataaaatacattatagttaactatggcctatctacagtgcaactaaacactagattttattcctttccaactgtgggtttgtattcatttaccaccctcttttcattccctttctcacccacacactgtgccgggcctcaggcatatactattctactgtctgtctctgtaaggattatcattttagcttccacatatgagagaatgcatgcaaagtttttctttccatgtctggcttatttcacttaacaaaatgacctccgcttccatccatgttatttatattacccaatagtgttcataaatatatatacacacatatataccacattgcatttgtccaattattcattgacggaaactggttaatgttatatcgttgctattgtgaatagtgctgcaataaacacgcaagtggggatataatttgaagagtttttttgttgatgttccatacaaattttaagattgttttgtctatgtttgtgaaaatggcgttagtattttcatagagattgcattgaatctgtagattgctttgggtaagtatggttattttgatggtattaattttttcattccatgaagatgagatgtctttccatttgtttgtgtcctctacattttctttcatcaaagttttgttgtatttttgaagtagatgtatttcaccttatagatcaagtgtattccctaaatattttatttttgtagctattgtagatgaaattgccttctcgatttctttttcacttaattcattattagtgtatggaaatgttatggatttttatttgttggtttttaatcaaaaactgtattaaacttagagttttttgtggagtttttaagtttttctagatataagatcatgacatctaccaaaaaa

SEQ ID NO: 33 - DNA, 16 bp, Artificial Sequence
  PCR primer
tgcccctccggaagct

SEQ ID NO: 34 - DNA, 23 bp, Artificial Sequence
  PCR primer
cgtttctgaagggacatctgatc

SEQ ID NO: 35 - DNA, 30 bp, Artificial Sequence
  PCR primer
ttgcagccaagttaggagtgaagagatgca

SEQ ID NO: 36 - DNA, 24 bp, Artificial Sequence
  PCR primer
aagcctcagagtccttccagtatg

SEQ ID NO: 37 - DNA, 35 bp, Artificial Sequence
  PCR primer
ttcaaatataagtgaagaaaaaattagtagatcaa

SEQ ID NO: 38 - DNA, 37 bp, Artificial Sequence
  PCR primer
aatccattgtatcttagaaccgagggatttgtttaga

SEQ ID NO: 39 - DNA, 22 bp, Artificial Sequence
  PCR primer
aaagcagatggtggttgaggtt

SEQ ID NO: 40 - DNA, 22 bp, Artificial Sequence
  PCR primer
cctgagaccaaatggcttcttc

SEQ ID NO: 41 - DNA, 24 bp, Artificial Sequence
  PCR primer
attccatgccggctgcttcttctg

SEQ ID NO: 42 - DNA, 30 bp, Artificial Sequence
  PCR primer
tctggttttctcattctttattcatttatt

SEQ ID NO: 43 - DNA, 20 bp, Artificial Sequence
  PCR primer
tgccaaggagcggattatct

SEQ ID NO: 44 - DNA, 30 bp, Artificial Sequence
  PCR primer
caaccacgtgacaaacactggaattacagg

SEQ ID NO: 45 - DNA, 21 bp, Artificial Sequence
  PCR primer
actggaacggtgaaggtgaca

SEQ ID NO: 46 - DNA, 20 bp, Artificial Sequence
  PCR primer
cggccacattgtgaactttg

SEQ ID NO: 47 - DNA, 23 bp, Artificial Sequence
  PCR primer
cagtcggttggagcgagcatccc

SEQ ID NO: 48 - DNA, 24 bp, Artificial Sequence
  PCR primer
tgccatagatgaattgaaggaatg

SEQ ID NO: 49 - DNA, 29 bp, Artificial Sequence
  PCR primer
tgtcatatattaattgcataaacacctca

SEQ ID NO: 50 - DNA, 32 bp, Artificial Sequence
  PCR primer
tcttaaccaaacggatgaaactctgagcaatg

SEQ ID NO: 51 - DNA, 28 bp, Artificial Sequence
  PCR primer
atcattgaaaattcaaatataagtgaag

SEQ ID NO: 52 - DNA, 30 bp, Artificial Sequence
  PCR primer
gtagttgtgcattgaaataattatcattat

SEQ ID NO: 53 - DNA, 20 bp, Artificial Sequence
  PCR Primer
caattttggtggagaacccg

SEQ ID NO: 54 - DNA, 20 bp, Artificial Sequence
  PCR Primer
gctgtcggaggtatatggtg

SEQ ID NO: 55 - DNA, 28 bp, Artificial Sequence
  PCR Primer
catttcagagagtaacatggactacaca

SEQ ID NO: 56 - DNA, 21 bp, Artificial Sequence
  PCR Primer
tctgataaaggccgtacaatg

SEQ ID NO: 57 - DNA, 22 bp, Artificial Sequence
  PCR Primer
tcacgacttgctgtttttgctc

SEQ ID NO: 58 - DNA, 30 bp, Artificial Sequence
  PCR Primer
atcaaaaaacaagcatggcctcacaccact

SEQ ID NO: 59 - DNA, 21 bp, Artificial Sequence
  PCR Primer
gcaagtgccaatgatcagagg

SEQ ID NO: 60 - DNA, 23 bp, Artificial Sequence
  PCR Primer
atatagactcaggtatacacact

SEQ ID NO: 61 - DNA, 30 bp, Artificial Sequence
  PCR Primer
tcccatcagaatccaaacaagaggaagatg

SEQ ID NO: 62 - DNA, 34 bp, Artificial Sequence
  PCR Primer
aatccattgtatcttagaaccgagggatttgttt

SEQ ID NO: 63 - DNA, 24 bp, Artificial Sequence
  PCR Primer
ccgcttctgacaacactagagatc

SEQ ID NO: 64 - DNA, 32 bp, Artificial Sequence
  PCR Primer
cctataaagatgttatgtaccaaaaatgaagt

SEQ ID NO: 65 - DNA, 22 bp, Artificial Sequence
  PCR Primer
cccctccctcagggtatggccc

SEQ ID NO: 66 - DNA, 22 bp, Artificial Sequence
  PCR Primer
ccctttctcacccacacactgt

SEQ ID NO: 67 - DNA, 24 bp, Artificial Sequence
  PCR Primer
tgcattctctcatatgtggaagct

SEQ ID NO: 68 - DNA, 33 bp, Artificial Sequence
  PCR Primer
ccgggcctcaggcatatactattctactgtctg

SEQ ID NO: 69 - DNA, 24 bp, Artificial Sequence
  PCR Primer
gacattccagttttacccaaatgg

SEQ ID NO: 70 - DNA, 23 bp, Artificial Sequence
  PCR Primer
tgcagaagactcaagctgattcc

SEQ ID NO: 71 - DNA, 28 bp, Artificial Sequence
  PCR Primer
tctcagggacacactctaccattcggga

SEQ ID NO: 72 - DNA, 30 bp, Artificial Sequence
  PCR Primer
aaatataagtgaagaaaaaaattagtagat

SEQ ID NO: 73 - DNA, 503 bp, Homo sapiens
gacagcggcttccttgatccttgccacccgcgactgaacaccgacagcagcagcctcaccatgaagttgctgatggtcctcatgctggcggccctctcccagcactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttctgcaattaatatatgacagcagtctttgtgatttattttaactttctgcaagacctttggctcacagaactgcagggtatggtgagaaaccaactacggattgctgcaaaccacaccttctctttcttatgtctttttactacaaactacaagacaattgttgaaacctgctatacatgtttattttaataaattgatggca

SEQ ID NO: 74 - DNA, 301 bp, Homo sapiens
cactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttatttggcggccatcaccatcaccatcactaaggtcccgagctcgaattctgcagatatccatcacact

SEQ ID NO: 75 - DNA, 3282 bp, Homo sapiens
gggacagggctgaggatgaggagaaccctggggacccagaagaccgtgccttgcccggaagtcctgcctgtaggcctgaaggacttgccctaacagagcctcaacaactacctggtgattcctacttcagccccttggtgtgagcagcttctcaacatgaactacagcctccacttggccttcgtgtgtctgagtctcttcactgagaggatgtgcatccaggggagtcagttcaacgtcgaggtcggcagaagtgacaagctttccctgcctggctttgagaacctcacagcaggatataacaaatttctcaggcccaattttggtggagaacccgtacagatagcgctgactctggacattgcaagtatctctagcatttcagagagtaacatggactacacagccaccatatacctccgacagcgctggatggaccagcggctggtgtttgaaggcaacaagagcttcactctggatgcccgcctcgtggagttcctctgggtgccagatacttacattgtggagtccaagaagtccttcctccatgaagtcactgtgggaaacaggctcatccgcctcttctccaatggcacggtcctgtatgccctcagaatcacgacaactgttgcatgtaacatggatctgtctaaataccccatggacacacagacatgcaagttgcagctggaaagctggggctatgatggaaatgatgtggagttcacctggctgagagggaacgactctgtgcgtggactggaacacctgcggcttgctcagtacaccatagagcggtatttcaccttagtcaccagatcgcagcaggagacaggaaattacactagattggtcttacagtttgagcttcggaggaatgttctgtatttcattttggaaacctacgttccttccactttcctggtggtgttgtcctgggtttcattttggatctctctcgattcagtccctgcaagaacctgcattggagtgacgaccgtgttatcaatgaccacactgatgatcgggtcccgcacttctcttcccaacaccaactgcttcatcaaggccatcgatgtgtacctggggatctgctttagctttgtgtttggggccttgctagaatatgcagttgctcactacagttccttacagcagatggcagccaaagatagggggacaacaaaggaagtagaagaagtcagtattactaatatcatcaacagctccatctccagctttaaacggaagatcagctttgccagcattgaaatttccagcgacaacgttgactacagtgacttgacaatgaaaaccagcgacaagttcaagtttgtcttccgagaaaagatgggcaggattgttgattatttcacaattcaaaaccccagtaatgttgatcactattccaaactactgtttcctttgatttttatgctagccaatgtattttactgggcatactacatgtatttttgagtcaatgttaaatttcttgcatgccataggtcttcaacaggacaagataatgatgtaaatggtattttaggccaagtgtgcacccacatccaatggtgctacaagtgactgaaataatatttgagtctttctgctcaaagaatgaagctccaaccattgttctaagctgtgtagaagtcctagcattataggatcttgtaatagaaacatcagtccattcctctttcatcttaatcaaggacattcccatggagcccaagattacaaatgtactcagggctgtttattcggtggctccctggtttgcatttacctcatataaagaatgggaaggagaccattgggtaaccctcaagtgtcagaagttgtttctaaagtaactatacatgttttttactaaatctctgcagtgcttataaaatacattgttgcctatttagggagtaacattttctagtttttgtttctggttaaaatgaaatatgggcttatgtcaattcattggaagtcaatgcactaactcaataccaagatgagtttttaaataatgaatattatttaataccacaacagaattatccccaatttccaataagtcctatcattgaaaattcaaatataagtgaagaaaaaattagtagatcaacaatctaaacaaatccctcggttctaagatacaatggattccccatactggaaggactctgaggctttattcccccactatgcatatcttatcattttattattatacacacatccatcctaaactatactaaagcccttttcccatgcatggatggaaatggaagatttttttgtaacttgttctagaagtcttaatatgggctgttgccatgaaggcttgcagaattgagtccattttctagctgcctttattcacatagtgatggggtactaaaagtactgggttgactcagagagtcgctgtcattctgtcattgctgctactctaacactgagcaacactctcccagtggcagatcccctgtatcattccaagaggagcattcatccctttgctctaatgatcaggaatgatgcttattagaaaacaaactgcttgacccaggaacaagtggcttagcttaagtaaacttggctttgctcagatccctgatccttccagctggtctgctctgagtggcttatcccgcatgagcaggagcgtgctggccctgagtactgaactttctgagtaacaatgagacacgttacagaacctatgttcaggttgcgggtgagctgccctctccaaatccagccagagatgcacattcctcggccagtctcagccaacagtaccaaaagtgatttttgagtgtgccagggtaaaggcttccagttcagcctcagttattttagacaatctcgccatctttaatttcttagcttcctgttctaataaatgcacggctttacctttcctgtcagaaataaaccaaggctctaaaagatgatttcccttctgtaactccctagagccacaggttctcattccttttcccattatacttctcacaattcagtttctatgagtttgatcacctgatttttttaacaaaatatttctaacgggaatgggtgggagtgctggtgaaaagagatgaaatgtggttgtatgagccaatcatatttgtgattttttaaaaaaagtttaaaaggaaatatctgttctgaaaccccacttaagcattgtttttatataaaaacaatgataaagatgtgaactgtgaaataaatataccatattagctacccaccaaaaaaaaaaaaaaaaaa

SEQ ID NO: 76 - DNA, 463 bp, Homo sapiens
tagaattcagcggccgcttaattctagaagtccaaatcactcattgtttgtgaaagctgagctcacagcaaaacaagccaccatgaagctgtcggtgtgtctcctgctggtcacgctggccctctgctgctaccaggccaatgccgagttctgcccagctcttgtttctgagctgttagacttcttcttcattagtgaacctctgttcaagttaagtcttgccaaatttgatgcccctccggaagctgttgcagccaagttaggagtgaagagatgcacggatcagatgtcccttcagaaacgaagcctcattgcggaagtcctggtgaaaatattgaagaaatgtagtgtgtgacatgtaaaaactttcatcctggtttccactgtctttcaatgacaccctgatcttcactgcagaatgtaaaggtttcaacgtcttgctttaataaatcacttgctctac

SEQ ID NO: 77 - protein, 90 aa, Homo sapiens
MKLSVCLLLVTLALCCYQANAEFCPALVSELLDFFFISEPLFKLSLAKFDAPPEAVAAKLGVKRCTDQMSLQKRSLIAEVLVKILKKCSV

(19) United States
(12) Patent Application Publication
(10) Pub. No.: US 2003/0170631 A1
(43) Pub. Date: Sep. 11, 2003

(54) Methods, compositions and kits for the detection and monitoring of breast cancer

(75) Inventors: Raymond L. Houghton, Bothell, WA (US); Davin C. Dillon, Issaquah, WA (US); David A. Molesh, Kingston, WA (US); Jiangchun Xu, Bellevue, WA (US); Barbara Zehentner, Bainbridge Island, WA (US); David H. Persing, Redmond, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Corixa Corporation, Seattle, WA (US)

(21) Appl. No.: 10/033,527

(22) Filed: Dec. 27, 2001

Related U.S. Application Data
(63) Continuation-in-part of application No. 09/825,301, filed on Apr. 2, 2001.
(60) Provisional application No. 60/194,241, filed on Apr. 3, 2000.
(60) Provisional application No. 60/219,862, filed on Jul. 20, 2000.
(60) Provisional application No. 60/221,300, filed on Jul. 27, 2000.
(60) Provisional application No. 60/256,592, filed on Dec. 18, 2000.

(51) Int. Cl. C12Q 1/68; C12P 19/34; G06F 19/00; G01N 33/48; G01N 33/50

(52) U.S. Cl. 435/6; 435/91.2; 702/20

(57) ABSTRACT

Compositions and methods for the therapy and diagnosis of cancer, such as breast cancer, are disclosed. Compositions may comprise one or more breast tumor proteins, immunogenic portions thereof, or polynucleotides that encode such portions. Alternatively, a therapeutic composition may comprise an antigen presenting cell that expresses a breast tumor protein, or a T cell that is specific for cells expressing such a protein. Such compositions may be used, for example, for the prevention and treatment of diseases such as breast cancer. Diagnostic methods based on detecting a breast tumor protein, or mRNA encoding such a protein, in a sample are also provided.

### REFERENCE TO RELATED APPLICATIONS

This application is a continuation-in-part of U.S. application Ser. No. 09/825,301, filed Apr. 2, 2001, which is incorporated herein by reference in its entirety.

### GOVERNMENTAL SUPPORT

This work was supported in part by Grants CA-75794 and CA-80518 from the National Cancer Institute. The government may have certain rights in the invention.

### TECHNICAL FIELD OF THE INVENTION

The present invention relates generally to the field of cancer diagnostics. More specifically, the present invention relates to methods, compositions and kits for the detection of cancer that employ oligonucleotide hybridization and/or amplification to simultaneously detect two or more tissue-specific polynucleotides in a biological sample suspected of containing cancer cells.

### BACKGROUND OF THE INVENTION

1. Field of the Invention

Cancer remains a significant health problem throughout the world. The failure of conventional cancer treatment regimens can commonly be attributed, in part, to delayed disease diagnosis. Although significant advances have been made in the area of cancer diagnosis, there still remains a need for improved detection methodologies that permit early, reliable and sensitive determination of the presence of cancer cells.

2. Description of the Related Art

Breast cancer is second only to lung cancer in mortality among women in the U.S., affecting more than 180,000 women each year and resulting in approximately 40,000-50,000 deaths annually. For women in North America, the life-time odds of getting breast cancer are one in eight.

Management of the disease currently relies on a combination of early diagnosis (through routine breast screening procedures) and aggressive treatment, which may include one or more of a variety of treatments such as surgery, radiotherapy, chemotherapy and hormone therapy. The course of treatment for a particular breast cancer is often selected based on a variety of prognostic parameters, including analysis of specific tumor markers. See, e.g., Porter-Jordan et al., Breast Cancer 8:73-100 (1994). The use of established markers often leads, however, to a result that is difficult to interpret; and the high mortality observed in breast cancer patients indicates that improvements are needed in the diagnosis of the disease.

The recent introduction of immunotherapeutic approaches to breast cancer treatment which are targeted to Her2/neu have provided significant motivation to identify additional breast cancer specific genes as targets for therapeutic antibodies and T-cell vaccines as well as for diagnosis of the disease. To this end, mammaglobin, has been identified as one of the most breast-specific genes discovered to date, being expressed in approximately 70-80% of breast cancers. Because of its highly tissue-specific distribution, detection of mammaglobin gene expression has been used to identify micrometastatic lesions in lymph node tissues and, more recently, to detect circulating breast cancer cells in peripheral blood of breast cancer patients with known primary and metastatic lesions.

Mammaglobin is a homologue of a rabbit uteroglobin and the rat steroid binding protein subunit C3 and is a low molecular weight protein that is highly glycosylated. Watson et al., Cancer Res. 56:860-5 (1996); Watson et al., Cancer Res. 59:3028-3031 (1999); Watson et al., Oncogene 16:817-24 (1998). In contrast to its homologs, mammaglobin has been reported to be breast specific and overexpression has been described in breast tumor biopsies (23%), primary and metastatic breast tumors (˜75%) with reports of the detection of mammaglobin mRNA expression in 91% of lymph nodes from metastatic breast cancer patients. Leygue et al., J. Pathol. 189:28-33 (1999) and Min et al., Cancer Res. 58:4581-4584 (1998).

Since mammaglobin gene expression is not a universal feature of breast cancer, the detection of this gene alone may be insufficient to permit the reliable detection of all breast cancers. Accordingly, what is needed in the art is a methodology that employs the detection of two or more breast cancer specific genes in order to improve the sensitivity and reliability of detection of micrometastases, for example, in lymph nodes and bone marrow and/or for recognition of anchorage-independent cells in the peripheral circulation.

The present invention achieves these and other related objectives by providing methods that are useful for the identification of tissue-specific polynucleotides, in particular tumor-specific polynucleotides, as well as methods, compositions and kits for the detection and monitoring of cancer cells in a patient afflicted with the disease.

### SUMMARY OF THE INVENTION

By certain embodiments, the present invention provides methods for identifying one or more tissue-specific polynucleotides which methods comprise the steps of: (a) performing a genetic subtraction to identify a pool of polynucleotides from a tissue of interest; (b) performing a DNA microarray analysis to identify a first subset of said pool of polynucleotides of interest wherein each member polynucleotide of said first subset is at least two-fold over-expressed in said tissue of interest as compared to a control tissue; and (c) performing a quantitative polymerase chain reaction analysis on polynucleotides within said first subset to identify a second subset of polynucleotides that are at least two-fold over-expressed as compared to the control tissue. Preferred genetic subtractions are selected from the group consisting of differential display and cDNA subtraction and are described in further detail herein below.

Alternate embodiments of the present invention provide methods of identifying a subset of polynucleotides showing concordant and/or complementary tissue-specific expression profiles in a tissue of interest. Such methods comprise the steps of, (a) performing an expression analysis selected from the group consisting of DNA microarray and quantitative PCR to identify a first polynucleotides that is at least two-fold over-expressed in a tissue of interest as compared to a control tissue; and (b) performing an expression analysis selected from the group consisting of DNA microarray and quantitative PCR to identify a first polynucleotides that is at least two-fold over-expressed in a tissue of interest as compared to a control tissue.

Further embodiments of the present invention provide methods for detecting the presence of a cancer cell in a patient. Such methods comprise the steps of: (a) obtaining a biological sample from the patient; (b) contacting the biological sample with a first oligonucleotide pair wherein the members of the first oligonucleotide pair hybridize, under moderately stringent conditions, to a first polynucleotide and the complement thereof, respectively; (c) contacting the biological sample with a second oligonucleotide pair wherein the members of the second oligonucleotide pair hybridize, under moderately stringent conditions, to a second polynucleotide and the complement thereof, respectively and wherein the first polynucleotide is unrelated in nucleotide sequence to the second polynucleotide; (d) amplifying the first polynucleotide and the second polynucleotide; and (e) detecting the amplified first polynucleotide and the amplified second polynucleotide; wherein the presence of the amplified first polynucleotide or amplified second polynucleotide indicates the presence of a cancer cell in the patient.

By some embodiments, detection of the amplified first and/or second polynucleotides may be preceded by a fractionation step such as, for example, gel electrophoresis. Alternatively or additionally, detection of the amplified first and/or second polynucleotides may be achieved by hybridization of a labeled oligonucleotide probe that hybridizes specifically, under moderately stringent conditions, to the first or second polynucleotide. Oligonucleotide labeling may be achieved by incorporating a radiolabeled nucleotide or by incorporating a fluorescent label.

In certain preferred embodiments, cells of a specific tissue type may be enriched from the biological sample prior to the steps of detection. Enrichment may be achieved by a methodology selected from the group consisting of cell capture and cell depletion. Exemplary cell capture methods include immunocapture and comprise the steps of: (a) adsorbing an antibody to a tissue-specific cell surface to cells said biological sample; (b) separating the antibody adsorbed tissue-specific cells from the remainder of the biological sample. Exemplary cell depletion may be achieved by cross-linking red cells and white cells followed by a subsequent fractionation step to remove the cross-linked cells.

Alternative embodiments of the present invention provide methods for determining the presence or absence of a cancer in a patient, comprising the steps of: (a) contacting a biological sample obtained from the patient with an oligonucleotide that hybridizes to a polynucleotide that encodes a breast tumor protein; (b) detecting in the sample a level of a polynucleotide (such as, for example, mRNA) that hybridizes to the oligonucleotide; and (c) comparing the level of polynucleotide that hybridizes to the oligonucleotide with a predetermined cut-off value, and therefrom determining the presence or absence of a cancer in the patient. Within certain embodiments, the amount of mRNA is detected via polymerase chain reaction using, for example, at least one oligonucleotide primer that hybridizes to a polynucleotide encoding a polypeptide as recited above, or a complement of such a polynucleotide. Within other embodiments, the amount of mRNA is detected using a hybridization technique, employing an oligonucleotide probe that hybridizes to a polynucleotide that encodes a polypeptide as recited above, or a complement of such a polynucleotide.

In related aspects, methods are provided for monitoring the progression of a cancer in a patient, comprising the steps of: (a) contacting a biological sample obtained from a patient with an oligonucleotide that hybridizes to a polynucleotide that encodes a breast tumor protein; (b) detecting in the sample an amount of a polynucleotide that hybridizes to the oligonucleotide; (c) repeating steps (a) and (b) using a biological sample obtained from the patient at a subsequent point in time; and (d) comparing the amount of polynucleotide detected in step (c) with the amount detected in step (b) and therefrom monitoring the progression of the cancer in the patient.

Certain embodiments of the present invention provide that the step of amplifying said first polynucleotide and said second polynucleotide is achieved by the polymerase chain reaction (PCR).

Within certain embodiments, the cancer cell to be detected may be selected from the group consisting of prostate cancer, breast cancer, colon cancer, ovarian cancer, lung cancer head & neck cancer, lymphoma, leukemia, melanoma, liver cancer, gastric cancer, kidney cancer, bladder cancer, pancreatic cancer and endometrial cancer. Still further embodiments of the present invention provide that the biological sample is selected from the group consisting of blood, a lymph node and bone marrow. The lymph node may be a sentinel lymph node.

Within specific embodiments of present invention it is provided that the first polynucleotide is selected from the group consisting of mammaglobin, lipophilin B, GABAπ (B899P), B726P, B511S, B533S, B305D and B311D. Other embodiments provide that the second polynucleotide is selected from the group consisting of mammaglobin, lipophilin B, GABAπ (B899P), B726P, B511S, B533S, B305D and B311D.

Alternate embodiments of the present invention provide methods for detecting the presence or absence of a cancer in a patient, comprising the steps of: (a) contacting a biological sample obtained from a patient with a first oligonucleotide that hybridizes to a polynucleotide selected from the group consisting of mammaglobin and lipophilin B; (b) contacting the biological sample with a second oligonucleotide that hybridizes to a polynucleotide sequence selected from the group consisting of GABAπ (B899P), B726P, B511S, B533S, B305D and B311D; (c) detecting in the sample an amount of a polynucleotide that hybridizes to at least one of the oligonucleotides; and (d) comparing the amount of polynucleotide that hybridizes to the oligonucleotide to a predetermined cut-off value, and therefrom determining the presence or absence of a cancer in the patient.

According to certain embodiments, oligonucleotides may be selected from those disclosed herein such as those presented in SEQ ID NO:33-72. By other embodiments, the amount of polynucleotide that hybridizes to the oligonucleotide is determined using a polymerase chain reaction. Alternatively, the amount of polynucleotide that hybridizes to the oligonucleotide may be determined using a hybridization assay.

Still other embodiments of the present invention provide methods for determining the presence or absence of a cancer cell in a patient, comprising the steps of: (a) contacting a biological sample obtained from a patient with a first oligonucleotide that hybridizes to a polynucleotide selected from the group consisting of a polynucleotide depicted in SEQ ID NO:73 and SEQ ID NO:74 or complement thereof, (b) contacting the biological sample with a second oligonucleotide that hybridizes to a polynucleotide depicted in SEQ ID NO:75 or complement thereof; (c) contacting the biological sample with a third oligonucleotide that hybridizes to a polynucleotide selected from the group consisting of a polynucleotide depicted in SEQ ID NO:1, SEQ ID NO:3, SEQ ID NO:5, SEQ ID NO:6 and SEQ ID NO:7 or complement thereof; (d) contacting the biological sample with a fourth oligonucleotide that hybridizes to a polynucleotide selected from the group consisting of a polynucleotide depicted in SEQ ID NO: 11 or complement thereof; (e) contacting the biological sample with a fifth oligonucleotide that hybridizes to a polynucleotide selected from the group consisting of a polynucleotide depicted in SEQ ID NO: 13, 15 and 17 or complement thereof; (f) contacting the biological sample with a sixth oligonucleotide that hybridizes to a polynucleotide selected from the group consisting of a polynucleotide depicted in SEQ ID NO:19, SEQ ID NO:20, SEQ ID NO:21, SEQ ID NO:22, SEQ ID NO:23 and SEQ ID NO:24 or complement thereof; (g) contacting the biological sample with a seventh oligonucleotide that hybridizes to a polynucleotide depicted in SEQ ID NO:30 or complement thereof; (h) contacting the biological sample with an eighth oligonucleotide that hybridizes to a polynucleotide depicted in SEQ ID NO:32 or complement thereof; (i) contacting the biological sample with a ninth oligonucleotide that hybridizes to a polynucleotide depicted in SEQ ID NO:76 or complement thereof; (j) detecting in the sample a hybridized oligonucleotide of any one of steps (a) through (i); and (j) comparing the amount of polynucleotide that hybridizes to the oligonucleotide to a predetermined cut-off value, wherein the presence of a hybridized oligonucleotide in any one of steps (a) through (i) in excess of the pre-determined cut-off value indicates the presence of a cancer cell in the biological sample of said patient.

Other related embodiments of the present invention provide methods for determining the presence or absence of a cancer cell in a patient, comprising the steps of: (a) contacting a biological sample obtained from a patient with a first oligonucleotide and a second oligonucleotide wherein said first and second oligonucleotides hybridize under moderately stringent conditions to a first and a second polynucleotide selected from the group selected from the group consisting of SEQ ID NO:73, SEQ ID NO:74, SEQ ID NO:75, SEQ ID NO:1, SEQ ID NO:3, SEQ ID NO:5, SEQ ID NO:6, SEQ ID NO:7, SEQ ID NO:11, SEQ ID NO:13, SEQ ID NO:15, SEQ ID NO:17, SEQ ID NO:19, SEQ ID NO:20, SEQ ID NO:21, SEQ ID NO:22, SEQ ID NO:23, SEQ ID NO:24, SEQ ID NO:30, SEQ ID NO:32, and SEQ ID NO:76 and wherein said first polynucleotide is unrelated structurally to said second polynucleotide; (b) detecting in the sample said first and said second hybridized oligonucleotides; and (c) comparing the amount of polynucleotide that hybridizes to the oligonucleotide to a predetermined cut-off value, wherein the presence of a hybridized first oligonucleotide or a hybridized second oligonucleotide in excess of the pre-determined cut-off value indicates the presence of a cancer cell in the biological sample of said patient.

Other related embodiments of the present invention provide methods for determining the presence or absence of a cancer cell in a patient, comprising the steps of: (a) contacting a biological sample obtained from a patient with a first oligonucleotide and a second oligonucleotide wherein said first and second oligonucleotides hybridize under moderately stringent conditions to a first and a second polynucleotide are both tissue-specific polynucleotides of the cancer to be detected and wherein said first polynucleotide is unrelated structurally to said second polynucleotide; (b) detecting in the sample said first and said second hybridized oligonucleotides; and (c) comparing the amount of polynucleotide that hybridizes to the oligonucleotide to a predetermined cut-off value, wherein the presence of a hybridized first oligonucleotide or a hybridized second oligonucleotide in excess of the pre-determined cut-off value indicates the presence of a cancer cell in the biological sample of said patient.

In other related aspects, the present invention further provides compositions useful in the methods disclosed herein. Exemplary compositions comprise two or more oligonucleotide primer pairs each one of which specifically hybridizes to a distinct polynucleotide. Exemplary oligonucleotide primers suitable for compositions of the present invention are disclosed herein by SEQ ID NO: 33-71. Exemplary polynucleotides suitable for compositions of the present invention are disclosed in SEQ ID NO:73, SEQ ID NO:74, SEQ ID NO:75, SEQ ID NO:1, SEQ ID NO:3, SEQ ID NO:5, SEQ ID NO:6, SEQ ID NO:7, SEQ ID NO:11, SEQ ID NO:13, SEQ ID NO:15, SEQ ID NO:17, SEQ ID NO:19, SEQ ID NO:20, SEQ ID NO:21, SEQ ID NO:22, SEQ ID NO:23, SEQ ID NO:24, SEQ ID NO:30, SEQ ID NO:32, and SEQ ID NO:76.

The present invention also provides kits that are suitable for performing the detection methods of the present invention. Exemplary kits comprise oligonucleotide primer pairs each one of which specifically hybridizes to a distinct polynucleotide. Within certain embodiments, kits according to the present invention may also comprise a nucleic acid polymerase and suitable buffer. Exemplary oligonucleotide primers suitable for kits of the present invention are disclosed herein by SEQ ID NO: 33-71. Exemplary polynucleotides suitable for kits of the present invention are disclosed in SEQ ID NO:73, SEQ ID NO:74, SEQ ID NO:75, SEQ ID NO:1, SEQ ID NO:3, SEQ ID NO:5, SEQ ID NO:6, SEQ ID NO:7, SEQ ID NO:11, SEQ ID NO:13, SEQ ID NO: 15, SEQ ID NO: 17, SEQ ID NO:19, SEQ ID NO:20, SEQ ID NO:21, SEQ ID NO:22, SEQ ID NO:23, SEQ ID NO:24, SEQ ID NO:30, SEQ ID NO:32, and SEQ ID NO:76.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS AND SEQUENCE IDENTIFIERS

FIG. 1 shows the mRNA expression profiles for B311D, B533S and B726P as determined using quantitative PCR (Taqman™). Abbreviations: B.T.: Breast tumor; B.M.: Bone marrow; B.R.: Breast reduction.

FIG. 2 shows the relationship of B533S expression to pathological stage of tumor. Tissues from normal breast (8), benign breast disorders (3), and breast tumors stage 1 (5), stage II (6), stage III (7), stage IV (3) and metastases (1 lymph node and 3 pleural effusions) were tested in real-time PCR. The data is expressed as the mean copies/ng β-actin for each group tested and the line is the calculated trend line.

FIGS. 3A and 3B show the gene complementation of B305D C-form, B726P, GABAπ and mammaglobin in metastases and primary tumors, respectively. The cut-off for each of the genes was 6.57, 1.65, 4.58 and 3.56 copies/ng β-Actin based on the mean of the negative normal tissues plus 3 standard deviations.

FIG. 4 shows the full-length cDNA sequence for mammaglobin.

FIG. 5 shows the determined cDNA sequence of the open reading frame encoding a mammaglobin recombinant polypeptide expressed in E. coli.

FIG. 6 shows the full-length cDNA sequence for GABAπ.

FIG. 7 shows the mRNA expression levels for mammaglobin, GABAπ, B305D (C form) and B726P in breast tumor and normal samples determined using real-time PCR and the SYBR detection system. Abbreviations: BT: Breast tumor; BR: Breast reduction; A. PBMC: Activated peripheral blood mononuclear cells; R. PBMC: resting PBMC; T. Gland: Thyroid gland; S. Cord: Spinal Cord; A. Gland: Adrenal gland; B. Marrow: Bone marrow; S. Muscle: Skeletal muscle.

FIG. 8 is a bar graph showing a comparison between the LipophilinB alone and the LipophilinB-B899P-B305D-C-B726 multiplex assays tested on a panel of breast tumor samples. Abbreviations: BT: Breast tumor; BR: Breast reduction; SCID: severe combined immunodeficiency.

FIG. 9 is a gel showing the unique band length of four amplification products of tumor genes of interest (mammaglobin, B305D, B899P, B726P) tested in a multiplex Real-time PCR assay.

FIG. 10 shows a comparison of a multiplex assay using intron-exon border spanning primers (bottom panel) and those using non-optimized primers (top panel), to detect breast cancer cells in a panel of lymph node tissues.

SEQ ID NO: 1 is the determined cDNA sequence for a first splice variant of B305D isoform A.

SEQ ID NO: 2 is the amino acid sequence encoded by the sequence of SEQ ID NO: 1.

SEQ ID NO: 3 is the determined cDNA sequence for a second splice variant of B305D isoform A.

SEQ ID NO: 4 is the amino acid sequence encoded by the sequence of SEQ ID NO: 3.

SEQ ID NO: 5-7 are the determined cDNA sequences for three splice variants of B305D isoform C.

SEQ ID NO: 8-10 are the amino acid sequences encoded by the sequence of SEQ ID NO: 5-7, respectively.

SEQ ID NO: 11 is the determined cDNA sequence for B311D.

SEQ ID NO: 12 is the amino acid sequence encoded by the sequence of SEQ ID NO: 11.

SEQ ID NO: 13 is the determined cDNA sequence of a first splice variant of B726P.

SEQ ID NO: 14 is the amino acid sequence encoded by the sequence of SEQ ID NO: 13.

SEQ ID NO: 15 is the determined cDNA sequence of a second splice variant of B726P.

SEQ ID NO: 16 is the amino acid sequence encoded by the sequence of SEQ ID NO: 15.

SEQ ID NO: 17 is the determined cDNA sequence of a third splice variant of B726P.

SEQ ID NO: 18 is the amino acid sequence encoded by the sequence of SEQ ID NO: 17.

SEQ ID NO: 19-24 are the determined cDNA sequences of further splice variants of B726P.

SEQ ID NO: 25-29 are the amino acid sequences encoded by SEQ ID NO: 19-24, respectively.

SEQ ID NO: 30 is the determined cDNA sequence for B511S.

SEQ ID NO: 31 is the amino acid sequence encoded by SEQ ID NO: 30.

SEQ ID NO: 32 is the determined cDNA sequence for B533S.

SEQ ID NO:33 is the DNA sequence of Lipophilin B forward primer.

SEQ ID NO:34 is the DNA sequence of Lipophilin B reverse primer.

SEQ ID NO:35 is the DNA sequence of Lipophilin B probe.

SEQ ID NO:36 is the DNA sequence of GABA (B899P) forward primer.

SEQ ID NO:37 is the DNA sequence of GABA (B899P) reverse primer.

SEQ ID NO:38 is the DNA sequence of GABA (B899P) probe.

SEQ ID NO:39 is the DNA sequence of B305D (C form) forward primer.

SEQ ID NO:40 is the DNA sequence of B305D (C form) reverse primer.

SEQ ID NO:41 is the DNA sequence of B305D (C form) probe.

SEQ ID NO:42 is the DNA sequence of B726P forward primer.

SEQ ID NO:43 is the DNA sequence of B726P reverse primer.

SEQ ID NO:44 is the DNA sequence of B726P probe.

SEQ ID NO:45 is the DNA sequence of Actin forward primer.

SEQ ID NO:46 is the DNA sequence of Actin reverse primer.

SEQ ID NO:47 is the DNA sequence of Actin probe.

SEQ ID NO:48 is the DNA sequence of Mammaglobin forward primer.

SEQ ID NO:49 is the DNA sequence of Mammaglobin reverse primer.

SEQ ID NO:50 is the DNA sequence of Mammaglobin probe.

SEQ ID NO:51 is the DNA sequence of a second GABA (B899P) reverse primer.

SEQ ID NO:52 is the DNA sequence of a second B726P forward primer.

SEQ ID NO:53 is the DNA sequence of a GABA B899P-INT forward primer.

SEQ ID NO:54 is the DNA sequence of a GABA B899P-INT reverse primer.

SEQ ID NO:55 is the DNA sequence of a GABA B899P-INT Taqman probe.

SEQ ID NO:56 is the DNA sequence of a B305D-INT forward primer.

SEQ ID NO:57 is the DNA sequence of a B305D-INT reverse primer.

SEQ ID NO:58 is the DNA sequence of a B305D-INT Taqman probe.

SEQ ID NO:59 is the DNA sequence of a B726-INT forward primer.

SEQ ID NO:60 is the DNA sequence of a B726-INT reverse primer.

SEQ ID NO:61 is the DNA sequence of a B726-INT Taqman probe.

SEQ ID NO:62 is the DNA sequence of a GABA B899P Taqman probe.

SEQ ID NO:63 is the DNA sequence of a B311D forward primer.

SEQ ID NO:64 is the DNA sequence of a B311D reverse primer.

SEQ ID NO:65 is the DNA sequence of a B311D Taqman probe.

SEQ ID NO:66 is the DNA sequence of a B533S forward primer.

SEQ ID NO:67 is the DNA sequence of a B533S reverse primer.

SEQ ID NO:68 is the DNA sequence of a B533S Taqman probe.

SEQ ID NO:69 is the DNA sequence of a B511S forward primer.

SEQ ID NO:70 is the DNA sequence of a B511S reverse primer.

SEQ ID NO:71 is the DNA sequence of a B511S Taqman probe.

SEQ ID NO:72 is the DNA sequence of a GABAπ reverse primer.

SEQ ID NO:73 is the full-length cDNA sequence for mammaglobin.

SEQ ID NO:74 is the determined cDNA sequence of the open reading frame encoding a mammaglobin recombinant polypeptide expressed in E. coli.

SEQ ID NO:75 is the full-length cDNA sequence for GABAπ.

SEQ ID NO:76 is the full-length cDNA sequence for lipophilin B.

SEQ ID NO:77 is the amino acid sequence encoded by the sequence of SEQ ID NO:76.

### DETAILED DESCRIPTION OF THE INVENTION

As noted above, the present invention is directed generally to methods that are suitable for the identification of tissue-specific polynucleotides as well as to methods, compositions and kits that are suitable for the diagnosis and monitoring of cancer. While certain exemplary methods, compositions and kits disclosed herein are directed to the identification, detection and monitoring of breast cancer, in particular breast cancer-specific polynucleotides, it will be understood by those of skill in the art that the present invention is generally applicable to the identification, detection and monitoring of a wide variety of cancers, and the associated over-expressed polynucleotides, including, for example, prostate cancer, breast cancer, colon cancer, ovarian cancer, lung cancer, head & neck cancer, lymphoma, leukemia, melanoma, liver cancer, gastric cancer, kidney cancer, bladder cancer, pancreatic cancer and endometrial cancer. Thus, it will be apparent that the present invention is not limited solely to the identification of breast cancer-specific polynucleotides or to the detection and monitoring of breast cancer.

Identification of Tissue-specific Polynucleotides

Certain embodiments of the present invention provide methods, compositions and kits for the detection of a cancer cell within a biological sample. These methods comprise the step of detecting one or more tissue-specific polynucleotide(s) from a patient's biological sample the over-expression of which polynucleotides indicates the presence of a cancer cell within the patient's biological sample. Accordingly, the present invention also provides methods that are suitable for the identification of tissue-specific polynucleotides. As used herein, the phrases “tissue-specific polynucleotides” or “tumor-specific polynucleotides” are meant to include all polynucleotides that are at least two-fold over-expressed as compared to one or more control tissues. As discussed in further detail herein below, over-expression of a given polynucleotide may be assessed, for example, by microarray and/or quantitative real-time polymerase chain reaction (Real-time PCR™) methodologies.

Exemplary methods for detecting tissue-specific polynucleotides may comprise the steps of: (a) performing a genetic subtraction to identify a pool of polynucleotides from a tissue of interest; (b) performing a DNA microarray analysis to identify a first subset of said pool of polynucleotides of interest wherein each member polynucleotide of said first subset is at least two-fold over-expressed in said tissue of interest as compared to a control tissue; and (c) performing a quantitative polymerase chain reaction analysis on polynucleotides within said first subset to identify a second subset of polynucleotides that are at least two-fold over-expressed as compared to said control tissue.

Polynucleotides Generally

As used herein, the term “polynucleotide” refers generally to either DNA or RNA molecules. Polynucleotides may be naturally occurring as normally found in a biological sample such as blood, serum, lymph node, bone marrow, sputum, urine and tumor biopsy samples. Alternatively, polynucleotides may be derived synthetically by, for example, a nucleic acid polymerization reaction. As will be recognized by the skilled artisan, polynucleotides may be single-stranded (coding or antisense) or double-stranded, and may be DNA (genomic, cDNA or synthetic) or RNA molecules. RNA molecules include mRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e. an endogenous sequence that encodes a tumor protein, such as a breast tumor protein, or a portion thereof) or may comprise a variant, or a biological or antigenic functional equivalent of such a sequence. Polynucleotide variants may contain one or more substitutions, additions, deletions and/or insertions, as further described below. The term “variants” also encompasses homologous genes of xenogenic origin.

When comparing polynucleotide or polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

Therefore, the present invention encompasses polynucleotide and polypeptide sequences having substantial identity to the sequences disclosed herein, for example those comprising at least 50% sequence identity, preferably at least 55%, 60%, 65%, 70%, 75%, 80%, 85%, 90%, 95%, 96%, 97%, 98%, or 99% or higher, sequence identity compared to a polynucleotide or polypeptide sequence of this invention using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

In additional embodiments, the present invention provides isolated polynucleotides and polypeptides comprising various lengths of contiguous stretches of sequence identical to or complementary to one or more of the sequences disclosed herein. For example, polynucleotides are provided by this invention that comprise at least about 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative DNA segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

In other embodiments, the present invention is directed to polynucleotides that are capable of hybridizing under moderately stringent conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

Moreover, it will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Microarray Analyses

Polynucleotides that are suitable for detection according to the methods of the present invention may be identified, as described in more detail below, by screening a microarray of cDNAs for tissue and/or tumor-associated expression (e.g., expression that is at least two-fold greater in a tumor than in normal tissue, as determined using a representative assay provided herein). Such screens may be performed, for example, using a Synteni microarray (Palo Alto, Calif.) according to the manufacturer's instructions (and essentially as described by Schena et al., Proc. Natl. Acad. Sci. USA 93:10614-10619 (1996) and Heller et al., Proc. Natl. Acad. Sci. USA 94:2150-2155 (1997)).

Microarray is an effective method for evaluating large numbers of genes but due to its limited sensitivity it may not accurately determine the absolute tissue distribution of low abundance genes or may underestimate the degree of overexpression of more abundant genes due to signal saturation. For those genes showing overexpression by microarray expression profiling, further analysis was performed using quantitative RT-PCR based on Taqman™ probe detection, which comprises a greater dynamic range of sensitivity. Several different panels of normal and tumor tissues, distant metastases and cell lines were used for this purpose.

Quantitative Real-time Polymerase Chain Reaction

Suitable polynucleotides according to the present invention may be further characterized or, alternatively, originally identified by employing a quantitative PCR methodology such as, for example, the Real-time PCR methodology. By this methodology, tissue and/or tumor samples, such as, e.g., metastatic tumor samples, may be tested along side the corresponding normal tissue sample and/or a panel of unrelated normal tissue samples.

Real-time PCR (see Gibson et al., Genome Research 6:995-1001, 1996; Heid et al., Genome Research 6:986-994, 1996) is a technique that evaluates the level of PCR product accumulation during amplification. This technique permits quantitative evaluation of mRNA levels in multiple samples. Briefly, mRNA is extracted from tumor and normal tissue and cDNA is prepared using standard techniques.

Real-time PCR may, for example, be performed either on the ABI 7700 Prism or on a GeneAmp® 5700 sequence detection system (PE Biosystems, Foster City, Calif.). The 7700 system uses a forward and a reverse primer in combination with a specific probe with a 5′ fluorescent reporter dye at one end and a 3′ quencher dye at the other end (Taqman™). When the Real-time PCR is performed using Taq DNA polymerase with 5′-3′ nuclease activity, the probe is cleaved and begins to fluoresce allowing the reaction to be monitored by the increase in fluorescence (Real-time). The 5700 system uses SYBR® green, a fluorescent dye, that only binds to double stranded DNA, and the same forward and reverse primers as the 7700 instrument. Matching primers and fluorescent probes may be designed according to the primer express program (PE Biosystems, Foster City, Calif.). Optimal concentrations of primers and probes are initially determined by those of ordinary skill in the art. Control (e.g., β-actin) primers and probes may be obtained commercially from, for example, Perkin Elmer/Applied Biosystems (Foster City, Calif.).

To quantitate the amount of specific RNA in a sample, a standard curve is generated using a plasmid containing the gene of interest. Standard curves are generated using the Ct values determined in the real-time PCR, which are related to the initial cDNA concentration used in the assay. Standard dilutions ranging from 10-10⁶ copies of the gene of interest are generally sufficient. In addition, a standard curve is generated for the control sequence. This permits standardization of initial RNA content of a tissue sample to the amount of control for comparison purposes.

In accordance with the above, and as described further below, the present invention provides the illustrative breast tissue- and/or tumor-specific polynucleotides mammaglobin, lipophilin B, GABAπ (B899P), B726P, B511S, B533S, B305D and B311D having sequences set forth in SEQ ID NO: 1, 3, 5-7, 11, 13, 15, 17, 19-24, 30, 32, and 73-76 illustrative polypeptides encoded thereby having amino acid sequences set forth in SEQ ID NO: 2, 4, 8-10, 12, 14, 16, 18, 25-29 and 31 and 77 that may be suitably employed in the detection of cancer, more specifically, breast cancer.

The methods disclosed herein will also permit the identification of additional and/or alternative polynucleotides that are suitable for the detection of a wide range of cancers including, but not limited to, prostate cancer, breast cancer, colon cancer, ovarian cancer, lung cancer head & neck cancer, lymphoma, leukemia, melanoma, liver cancer, gastric cancer, kidney cancer, bladder cancer, pancreatic cancer and endometrial cancer.

Methodologies for the Detection of Cancer

In general, a cancer cell may be detected in a patient based on the presence of one or more polynucleotides within cells of a biological sample (for example, blood, lymph nodes, bone marrow, sera, sputum, urine and/or tumor biopsies) obtained from the patient. In other words, such polynucleotides may be used as markers to indicate the presence or absence of a cancer such as, e.g., breast cancer.

As discussed in further detail herein, the present invention achieves these and other related objectives by providing a methodology for the simultaneous detection of more than one polynucleotide, the presence of which is diagnostic of the presence of cancer cells in a biological sample. Each of the various cancer detection methodologies disclosed herein have in common a step of hybridizing one or more oligonucleotide primers and/or probes, the hybridization of which is demonstrative of the presence of a tumor- and/or tissue-specific polynucleotide. Depending on the precise application contemplated, it may be preferred to employ one or more intron-spanning oligonucleotides that are inoperative against polynucleotide of genomic DNA and, thus, these oligonucleotides are effective in substantially reducing and/or eliminating the detection of genomic DNA in the biological sample.

Further disclosed herein are methods for enhancing the sensitivity of these detection methodologies by subjecting the biological samples to be tested to one or more cell capture and/or cell depletion methodologies.

By certain embodiments of the present invention, the presence of a cancer cell in a patient may be determined by employing the following steps: (a) obtaining a biological sample from said patient; (b) contacting said biological sample with a first oligonucleotide that hybridizes to a first polynucleotide said first polynucleotide selected from the group consisting of polynucleotides depicted in SEQ ID NO:73 and SEQ ID NO:74; (c) contacting said biological sample with a second oligonucleotide that hybridizes to a second polynucleotide selected from the group consisting of SEQ ID NO: 1, 3, 5-7, 11, 13, 15, 17, 19-24, 30, 32, and 75; (d) detecting in said sample an amount of a polynucleotide that hybridizes to at least one of the oligonucleotides; and (e) comparing the amount of the polynucleotide that hybridizes to said oligonucleotide to a predetermined cut-off value, and therefrom determining the presence or absence of a cancer in the patient.

Alternative embodiments of the present invention provide methods wherein the presence of a cancer cell in a patient is determined by employing the steps of: (a) obtaining a biological sample from said patient; (b) contacting said biological sample with a first oligonucleotide that hybridizes to a first polynucleotide said first polynucleotide depicted in SEQ ID NO:76; (c) contacting said biological sample with a second oligonucleotide that hybridizes to a second polynucleotide selected from the group consisting of SEQ ID NO: 1, 3, 5-7, 11, 13, 15, 17, 19-24, 30, 32, and 75; (d) detecting in said sample an amount of a polynucleotide that hybridizes to at least one of the oligonucleotides; and (e) comparing the amount of the polynucleotide that hybridizes to said oligonucleotide to a predetermined cut-off value, and therefrom determining the presence or absence of a cancer in the patient.

Other embodiments of the present invention provide methods for determining the presence or absence of a cancer in a patient. Such methods comprise the steps of: (a) obtaining a biological sample from said patient; (b) contacting said biological sample obtained from a patient with a first oligonucleotide that hybridizes to a polynucleotide sequence selected from the group consisting of polynucleotides depicted in SEQ ID NO:73, SEQ ID NO:74 and SEQ ID NO:76; (c) contacting said biological sample with a second oligonucleotide that hybridizes to a polynucleotide as depicted in SEQ ID NO:75; (d) contacting said biological sample with a third oligonucleotide that hybridizes to a polynucleotide selected from the group consisting of polynucleotides depicted in SEQ ID NO:5, SEQ ID NO:6 and SEQ ID NO:7; (e) contacting said biological sample with a fourth oligonucleotide that hybridizes to a polynucleotide selected from the group consisting of polynucleotides depicted in SEQ ID NO: 13, SEQ ID NO: 15, SEQ ID NO: 17, SEQ ID NO:19, SEQ ID NO:20, SEQ ID NO:21, SEQ ID NO:22, SEQ ID NO:23 and SEQ ID NO:24; (f) detecting in said biological sample an amount of a polynucleotide that hybridizes to at least one of said oligonucleotides; and (g) comparing the amount of polynucleotide that hybridizes to the oligonucleotide to a predetermined cut-off value, and therefrom determining the presence or absence of a cancer in the patient.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding a breast tumor protein that is at least 10 nucleotides, and preferably at least 20 nucleotides, in length. Preferably, oligonucleotide primers hybridize to a polynucleotide encoding a polypeptide described herein under moderately stringent conditions, as defined above. Oligonucleotide primers which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence recited in SEQ ID NO: 1, 3, 5-7, 11, 13, 15, 17, 19-24, 30, 32 and 73-76. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, N.Y., 1989).

The present invention also provides amplification-based methods for detecting the presence of a cancer cell in a patient. Exemplary methods comprise the steps of (a) obtaining a biological sample from a patient; (b) contacting the biological sample with a first oligonucleotide pair the first pair comprising a first oligonucleotide and a second oligonucleotide wherein the first oligonucleotide and the second oligonucleotide hybridize to a first polynucleotide and the complement thereof, respectively; (c) contacting the biological sample with a second oligonucleotide pair the second pair comprising a third oligonucleotide and a fourth oligonucleotide wherein the third and the fourth oligonucleotide hybridize to a second polynucleotide and the complement thereof, respectively, and wherein the first polynucleotide is unrelated in nucleotide sequence to the second polynucleotide; (d) amplifying the first polynucleotide and the second polynucleotide; and (e) detecting the amplified first polynucleotide and the amplified second polynucleotide; wherein the presence of the amplified first polynucleotide or the amplified second polynucleotide indicates the presence of a cancer cell in the patient.

Methods according to the present invention are suitable for identifying polynucleotides obtained from a wide variety of biological sample such as, for example, blood, serum, lymph node, bone marrow, sputum, urine and tumor biopsy sample. In certain preferred embodiments, the biological sample is either blood, a lymph node or bone marrow. In other embodiments of the present invention, the lymph node may be a sentinel lymph node.

It will be apparent that the present methods may be employed in the detection of a wide variety of cancers. Exemplary cancers include, but are not limited to, prostate cancer, breast cancer, colon cancer, ovarian cancer, lung cancer head & neck cancer, lymphoma, leukemia, melanoma, liver cancer, gastric cancer, kidney cancer, bladder cancer, pancreatic cancer and endometrial cancer.

Certain exemplary embodiments of the present invention provide methods wherein the polynucleotides to be detected are selected from the group consisting of mammaglobin, lipophilin B, GABAπ (B899P), B726P, B511S, B533S, B305D and B311D. Alternatively and/or additionally, polynucleotides to be detected may be selected from the group consisting of those depicted in SEQ ID NO:73, SEQ ID NO:74, SEQ ID NO:75, SEQ ID NO:1, SEQ ID NO:3, SEQ ID NO:5, SEQ ID NO:6, SEQ ID NO:7, SEQ ID NO: 11, SEQ ID NO:13, SEQ ID NO:15, SEQ ID NO:17, SEQ ID NO:19, SEQ ID NO:20, SEQ ID NO:21, SEQ ID NO:22, SEQ ID NO:23, SEQ ID NO:24, SEQ ID NO:30, SEQ ID NO:32, and SEQ ID NO:76.

Suitable exemplary oligonucleotide probes and/or primers that may be used according to the methods of the present invention are disclosed herein by SEQ ID NOs:33-35 and 63-72. In certain preferred embodiments that eliminate the background detection of genomic DNA, the oligonucleotides may be intron spanning oligonucleotides. Exemplary intron spanning oligonucleotides suitable for the detection of various polynucleotides disclosed herein are depicted in SEQ ID NO:36-62.

Depending on the precise application contemplated, the artisan may prefer to detect the tissue- and/or tumor-specific polynucleotides by detecting a radiolabel and detecting a fluorophore. More specifically, the oligonucleotide probe and/or primer may comprises a detectable moiety such as, for example, a radiolabel and/or a fluorophore.

Alternatively or additionally, methods of the present invention may also comprise a step of fractionation prior to detection of the tissue- and/or tumor-specific polynucleotides such as, for example, by gel electrophoresis.

In other embodiments, methods described herein may be used as to monitor the progression of cancer. By these embodiments, assays as provided for the diagnosis of a cancer may be performed over time, and the change in the level of reactive polypeptide(s) or polynucleotide(s) evaluated. For example, the assays may be performed every 24-72 hours for a period of 6 months to 1 year, and thereafter performed as needed. In general, a cancer is progressing in those patients in whom the level of polypeptide or polynucleotide detected increases over time. In contrast, the cancer is not progressing when the level of reactive polypeptide or polynucleotide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, polynucleotide probes may be used within such applications.

As noted above, to improve sensitivity, multiple breast tumor protein markers may be assayed within a given sample. It will be apparent that binding agents specific for different proteins provided herein may be combined within a single assay. Further, multiple primers or probes may be used concurrently. The selection of tumor protein markers may be based on routine experiments to determine combinations that results in optimal sensitivity. In addition, or alternatively, assays for tumor proteins provided herein may be combined with assays for other known tumor antigens.

Cell Enrichment

In other aspects of the present invention, cell capture technologies may be used prior to polynucleotide detection to improve the sensitivity of the various detection methodologies disclosed herein.

Exemplary cell enrichment methodologies employ immunomagnetic beads that are coated with specific monoclonal antibodies to surface cell markers, or tetrameric antibody complexes, may be used to first enrich or positively select cancer cells in a sample. Various commercially available kits may be used, including Dynabeads® Epithelial Enrich (Dynal Biotech, Oslo, Norway), StemSep™ (StemCell Technologies, Inc., Vancouver, BC), and RosetteSep (StemCell Technologies). The skilled artisan will recognize that other readily available methodologies and kits may also be suitably employed to enrich or positively select desired cell populations.

Dynabeads® Epithelial Enrich contains magnetic beads coated with mAbs specific for two glycoprotein membrane antigens expressed on normal and neoplastic epithelial tissues. The coated beads may be added to a sample and the sample then applied to a magnet, thereby capturing the cells bound to the beads. The unwanted cells are washed away and the magnetically isolated cells eluted from the beads and used in further analyses.

RosetteSep can be used to enrich cells directly from a blood sample and consists of a cocktail of tetrameric antibodies that target a variety of unwanted cells and crosslinks them to glycophorin A on red blood cells (RBC) present in the sample, forming rosettes. When centrifuged over Ficoll, targeted cells pellet along with the free RBC.

The combination of antibodies in the depletion cocktail determines which cells will be removed and consequently which cells will be recovered. Antibodies that are available include, but are not limited to: CD2, CD3, CD4, CD5, CD8, CD10, CD11b, CD14, CD15, CD16, CD19, CD20, CD24, CD25, CD29, CD33, CD34, CD36, CD38, CD41, CD45, CD45RA, CD45RO, CD56, CD66B, CD66e, HLA-DR, IgE, and TCRαβ. Additionally, it is contemplated in the present invention that mAbs specific for breast tumor antigens, can be developed and used in a similar manner. For example, mAbs that bind to tumor-specific cell surface antigens may be conjugated to magnetic beads, or formulated in a tetrameric antibody complex, and used to enrich or positively select metastatic breast tumor cells from a sample.

Once a sample is enriched or positively selected, cells may be further analysed. For example, the cells may be lysed and RNA isolated. RNA may then be subjected to RT-PCR analysis using breast tumor-specific multiplex primers in a Real-time PCR assay as described herein.

In another aspect of the present invention, cell capture technologies may be used in conjunction with Real-time PCR to provide a more sensitive tool for detection of metastatic cells expressing breast tumor antigens. Detection of breast cancer cells in bone marrow samples, peripheral blood, and small needle aspiration samples is desirable for diagnosis and prognosis in breast cancer patients.

Probes and Primers

As noted above and as described in further detail herein, certain methods, compositions and kits according to the present invention utilize two or more oligonucleotide primer pairs for the detection of cancer. The ability of such nucleic acid probes to specifically hybridize to a sequence of interest will enable them to be of use in detecting the presence of complementary sequences in a biological sample.

Alternatively, in other embodiments, the probes and/or primers of the present invention may be employed for detection via nucleic acid hybridization. As such, it is contemplated that nucleic acid segments that comprise a sequence region of at least about 15 nucleotide long contiguous sequence that has the same sequence as, or is complementary to, a 15 nucleotide long contiguous sequence of a polynucleotide to be detected will find particular utility. Longer contiguous identical or complementary sequences, e.g., those of about 20, 30, 40, 50, 100, 200, 500, 1000 (including all intermediate lengths) and even up to full length sequences will also be of use in certain embodiments.

Oligonucleotide primers having sequence regions consisting of contiguous nucleotide stretches of 10-14, 15-20, 30, 50, or even of 100-200 nucleotides or so (including intermediate lengths as well), identical or complementary to a polynucleotide to be detected , are particularly contemplated as primers for use in amplification reactions such as, e.g., the polymerase chain reaction (PCR™). This would allow a polynucleotide to be analyzed, both in diverse biological samples such as, for example, blood, lymph nodes and bone marrow.

The use of a primer of about 15-25 nucleotides in length allows the formation of a duplex molecule that is both stable and selective. Molecules having contiguous complementary sequences over stretches greater than 15 bases in length are generally preferred, though, in order to increase stability and selectivity of the hybrid, and thereby improve the quality and degree of specific hybrid molecules obtained. One will generally prefer to design primers having gene-complementary stretches of 15 to 25 contiguous nucleotides, or even longer where desired.

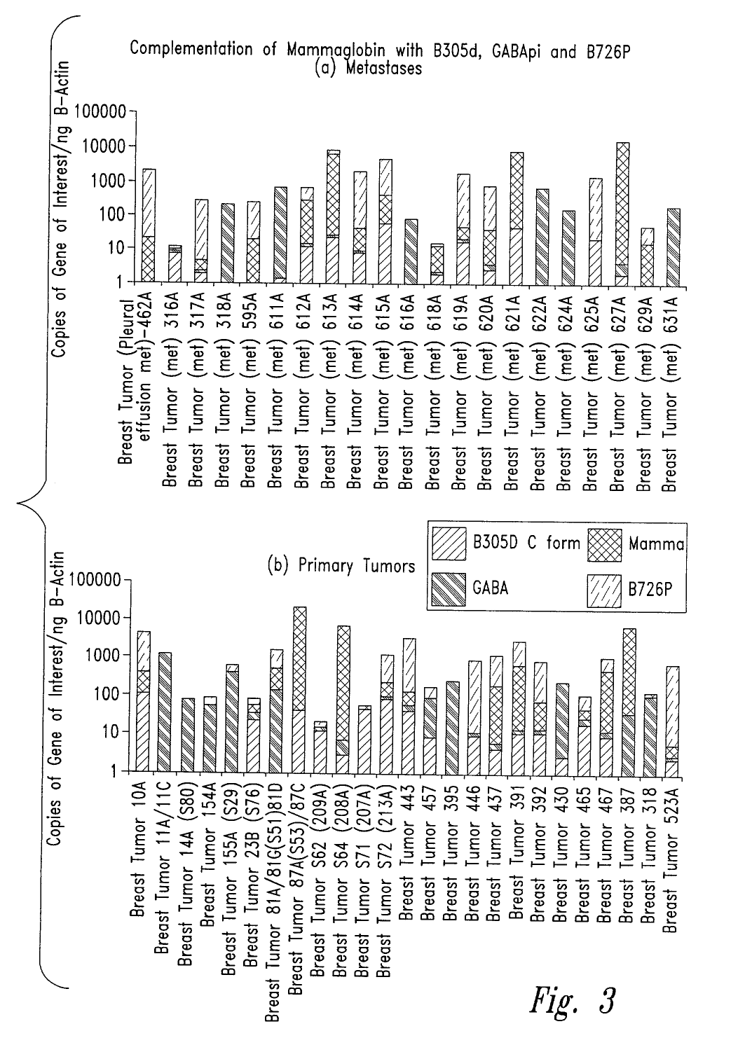

Primers may be selected from any portion of the polynucleotide to be detected. All that is required is to review the sequence, such as those exemplary polynucleotides set forth in SEQ ID NO: 1, 3, 5-7, 11, 13, 15, 17, 19-24, 30, 32, 73-75 (FIGS. 3-6, respectively) and SEQ ID NO:76 (lipophilin B) or to any continuous portion of the sequence, from about 15-25 nucleotides in length up to and including the full length sequence, that one wishes to utilize as a primer. The choice of primer sequences may be governed by various factors. For example, one may wish to employ primers from towards the termini of the total sequence. The exemplary primers disclosed herein may optionally be used for their ability to selectively form duplex molecules with complementary stretches of the entire polynucleotide of interest such as those set forth in SEQ ID NO: 1, 3, 5-7, 11, 13, 15, 17, 19-24, 30, 32, 73-75 (FIGS. 3-6, respectively), and SEQ ID NO:76 (lipophilin B).

The present invention further provides the nucleotide sequence of various exemplary oligonucleotide primers and probes, set forth in SEQ ID NO: 33-71, that may be used, as described in further detail herein, according to the methods of the present invention for the detection of cancer.

Oligonucleotide primers according to the present invention may be readily prepared routinely by methods commonly available to the skilled artisan including, for example, directly synthesizing the primers by chemical means, as is commonly practiced using an automated oligonucleotide synthesizer. Depending on the application envisioned, one will typically desire to employ varying conditions of hybridization to achieve varying degrees of selectivity of probe towards target sequence. For applications requiring high selectivity, one will typically desire to employ relatively stringent conditions to form the hybrids, e.g., one will select relatively low salt and/or high temperature conditions, such as provided by a salt concentration of from about 0.02 M to about 0.15 M salt at temperatures of from about 50° C. to about 70° C. Such selective conditions tolerate little, if any, mismatch between the probe and the template or target strand, and would be particularly suitable for isolating related sequences.

Polynucleotide Amplification Techniques

Each of the specific embodiments outlined herein for the detection of cancer has in common the detection of a tissue- and/or tumor-specific polynucleotide via the hybridization of one or more oligonucleotide primers and/or probes. Depending on such factors as the relative number of cancer cells present in the biological sample and/or the level of polynucleotide expression within each cancer cell, it may be preferred to perform an amplification step prior to performing the steps of detection. For example, at least two oligonucleotide primers may be employed in a polymerase chain reaction (PCR) based assay to amplify a portion of a breast tumor cDNA derived from a biological sample, wherein at least one of the oligonucleotide primers is specific for (i.e., hybridizes to) a polynucleotide encoding the breast tumor protein. The amplified cDNA may optionally be subjected to a fractionation step such as, for example, gel electrophoresis.

A number of template dependent processes are available to amplify the target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

One preferred methodology for polynucleotide amplification employs RT-PCR, in which PCR is applied in conjunction with reverse transcription. Typically, RNA is extracted from a biological sample, such as blood, serum, lymph node, bone marrow, sputum, urine and tumor biopsy samples, and is reverse transcribed to produce cDNA molecules. PCR amplification using at least one specific primer generates a cDNA molecule, which may be separated and visualized using, for example, gel electrophoresis. Amplification may be performed on biological samples taken from a patient and from an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

Any of a variety of commercially available kits may be used to perform the amplification step. One such amplification technique is inverse PCR (see Triglia et al., Nucl. Acids Res. 16:8186, 1988), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19, 1991) and walking PCR (Parker et al., Nucl. Acids. Res. 19:3055-60, 1991). Other methods employing amplification may also be employed to obtain a full length cDNA sequence.

Another method for amplification is the ligase chain reaction (referred to as LCR), disclosed in Eur. Pat. Appl. Publ. No. 320,308 (specifically incorporated herein by reference in its entirety). In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750, incorporated herein by reference in its entirety, describes an alternative method of amplification similar to LCR for binding probe pairs to a target sequence.

Qbeta Replicase, described in PCT Intl. Pat. Appl. Publ. No. PCT/US87/00880, incorporated herein by reference in its entirety, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[α-thio]triphosphates in one strand of a restriction site (Walker et al., 1992, incorporated herein by reference in its entirety), may also be useful in the amplification of nucleic acids in the present invention.

Strand Displacement Amplification (SDA) is another method of carrying out isothermal amplification of nucleic acids which involves multiple rounds of strand displacement and synthesis, i.e. nick translation. A similar method, called Repair Chain Reaction (RCR) is another method of amplification which may be useful in the present invention and is involves annealing several probes throughout a region targeted for amplification, followed by a repair reaction in which only two of the four bases are present. The other two bases can be added as biotinylated derivatives for easy detection. A similar approach is used in SDA.

Sequences can also be detected using a cyclic probe reaction (CPR). In CPR, a probe having a 3′ and 5′ sequences of non-target DNA and an internal or “middle” sequence of the target protein specific RNA is hybridized to DNA which is present in a sample. Upon hybridization, the reaction is treated with RNaseH, and the products of the probe are identified as distinctive products by generating a signal that is released after digestion. The original template is annealed to another cycling probe and the reaction is repeated. Thus, CPR involves amplifying a signal generated by hybridization of a probe to a target gene specific expressed nucleic acid.

Still other amplification methods described in Great Britain Pat. Appl. No. 2 202 328, and in PCT Intl. Pat. Appl. Publ. No. PCT/US89/01025, each of which is incorporated herein by reference in its entirety, may be used in accordance with the present invention. In the former application, “modified” primers are used in a PCR-like, template and enzyme dependent synthesis. The primers may be modified by labeling with a capture moiety (e.g., biotin) and/or a detector moiety (e.g., enzyme). In the latter application, an excess of labeled probes is added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS) (Kwoh et al., 1989; PCT Intl. Pat. Appl. Publ. No. WO 88/10315, incorporated herein by reference in its entirety), including nucleic acid sequence based amplification (NASBA) and 3SR. In NASBA, the nucleic acids can be prepared for amplification by standard phenol/chloroform extraction, heat denaturation of a sample, treatment with lysis buffer and minispin columns for isolation of DNA and RNA or guanidinium chloride extraction of RNA. These amplification techniques involve annealing a primer that has sequences specific to the target sequence. Following polymerization, DNA/RNA hybrids are digested with RNase H while double stranded DNA molecules are heat-denatured again. In either case the single stranded DNA is made fully double stranded by addition of second target-specific primer, followed by polymerization. The double stranded DNA molecules are then multiply transcribed by a polymerase such as T7 or SP6. In an isothermal cyclic reaction, the RNAs are reverse transcribed into DNA, and transcribed once again with a polymerase such as T7 or SP6. The resulting products, whether truncated or complete, indicate target-specific sequences.

Eur. Pat. Appl. Publ. No. 329,822, incorporated herein by reference in its entirety, disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from resulting DNA:RNA duplex by the action of ribonuclease H (RNase H, an RNase specific for RNA in a duplex with either DNA or RNA). The resultant ssDNA is a second template for a second primer, which also includes the sequences of an RNA polymerase promoter (exemplified by T7 RNA polymerase) 5′ to its homology to its template. This primer is then extended by DNA polymerase (exemplified by the large “Klenow” fragment of E. coli DNA polymerase I), resulting as a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

PCT Intl. Pat. Appl. Publ. No. WO 89/06700, incorporated herein by reference in its entirety, disclose a nucleic acid sequence amplification scheme based on the hybridization of a promoter/primer sequence to a target single-stranded DNA (“ssDNA”) followed by transcription of many RNA copies of the sequence. This scheme is not cyclic; i.e. new templates are not produced from the resultant RNA transcripts. Other amplification methods include “RACE” (Frohman, 1990), and “one-sided PCR” (Ohara, 1989) which are well-known to those of skill in the art.

Compositions and Kits for the Detection of Cancer

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to a breast tumor protein. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

The present invention also provides kits that are suitable for performing the detection methods of the present invention. Exemplary kits comprise oligonucleotide primer pairs each one of which specifically hybridizes to a distinct polynucleotide. Within certain embodiments, kits according to the present invention may also comprise a nucleic acid polymerase and suitable buffer. Exemplary oligonucleotide primers suitable for kits of the present invention are disclosed herein by SEQ ID NO: 33-71. Exemplary polynucleotides suitable for kits of the present invention are disclosed in SEQ ID NO:73, SEQ ID NO:74, SEQ ID NO:75, SEQ ID NO:1, SEQ ID NO:3, SEQ ID NO:5, SEQ ID NO:6, SEQ ID NO:7, SEQ ID NO:11, SEQ ID NO:13, SEQ ID NO:15, SEQ ID NO:17, SEQ ID NO:19, SEQ ID NO:20, SEQ ID NO:21, SEQ ID NO:22, SEQ ID NO:23, SEQ ID NO:24, SEQ ID NO:30, SEQ ID NO:32, and lipophilin B.

Alternatively, a kit may be designed to detect the level of mRNA encoding a breast tumor protein in a biological sample. Such kits generally comprise at least one oligonucleotide probe or primer, as described above, that hybridizes to a polynucleotide encoding a breast tumor protein. Such an oligonucleotide may be used, for example, within a PCR or hybridization assay. Additional components that may be present within such kits include a second oligonucleotide and/or a diagnostic reagent or container to facilitate the detection of a polynucleotide encoding a breast tumor protein.

In other related aspects, the present invention further provides compositions useful in the methods disclosed herein. Exemplary compositions comprise two or more oligonucleotide primer pairs each one of which specifically hybridizes to a distinct polynucleotide. Exemplary oligonucleotide primers suitable for compositions of the present invention are disclosed herein by SEQ ID NO: 33-71. Exemplary polynucleotides suitable for compositions of the present invention are disclosed in SEQ ID NO:73, SEQ ID NO:74, SEQ ID NO:75, SEQ ID NO:1, SEQ ID NO:3, SEQ ID NO:5, SEQ ID NO:6, SEQ ID NO:7, SEQ ID NO:11, SEQ ID NO:13, SEQ ID NO:15, SEQ ID NO:17, SEQ ID NO:19, SEQ ID NO:20, SEQ ID NO:21, SEQ ID NO:22, SEQ ID NO:23, SEQ ID NO:24, SEQ ID NO:30, SEQ ID NO:32, and lipophilin B.

The following Examples are offered by way of illustration and not by way of limitation.

### EXAMPLES

### Example 1

### Differential Display

This example discloses the use of differential display to enrich for polynucleotides that are over-expressed in breast tumor tissues.

Differential display was performed as described in the literature (see, e.g., Liang, P. et al., Science 257:967-971 (1993), incorporated herein by reference in its entirety) with the following modifications: (a) PCR amplification products were visualized on silver stained gels (b) genetically matched pairs of tissues were used to eliminate polymorphic variation (c) two different dilutions of cDNA were used as template to eliminate any dilutional effects (see, Mou, E. et al., Biochem Biophy Res Commun. 199:564-569 (1994), incorporated herein by reference in its entirety).

### Example 2

### Preparation of cDNA Subtraction Library

This example discloses the preparation of a breast tumor cDNA subtraction library enriched in breast tumor specific polynucleotides.

cDNA library subtraction was performed as described with some modification. See, Hara, T. et al., Blood 84: 189-199 (1994), incorporated herein by reference in its entirety. The breast tumor library (tracer) that was made from a pool of three breast tumors was subtracted with normal breast library (driver) to identify breast tumor specific genes. More recent subtractions utilized 6-10 normal tissues as driver to subtract out common genes more efficiently, with an emphasis on essential tissues along with one “immunological” tissue (e.g., spleen, lymph node, or PBMC), to assist in the removal of cDNAs derived from lymphocyte infiltration in tumors. The breast tumor specific subtracted cDNA library was generated as follows: driver cDNA library was digested with EcoRi, NotI, and SfuI (SfuI cleaves the vector), filled in with DNA polymerase klenow fragment. After phenol-chloroform extraction and ethanol precipitation, the DNA was labeled with Photoprobe biotin and dissolved in H₂O. Tracer cDNA library was digested with BamHI and XhoI, phenol chloroform extracted, passed through Chroma spin-400 columns, ethanol precipitated, and mixed with driver DNA for hybridization at 68° C. for 20 hours [long hybridization (LH)]. The reaction mixture was then subjected to the streptavidin treatment followed by phenol/chloroform extraction for a total of four times. Subtracted DNA was precipitated and subjected to a hybridization at 68° C. for 2 hours with driver DNA again [short hybridization (SH)]. After removal of biotinylated double-stranded DNA, subtracted cDNA was ligated into BamHI/XhoI site of Chloramphenicol resistant pBCSK+ and transformed into ElectroMax E. coli DH10B cells by electroporation to generate subtracted cDNA library. To clone less abundant breast tumor specific genes, cDNA library subtraction was repeated by subtracting the tracer cDNA library with the driver cDNA library plus abundant cDNAs from primary subtractions. This resulted in the depletion of these abundant sequences and the generation of subtraction libraries that contain less abundant sequences.

To analyze the subtracted cDNA library, plasmid DNA was prepared from 100-200 independent clones, which were randomly picked from the subtracted library, and characterized by DNA sequencing. The determined cDNA and expected amino acid sequences for the isolated cDNAs were compared to known sequences using the most recent Genbank and human EST databases.

### Example 3

### PCR-Subtraction

This example discloses PCR subtraction to enrich for breast tumor specific polynucleotides.

PCR-subtraction was performed essentially as described in the literature. See, Diatchenko, L. et al., Proc Natl Acad Sci USA. 93:6025-6030 (1996) and Yang, G. P. et al., Nucleic acids Res. 27:1517-23 (1999), incorporated herein by reference in their entirety. Briefly, this type of subtraction works by ligating two different adapters to different aliquots of a restriction enzyme digested tester (breast tumor) cDNA sample, followed by mixing of the testers separately with excess driver (without adapters). This first hybridization results in normalization of single stranded tester specific cDNA due to the second order kinetics of hybridization. These separate hybridization reactions are then mixed without denaturation, and a second hybridization performed which produces the target molecules; double stranded cDNA fragments containing both of the different adapters. Two rounds of PCR were performed, which results in the exponential amplification of the target population molecules (normalized tester specific cDNAs), while other fragments were either unamplified or only amplified in a linear manner. The subtractions performed included a pool of breast tumors subtracted with a pool of normal breast and a pool of breast tumors subtracted with a pool of normal tissues including PBMC, brain, pancreas, liver, small intestine, stomach, heart and kidney.

Prior to cDNA synthesis RNA was treated with DNase I (Ambion) in the presence of RNasin (Promega Biotech, Madison, Wis.) to remove DNA contamination. The cDNA for use in real-time PCR tissue panels was prepared using 25 μl Oligo dT (Boehringer-Mannheim) primer with superscript II reverse transcriptase (Gibco BRL, Bethesda, Md.).

### Example 4

### Detection of Breast Cancer Using Breast-Specific Antigens

The isolation and characterization of the breast-specific antigens B511S and B533S is described in U.S. patent application Ser. No. 09/346,327, filed Jul. 2, 1999, the disclosure of which is hereby incorporated by reference in its entirety. The determined cDNA sequence for B511S is provided in SEQ ID NO: 30, with the corresponding amino acid sequence being provided in SEQ ID NO: 31. The determined cDNA sequence for B533S is provided in SEQ ID NO: 32. The isolation and characterization of the breast-specific antigen B726P is described in U.S. patent application Ser. Nos. 09/285,480, filed Apr. 2, 1999, and 09/433,826, filed Nov. 3, 1999, the disclosures of which are hereby incorporated by reference in their entirety.

The determined cDNA sequences for splice variants of B726P are provided in SEQ ID NO: 13, 15, 17 and 19-24, with the corresponding amino acid sequences being provided in SEQ ID NO: 14, 16, 18 and 25-29.

The isolation and characterization of the breast-specific antigen B305D forms A and C has been described in U.S. patent application Ser. No. 09/429,755, filed Oct. 28, 1999, the disclosure of which is hereby incorporated by reference in its entirety. Determined cDNA sequences for B305D isoforms A and C are provided in SEQ ID NO: 1, 3 and 5-7, with the corresponding amino acid sequences being provided in SEQ ID NO: 2, 4and8-10.

The isolation and characterization of the breast-specific antigen B311D has been described in U.S. patent application Ser. No. 09/289,198, filed Apr. 9, 1999, the disclosure of which is hereby incorporated by reference in its entirety. The determined cDNA sequence for B311D is provided in SEQ ID NO:11, with the corresponding amino acid sequence being provided in SEQ ID NO: 12.

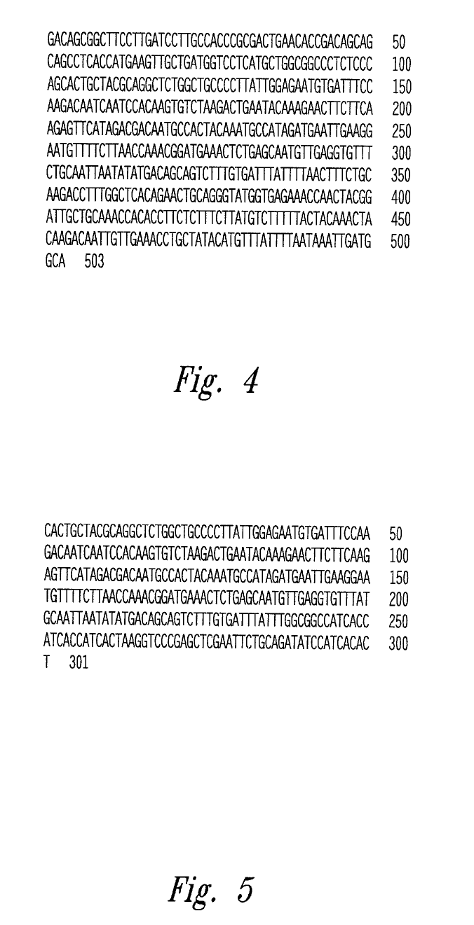

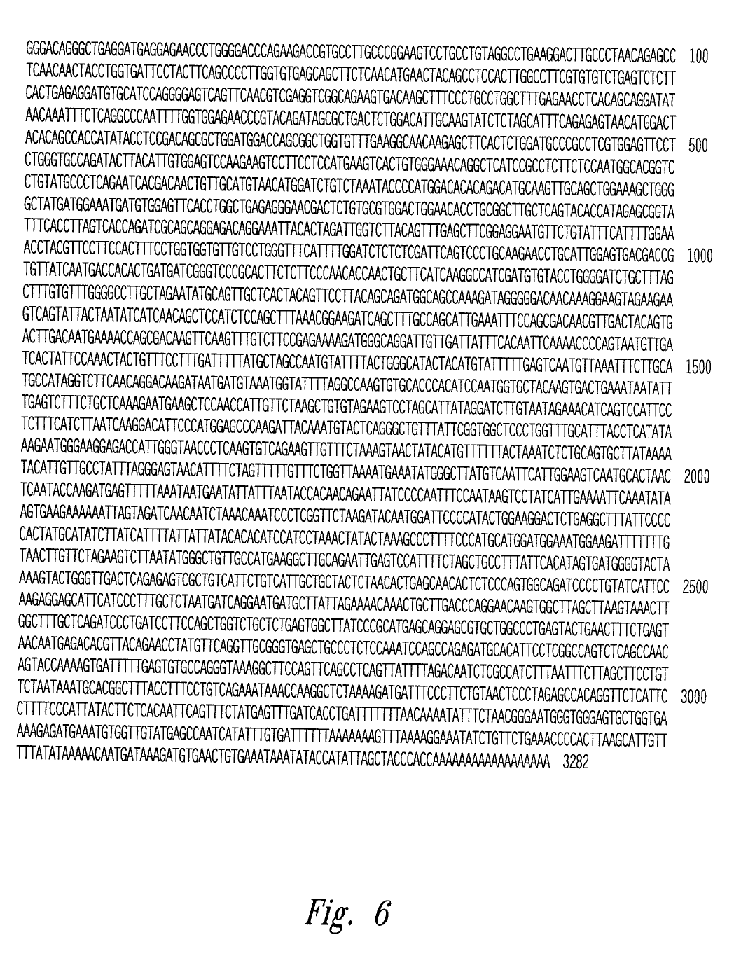

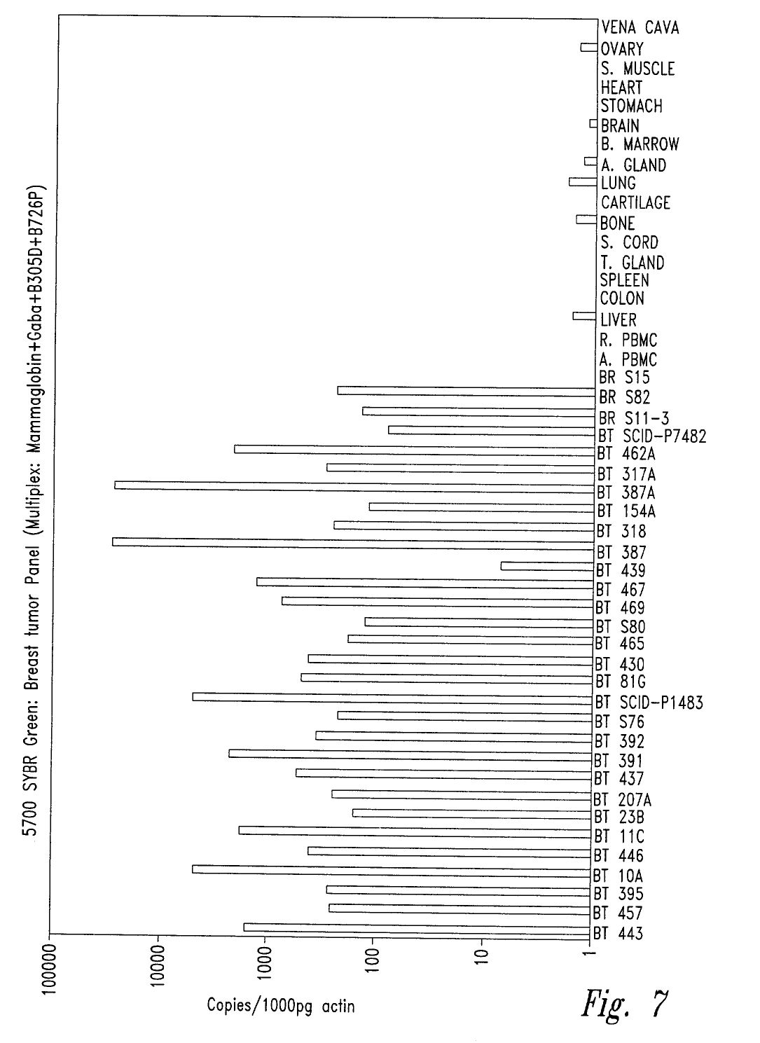

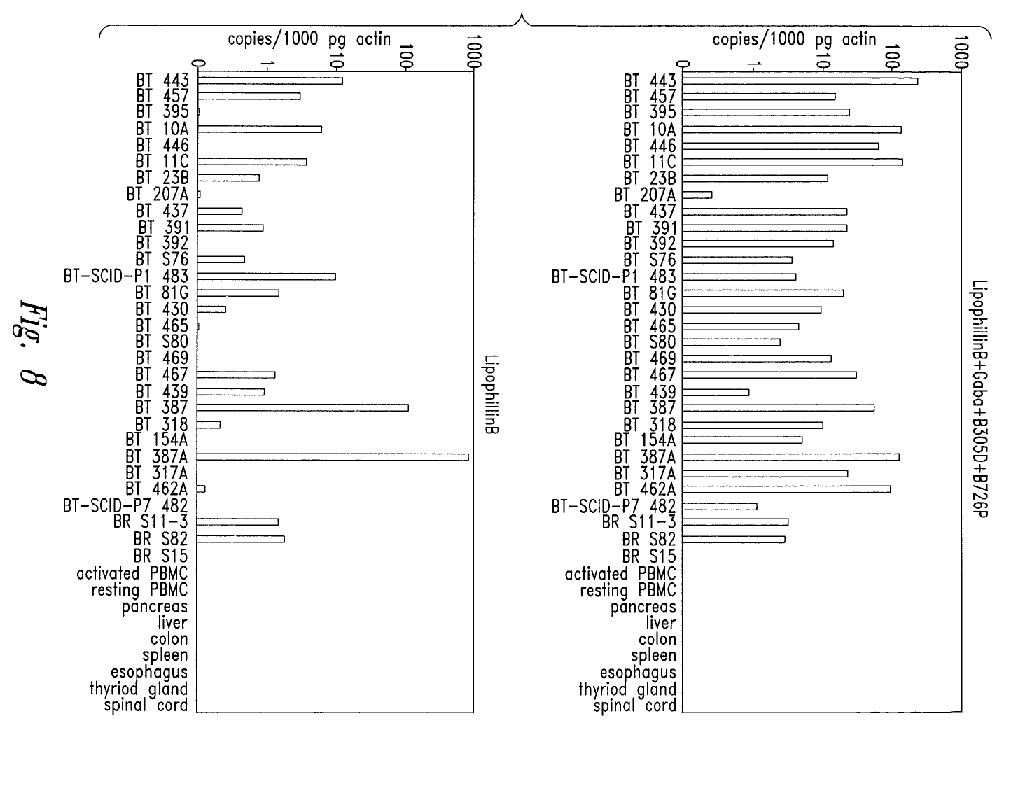

cDNA sequences for mammaglobin are provided in FIGS. 4 and 5, with the cDNA sequence for GABAπ being provided in FIG. 6 and are disclosed in SEQ ID NO: 73-75, respectively.

The isolation and characteization of the breast-specific antigen lipophilin B has been described in U.S. patent application Ser. No. 09/780,842, filed Feb. 8, 2001, the disclosure of which is hereby incorporated by reference in its entirety. The determined cDNA sequence for lipophilin B is provided in SEQ ID NO:76, with the corresponding amino acid sequence being provided in SEQ ID NO:77. The nucleotide sequences of several sequence variants of lipophilin B are also described in the 09/780,842 application.

### Example 5

### Microarray Analysis

This example discloses the use of microarray analyses to identify polynucleotides that are at least two-fold overexpressed in breast tumor tissue samples as compared to normal breast tissue samples.

mRNA expression of the polynucleotides of interest was performed as follows. cDNA for the different genes was prepared as described above and arrayed on a glass slide (Incyte, Palo Alto, Calif.). The arrayed cDNA was then hybridized with a 1:1 mixture of Cy3 or Cy5 fluorescent labeled first strand cDNAs obtained from polyA+ RNA from breast tumors, normal breast and normal tissues and other tumors as described in Shalon, D. et al., Genome Res. 6:639-45 (1996), incorporated herein by reference in its entirety. Typically Cy3 (Probe 1) was attached to cDNAs from breast tumors and Cy5 (Probe 2) to normal breast tissue or other normal tissues. Both probes were allowed to compete with the immobilized gene specific cDNAs on the chip, washed then scanned for fluorescence intensity of the individual Cy3 and Cy5 fluorescence to determine extent of hybridization. Data were analyzed using GEMTOOLS software (Incyte, Palo Alto, Calif.) which enabled the overexpression patterns of breast tumors to be compared with normal tissues by the ratios of Cy3/Cy5. The fluorescence intensity was also related to the expression level of the individual genes.

DNA microarray analyses was used primarily as a screening tool to determine tissue/tumor specificity of cDNA's recovered from the differential display, cDNA library and PCR subtractions, prior to more rigorous analysis by quantitative RT-PCR, northern blotting, and immunohistochemistry. Microarray analysis was performed on two microchips. A total of 3603 subtracted cDNA's and 197 differential display templates were evaluated to identify 40 candidates for further analysis by quantitative PCR. From these candidates, several were chosen on the basis of favorable tissue specificity profiles, including B305D, B311D, B726P, B511S and B533S, indicating their overexpression profiles in breast tumors and/or normal breast versus other normal tissues. It was evident that the expression of these genes showed a high degree of specificity for breast tumors and/or breast tissue. In addition, these genes have in many cases complementary expression profiles.

The two known breast-specific genes, mammaglobin and γ-aminobutyrate type A receptor π subunit (GABAπ) were also subjected to microarray analysis. mRNA expression of mammaglobin has been previously described to be upregulated in proliferating breast tissue, including breast tumors. See, (Watson et al., Cancer Res., 56: 860-5 (1996); Watson et al., Cancer Res., 59: 3028-3031 (1999); Watson et al., Oncogene. 16:817-24 (1998), incorporated herein by reference in their entirety). The GABAπ mRNA levels were over-expressed in breast tumors. Previous studies had demonstrated its overexpression in uterus and to some degree in prostate and lung (Hedblom et al., J Biol. Chem. 272:15346-15350 (1997)) but no previous study had indicated elevated levels in breast tumors.

### Example 6

### Quantitative Real-Time PCR Analysis

This example discloses the use of quantitative Real-time PCR to confirm the microarray identification polynucleotide that are at least two-fold overexpressed in breast tumor tissue samples as compared to normal breast tissue samples.

The tumor- and/or tissue-specificity of the polynucleotides identified by the microarray analyses disclosed herein in Example 5, were confirmed by quantitative PCR analyses. Breast metastases, breast tumors, benign breast disorders and normal breast tissue along with other normal tissues and tumors were tested in quantitative (Real time) PCR. This was performed either on the ABI 7700 Prism or on a GeneAmp® 5700 sequence detection system (PE Biosystems, Foster City, Calif.). The 7700 system uses a forward and a reverse primer in combination with a specific probe designed to anneal to sequence between the forward and reverse primer. This probe was conjugated at the 5′ end with a fluorescent reporter dye and a quencher dye at the other 3′ end (Taqman™). During PCR the Taq DNA polymerase with it's 5′-3′ nuclease activity cleaved the probe which began to fluoresce, allowing the reaction to be monitored by the increase in fluorescence (Real-time). Holland et al., Proc Natl Acad Sci USA. 88:7276-7280 (1991). The 5700 system used SYBR® green, a fluorescent dye, that only binds to double stranded DNA (Schneeberger et al., PCR Methods Appl. 4:234-8 (1995)), and the same forward and reverse primers as the 7700 instrument. No probe was needed. Matching primers and fluorescent probes were designed for each of the genes according to the Primer Express program (PE Biosystems, Foster City, Calif.).

TABLE 1
Primer and Probe Sequences for the Genes of Interest

| | Forward Primer | Reverse primer | Probe |
|---|---|---|---|
| Mammaglobin | TGCCATAGATGA | TGTCATATATTAATT | TCTTAACCAAACGG |
| | ATTGAAGGAATG | GCATAAACACCTCA | ATGAAACTCTGAGC |
| | (SEQ ID NO:48) | (SEQ ID NO:49) | (SEQ ID NO:50) |
| B305D-C form | AAAGCAGATGGT | CCTGAGACCAAATG | ATTCCATGCCGGCT |
| | GGTTGAGGTT | GCTTCTTC | GCTTCTTCTG |
| | (SEQ ID NO:39) | (SEQ ID NO:40) | (SEQ ID NO:41) |
| B311D | CCGCTTCTGACAA | CCTATAAAGATGTT | CCCCTCCCTCAGGG |
| | CACTAGAGATC | ATGTACCAAAAATG | TATGGCCC |
| | (SEQ ID NO:63) | AAGT (SEQ ID NO:64) | (SEQ ID NO:65) |
| B726P | TCTGGTTTTCTCA | TGCCAAGGAGCGGA | CAACCACGTGACA |
| | TTCTTTATTCATT | TTATCT | AACACTGGAATTAC |
| | TATT | (SEQ ID NO:43) | AGG (SEQ ID NO:44) |
| | (SEQ ID NO:42) | | |
| B533S | CCCTTTCTCACCC | TGCATTCTCTCATAT | CCGGGCCTCAGGC |
| | ACACACTGT | GTGGAAGCT | ATATACTATTCTAC |
| | (SEQ ID NO:66) | (SEQ ID NO:67) | TGTCTG |
| | | | (SEQ ID NO:68) |
| GABAπ | AAGCCTCAGAGT | AAATATAAGTGAAG | AATCCATTGTATCT |
| | CCTTCCAGTATG | AAAAAAATTAGTAG | TAGAACCGAGGGA |
| | (SEQ ID NO:36) | AT (SEQ ID NO:72) | TTTGTTTAGA |
| | | | (SEQ ID NO:38) |
| B511S | GACATTCCAGTTT | TGCAGAAGACTCAA | TCTCAGGGACACAC |
| | TACCCAAATGG | GCTGATTCC | TCTACCATTCGGGA |
| | (SEQ ID NO:69) | (SEQ ID NO:70) | (SEQ ID NO:71) |

The concentrations used in the quantitative PCR for the forward primers for mammaglobin, GABAπ, B305D C form, B311D, B511S, B533S and B726P were 900, 900, 300, 900, 900, 300 and 300 nM respectively. For the reverse primers they were 300, 900, 900, 900, 300, 900 and 900 nM respectively. Primers and probes so produced were used in the universal thermal cycling program in real-time PCR. They were titrated to determine the optimal concentrations using a checkerboard approach. A pool of cDNA from target tumors was used in this optimization process. The reaction was performed in 25 μl volumes. The final probe concentration in all cases was 160 nM. dATP, dCTP and dGTP were at 0.2 mM and dUTP at 0.4 mM. Amplitaq gold and Amperase UNG (PE Biosystems, Foster City, Calif.) were used at 0.625 units and 0.25 units per reaction. MgCl₂ was at a final concentration of 5 mM. Trace amounts of glycerol, gelatin and Tween 20 (Sigma Chem Co, St Louis, Mo.) were added to stabilize the reaction. Each reaction contained 2 μl of diluted template. The cDNA from RT reactions prepared as above was diluted 1:10 for the gene of interest and 1:100 for β-Actin. Primers and probes for β-Actin (PE Biosystems, Foster City, Calif.) were used in a similar manner to quantitate the presence of β-actin in the samples. In the case of the SYBR® green assay, the reaction mix (25 μl) included 2.5 μl of SYBR green buffer, 2 μl of cDNA template and 2.5 μl each of the forward and reverse primers for the gene of interest. This mix also contained 3 mM MgCl₂, 0.25 units of AmpErase UNG, 0.625 units of Amplitaq gold, 0.08% glycerol, 0.05% gelatin, 0.0001% Tween 20 and 1 mM dNTP mix. In both formats, 40 cycles of amplification were performed.

In order to quantitate the amount of specific cDNA (and hence initial mRNA) in the sample, a standard curve was generated for each run using the plasmid containing the gene of interest. Standard curves were generated using the Ct values determined in the real-time PCR which were related to the initial cDNA concentration used in the assay. Standard dilutions ranging from 20-2×10⁶ copies of the gene of interest were used for this purpose. In addition, a standard curve was generated for the housekeeping gene β-actin ranging from 200 fg-200 pg to enable normalization to a constant amount of β-Actin. This allowed the evaluation of the over-expression levels seen with each of the genes.

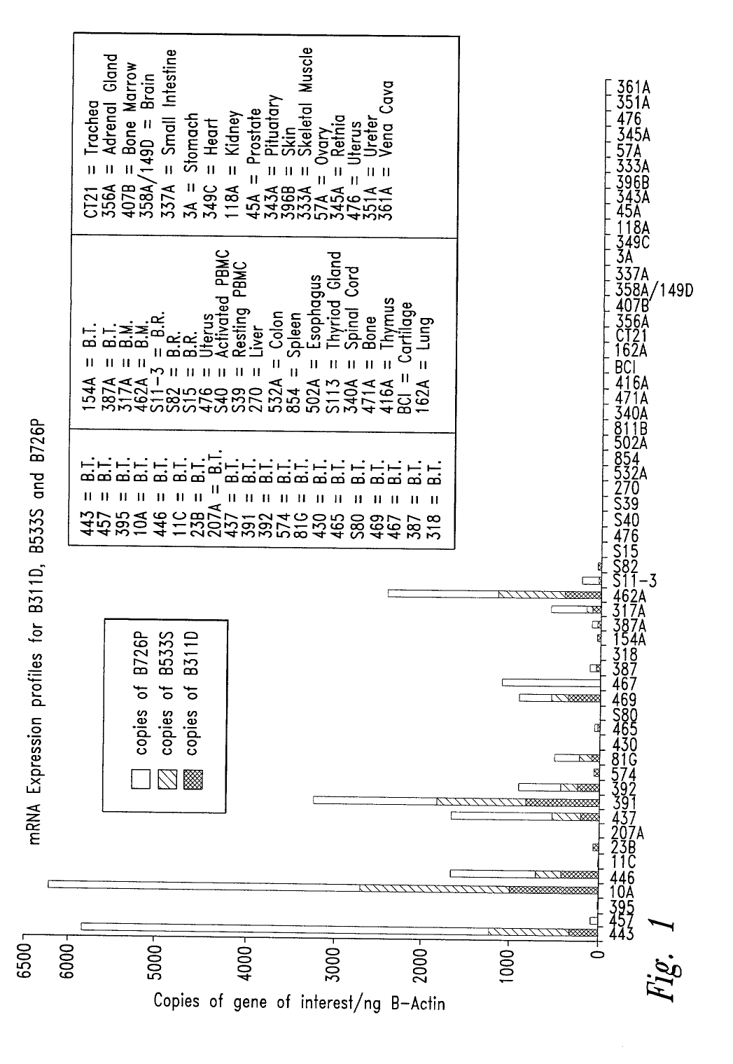

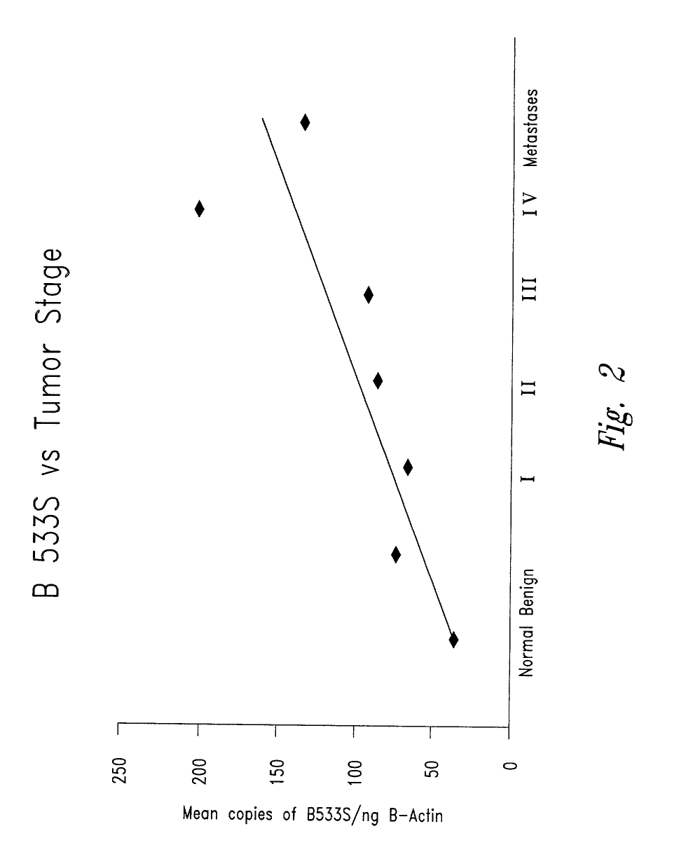

The genes B311D, B533S and B726P were evaluated in quantitative PCR as described above on two different panels consisting of: (a) breast tumor, breast normal and normal tissues; and (b) breast tumor metastases (primarily lymph nodes), using the primers and probes shown above in Table 1. The data for panel (a) is shown in FIG. 1 for all three genes. The three genes showed identical breast tissue expression profiles. However, the relative level of gene expression was very different in each case. B311D in general was expressed at lower levels than B533S and both less than B726P, but all three were restricted to breast tissue. The quantitative PCR thus confirmed there was a differential expression between normal breast tissue and breast tumors for all three genes, and that approximately 50% of breast tumors over-expressed these genes. When tested on a panel of distant metastases derived from breast cancers all three genes reacted with 14/21 metastases and presented similar profiles. All three genes also exhibited increasing levels of expression as a function of pathological stage of the tumor, as shown for B533S in FIG. 2.

Mammaglobin is a homologue of a rabbit uteroglobin and the rat steroid binding protein subunit C3 and is a low molecular weight protein that is highly glycosylated. In contrast to its homologs, mammaglobin has been reported to be breast specific and over-expression has been described in breast tumor biopsies (23%) and primary and metastatic breast tumors (˜75%) with reports of the detection of mammaglobin mRNA expression in 91% of lymph nodes from metastatic breast cancer patients. However, more rigorous analysis of mammaglobin gene expression by microarray and quantitative PCR as described above (panels (a) and (b) and a panel of other tumors and normal tissues and additional breast tumors), showed expression at significant levels in skin and salivary gland with much lower levels in esophagus and trachea, as shown in Table 2 below.

TABLE 2
Normalized Distribution of Mammaglobin and B511S mRNA in Various Tissues

| | Mean Copies | | | | |
|---|---|---|---|---|---|
| | Mammaglobin/ | | Mean Copies | | PCR Positive |
| | ng β-Actin ± | PCR | B511S/ | PCR | (Mammaglobin/ |
| Tissue | SD | Positive | ng β-Actin ± SD | Positive | B511S) |
| Breast | 1233.88 ± 3612.74 | 31/42 | 1800.40 ± 3893.24 | 33/42 | 38/42 |
| Tumors | | | | | |
| Breast | 1912.54 ± 4625.85 | 14/24 | 3329.50 ± 10820.71 | 14/24 | 17/24 |
| tumor | | | | | |
| Metastases | | | | | |
| Benign | 121.87 ± 78.63 | 3/3 | 524.66 ± 609.43 | 2/3 | 3/3 |
| Breast | | | | | |
| disorders | | | | | |
| Normal | 114.19 ± 94.40 | 11/11 | 517.64 ± 376.83 | 8/9 | 11/11 |
| breast | | | | | |
| Breast | 231.50 ± 276.68 | 2/3 | 482.54 ± 680.28 | ½ | 2/3 |
| reduction | | | | | |
| Other | 0.13 ± 0.65 | 1/39 | 24.17 ± 36.00 | 5/23 | |
| tumors | | | | | |
| Salivary | 435.65 ± 705.11 | 2/3 | 45766.61 ± 44342.43 | 3/3 | |
| gland | | | | | |
| Skin | 415.74 ± 376.14 | 7/9 | 7039.05 ± 7774.24 | 9/9 | |
| Esophagus | 4.45 ± 3.86 | 2/3 | 1.02 ± 0.14 | 0/3 | |
| Bronchia | 0.16 | 0/1 | 84.44 ± 53.31 | 2/2 | |
| Other | 0.33 ± 1.07 | 0/85 | 5.49 ± 10.65 | 3/75 | |
| normal | | | | | |
| tissues | | | | | |

The breast-specific gene B511S, while having a different profile of reactivity on breast tumors and normal breast tissue to mammaglobin, reacted with the same subset of normal tissues as mammaglobin. B511S by PSORT analysis is indicated to have an ORF of 90 aa and to be a secreted protein as is the case for mammaglobin. B511S has no evidence of a transmembrane domain but may harbor a cleavable signal sequence. Mammaglobin detected 14/24 of distant metastatic breast tumors, 31/42 breast tumors and exhibited ten-fold over-expression in tumors and metastases as compared to normal breast tissue. There was at least 300-fold over-expression in normal breast tissue versus other negative normal tissues and tumors tested, which were essentially negative for mammaglobin expression. B511S detected 33/42 breast tumors and 14/24 distant metastases, while a combination of B511S with mammaglobin would be predicted to detect 38/42 breast tumors and 17/24 metastatic lesions (Table 2 above). The quantitative level of expression of B511S and mammaglobin were also in similar ranges, in concordance with the microarray profiles observed for these two genes. Other genes that were additive with mammaglobin are shown in Table 3.

TABLE 3
mRNA Complementation of Mammaglobin with Other Genes

| | | Mammaglobin Negative | | | | |
|---|---|---|---|---|---|---|
| | Mammaglobin | | | | B305D + | B305D + GABAπ + |
| | Positive | B305D | GABAπ | B726P | GABAπ | B726P |
| Breast | 13/21 | 2/8 | 5/8 | 3/8 | 7/8 | 8/8 |
| Metastases | | | | | | |
| Breast | 18/25 | 3/7 | 4/7 | 5/7 | 7/7 | 7/7 |
| tumors | | | | | | |

B305D was shown to be highly over-expressed in breast tumors, prostate tumors, normal prostate tissue and testis compared to normal tissues, including normal breast tissue. Different splice variants of B305D have been identified with form A and C being the most abundant but all tested have similar tissue profiles in quantitative PCR. The A and C forms contain ORF's of 320 and 385 aa, respectively. B305D is predicted by PSORT to be a Type II membrane protein that comprises a series of ankyrin repeats. A known gene shown to be complementary with B305D, in breast tumors, was GABAπ. This gene is a member of the GABA_{A} receptor family and encodes a protein that has 30-40% amino acid homology with other family members, and has been shown by Northern blot analysis to be over-expressed in lung, thymus and prostate at low levels and highly over-expressed in uterus. Its expression in breast tissue has not been previously described. This is in contrast to other GABA_{A} receptors that have appreciable expression in neuronal tissues. Tissue expression profiling of this gene showed it to be over-expressed in breast tumors in an inverse relationship to the B305D gene (Table 3). GABAπ detected 15/25 tumors and 6/21 metastases including 4 tumors and 5 metastases missed by mammaglobin. In contrast, B305D detected 13/25 breast tumors and 8/21 metastases, again including 3 tumors and 2 metastases missed by mammaglobin. A combination of just B305D and the GABAπ would be predicted to identify 22/25 breast tumors and 14/21 metastases. The combination of B305D and GABAπ with mammaglobin in detecting breast metastases is shown in Table 3 above and FIGS. 3A and 3B. This combination detected 20/21 of the breast metastases as well as 25/25 breast tumors that were evaluated on the same panels for all three genes. The one breast metastasis that was negative for these three genes was strongly positive for B726P (FIGS. 3A and 3B).

To evaluate the presence of circulating tumor cells, an immunocapture (cell capture) method was employed to first enrich for epithelial cells prior to RT-PCR analysis. Immunomagnetic polystyrene beads coated with specific monoclonal antibodies to two glycoproteins on the surface of epithelial cells were used for this purpose. Such an enrichment procedure increased the sensitivity of detection (˜100 fold) as compared to direct isolation of poly A⁺RNA, as shown in Table 4.

TABLE 4
Extraction of Mammaglobin Positive Cells (MB415) Spiked
into Whole Blood and Detection by Real-time PCR

| | Epithelial cell extraction | Direct Extraction |
|---|---|---|
| | (Poly A⁺RNA) | (Poly A⁺RNA) |
| MB415 cells/ml Blood | Copies Mammaglobin/ng β-Actin | |
| 100000 | 54303.2 | 58527.1 |
| 10000 | 45761.9 | 925.9 |
| 1000 | 15421.2 | 61.6 |
| 100 | 368.0 | 5.1 |
| 10 | 282.0 | 1.1 |
| 1 | 110.2 | 0 |
| 0 | 0 | 0 |

Mannaglobin-positive cells (MB415) were spiked into whole blood at various concentrations and then extracted using either epithelial cell enrichment or direct isolation from blood. Using enrichment procedures, mammaglobin mRNA was found to be detectable at much lower levels than when direct isolation was used. Whole blood samples from patients with metatastic breast cancer were subsequently treated with the immunomagnetic beads. Poly A⁺ RNA was then isolated, cDNA prepared and run in quantitative PCR using two gene specific primers (Table 1) and a fluorescent probe (Taqman™). As observed in breast cancer tissues, complementation was also seen in the detection of circulating tumor cells derived from breast cancers. Again, mammaglobin PCR detected circulating tumor cells in a high percentage of blood samples, albeit at low levels, from metastatic breast cancer patients (20/32) when compared to the normal blood samples (Table 5) but several of the other genes tested to date further increased this detection rate. This included B726P, B305D, B311D, B533S and GABAπ. The detection level of mammaglobin in blood samples from metastatic breast cancer patients is higher than described previously (62 vs. 49%), despite testing smaller blood volumes, probably because of the use of epithelial marker-specific enrichment in our study. A combination of all the genes tested indicate that 27/32 samples were positive by one or more of these genes.

TABLE 5
Gene Complementation in Epithelial Cells Isolated from Blood of Normal Individuals
and Metastatic Breast Cancer Patients

| Sample ID | Mammaglobin | B305D | B311D | B533S | B726P | GABAπ | Combo |
|---|---|---|---|---|---|---|---|
| 2 | + | − | − | + | − | − | + |
| 3 | + | − | − | + | − | − | + |
| 5 | + | + | + | − | + | − | + |
| 6 | + | − | + | + | + | − | + |
| 8 | − | − | + | − | − | − | + |
| 9 | + | + | + | − | + | − | + |
| 10 | + | − | + | − | + | − | + |
| 11 | − | − | − | − | − | − | − |
| 12 | + | + | + | − | − | − | + |
| 13 | − | − | − | + | − | − | + |
| 15 | − | − | − | − | − | − | − |
| 18 | + | − | − | − | − | − | + |
| 19 | + | − | − | − | − | + | + |
| 21 | + | − | − | − | − | − | + |
| 22 | − | − | − | − | − | − | − |
| 23 | + | − | − | − | − | − | + |
| 24 | + | − | − | − | − | − | + |
| 25 | − | + | − | − | − | − | + |
| 26 | − | − | − | − | − | − | − |
| 29 | + | − | + | + | + | − | + |
| 31 | + | − | − | + | − | − | + |
| 32 | − | − | − | − | − | ± | ± |
| 33 | − | − | − | − | + | − | + |
| 34 | + | − | − | + | − | + | + |
| 35 | + | − | − | − | + | − | + |
| 36 | − | − | − | − | − | + | + |
| 37 | + | − | − | + | − | − | + |
| 38 | − | − | − | − | − | − | − |
| 40 | + | − | − | − | − | − | + |
| 41 | + | − | − | + | − | − | + |
| 42 | + | − | − | − | − | − | + |
| 43 | − | − | − | − | − | + | + |
| Donor 104 | − | − | − | − | − | + | + |
| Donor 348 | − | − | − | − | − | Nd | − |
| Donor 392 | − | − | − | − | − | Nd | − |
| Donor 408 | − | − | − | − | − | Nd | − |
| Donor 244 | − | − | − | − | − | − | − |
| Donor 355 | − | − | − | − | − | − | − |
| Donor 264 | − | − | − | − | − | − | − |
| Donor 232 | − | − | − | − | − | Nd | − |
| Donor 12 | − | − | − | − | − | − | − |
| Donor 415 | − | − | − | − | − | Nd | − |
| Donor 35 | − | − | − | − | − | − | − |
| Sensitivity | 20/32 | 4/32 | 7/32 | 9/32 | 7/32 | 4/32 | 27/32 |

In further studies, mammaglobin, GABAπ, B305D (C form) and B726P specific primers and specific Taqman probes were employed in different combinations to analyze their combined mRNA expression profile in breast metastases (B. met) and breast tumor (B. tumors) samples using real-time PCR. The forward and reverse primers and probes employed for mammaglobin, B305D (C form) and B726P are shown in Table 1. The forward primer and probe employed for GABAπ are shown in Table 1, with the reverse primer being as follows: TTCAAATATAAGTGAAGAAAAAATTAGTAGATCAA (SEQ ID NO:51). As shown below in Table 6, a combination of mammaglobin, GABAπ, B305D (C form) and B726P was found to detect 22/22 breast tumor samples, with an increase in expression being seen in 5 samples (indicated by ++).

TABLE 6
Real-time PCR Detection of Tumor Samples using Different Primer Combinations

| | | Mammaglobin + | Mammaglobin + | Mammaglobin + GABA + |
|---|---|---|---|---|
| Tumor sample | Mammaglobin | GABA | GABA + B305D | B305D +B726P |
| B. Met 316A | | + | + | + |
| B. Met 317A | + | + | + | + |
| B. Met 318A | | + | + | ++ |
| B. Met 595A | + | + | + | + |
| B. Met 611A | + | + | + | + |
| B. Met 612A | + | + | + | + |
| B. Met 614A | | + | + | + |
| B. Met 616A | | + | + | + |
| B. Met 618A | + | + | + | + |
| B. Met 620A | + | + | + | + |
| B. Met 621A | + | + | + | + |
| B. Met 624A | + | + | + | + |
| B. Met 625A | | | + | + |
| B. Met 627A | + | | + | + |
| B. Met 629A | | + | + | + |
| B. Met 631A | + | + | + | + |
| B. Tumor 154A | + | + | + | ++ |
| B. Tumor 155A | + | + | + | ++ |
| B. Tumor 81D | | | + | ++ |
| B. Tumor 209A | | + | + | + |
| B. Tumor 208A | | + | + | ++ |
| B. Tumor 10A | + | + | + | + |

The increase of message signals by the addition of specific primers was further demonstrated in a one plate experiment employing the four tumor samples B. met 316A, B. met 317A, B. tumor 81D and B. tumor 209A.

Expression of a combination of mammaglobin, GABAπ, B305D (C form) and B726P in a panel of breast tumor and normal tissue samples was also detected using real-time PCR with a SYBR Green detection system instead of the Taqman probe approach. The results obtained using this system are shown in FIG. 7.

### Example 7

### Quantitative PCR in Peripheral Blood of Breast Cancer Patients

The known genes evaluated in this study were mammaglobin and γ aminobutyrate type A receptor π subunit (GABAπ). In order to identify novel genes which are over-expressed in breast cancer we have used an improved version of the differential display RT-PCR (DDPCR) technique (Liang et al., Science 257:967-971 (1993); Mou et al., Biochem Biophy Res Commun. 199:564-569 (1994)); cDNA library extraction methods (Hara et al., Blood 84:189-199 (1994)) and PCR subtraction (Diatchenko et al., Proc Natl Acad Sci USA., 93:6025-6030 (1996); Yang et al., Nucleic Acids Res. 27:1517-23 (1999)).

Differential display resulted in the recovery of two cDNA fragments designated as B305D and B311D (Houghton et al., Cancer Res. 40:Abstract #217, 32-33, (1999). B511S and B533S are two cDNA fragments isolated using cDNA library subtraction approach (manuscript in preparation) while the B726P cDNA fragment was derived from PCR subtraction (Jiang et al., Proceedings of the Amer Assoc Cancer Res. 40:Abstract #216, 32 (1999); Xu et al., Proceedings of the Amer Assoc Cancer Res. 40:Abstract #2115, 319 (1999); and Molesh et al., Proceedings of Amer Assoc Cancer Res. 41:Abstract #4330, 681 (2000).

Three of the novel genes, B311D, B533S and B726P, showed identical breast tissue expression profile by quantitative PCR analysis. These genes were evaluated in quantitative PCR on two different panels consisting of (a) breast tumor, breast normal and normal tissues and (b) panel of breast tumor metastases (primarily lymph nodes). Primers and probes used are shown in Table 1. The data for panel (a) is shown in FIG. 2 for all three genes. Overall, the expression profiles are comparable and are in the same rank order, however, the levels of expression are considerably different. B311D in general was expressed at lower levels than B533S and both less than B726P but all three were restricted to breast tissue. All three sequences were used to search against the Genbank database. Both B311D and B533S sequences contain different repetitive sequences and an ORF has not been identified for either. B726P is a novel gene, with mRNA splicing yielding several different putative ORF's.

The quantitative PCR confirmed there was a differential mRNA expression between normal breast tissue and breast tumors, with approximately 50% of breast tumors overexpressed these genes. When tested on a panel of distant metastases derived from breast cancers all three genes reacted with 14/21 metastases and presented similar profiles (data not shown). Interestingly, when tested on a prostate cancer panel, all three genes identified the same 3/24 prostate tumors but at much lower expression levels than in breast. This group of genes exhibited increasing levels of expression as a function of pathological stage of the tumor as shown for B533 S.

More rigorous analysis of mammaglobin gene expression by microarray, and quantitative PCR showed expression at significant levels in skin and salivary gland and much lower levels in esophagus and trachea. B511S had a slightly different profile of reactivity on breast tumors and normal breast tissue when compared to mammaglobin, yet reacted with a similar subset of normal tissues as mammaglobin. Mammaglobin detected 14/24 of distant metastatic breast tumors, 31/42 breast tumors and exhibited ten-fold over-expression in tumors and metastases as compared to normal breast tissue. There was at least 300-fold over-expression of mammaglobin in normal breast tissue versus other negative normal tissues and tumors tested. B511S detected 33/42 breast tumors and 14/24 distant metastases. A combination of B511S with mammaglobin would be predicted to detect 38/42 breast tumors and 17/24 metastatic lesions. The quantitative level of expression of B511S and mammaglobin were also in similar ranges, in concordance with the microarray profiles observed for these two genes.

Certain genes complemented mammglobin's expression profile, i.e. were shown to express in tumors that mammaglobin did not. B305D was highly over-expressed in breast tumors, prostate tumors, normal prostate tissue and testis compared to normal tissues including normal breast tissue. Different splice variants of B305D were identified with the forms A and C being the most abundant. All forms tested had similar tissue profiles in quantitative PCR. The A and C forms contain ORF's of 320 and 385 aa, respectively. A known gene shown to be complementary with B305D, in breast tumors, was GABAπ. This tissue expression profile is in contrast to other GABA_{A} receptors that typically have appreciable expression in neuronal tissues. An additional observation was that tissue expression profiling of this gene showed it to be over-expressed in breast tumors in an inverse relationship to the B305D gene (Table 3). GABAπ detected 15/25 tumors and 6/21 metastases including 4 tumors and 5 metastases missed by mammaglobin. In contrast, B305D detected 13/25 breast tumors and 8/21 metastases again including 3 tumors and 2 metastases missed by mammaglobin. A combination of just B305D and the GABAπ would be predicted to identify 22/25 breast tumors and 14/21 metastases. This combination detected 20/21 of the breast metastases as well as 25/25 breast tumors that were evaluated on the same panels for all three genes. The one breast metastasis that was negative for these three genes was strongly positive for B726P.

The use of microarray analysis followed by quantitative PCR provided a methodology to accurately determine the expression of breast cancer genes both in breast tissues (tumor and normal) as well as in normal tissues and to assess their diagnostic and therapeutic potential. Five novel genes and two known genes were evaluated using these techniques. Three of these genes B311D, B533S and B726P exhibited concordant mRNA expression and collectively the data is consistent with coordinated expression of these three loci at the level of transcription control. All three genes showed differential expression in breast tumors versus normal breast tissue and the level of overexpression appeared related to the pathological stage of the tumor. In the case of mammaglobin, expression was found in other tissues apart from breast tissue. Expression was seen in skin, salivary gland and to a much lesser degree in trachea.

Expression of GABAπ in breast tumors was also a novel observation. While the expression of several genes complemented that seen with mammaglobin, two genes in particular, B305D and GABAπ added to the diagnostic sensitivity of mammaglobin detection. A combination of these three genes detected 45/46 (97.8%) breast tumors and metastases evaluated. Inclusion of B726P enabled the detection of all 25 of the breast tumors and 21 distant metastases.

### Example 8

### Enrichment of Circulating Breast Cancer Cells by Immunocapture

This example discloses the enhanced sensitivity achieved by use of the immunocapture cell capture methodology for enrichment of circulating breast cancer cells.

To evaluate the presence of circulating tumor cells an immunocapture method was adopted to first enrich for epithelial cells prior to RT-PCR analysis. Epithelial cells were enriched from blood samples with an immunomagnetic bead separation method (Dynal A. S, Oslo, Norway) utilizing magnetic beads coated with monoclonal antibodies specific for glycopolypeptide antigens on the surface of human epithelial cells. (Exemplary suitable cell-surface antigens are described, for example, in Momburg, F. et al., Cancer Res., 41:2883-91 (1997); Naume, B. et al., Journal of Hemotherapy. 6:103-113 (1997); Naume, B. et al., Int J Cancer. 78:556-60 (1998); Martin, V. M. et al., Exp Hematol., 26:252-64 (1998); Hildebrandt, M. et al., Exp Hematol. 25:57-65 (1997); Eaton, M. C. et al., Biotechniques 22:100-5 (1997); Brandt, B. et al., Clin Exp Metastases 14:399-408 (1996), each of which are incorporated herein by reference in their entirety. Cells isolated this way were lysed and the magnetic beads removed. The lysate was then processed for poly A⁺ mRNA isolation using magnetic beads (Dynabeads) coated with Oligo (dT)₂₅. After washing the beads in the kit buffer bead/polyA⁺RNA samples were finally suspended in 10 mM Tris HCl pH 8 and subjected to reverse transcription. The RNA was then subjected to Real time PCR using gene specific primers and probes with reaction conditions as outlined herein above. β-Actin content was also determined and used for normalization. Samples with gene of interest copies/ng P-actin greater than the mean of the normal samples+3 standard deviations were considered positive. Real time PCR on blood samples was performed exclusively using the Taqman™ procedure but extending to 50 cycles.

Mammaglobin mRNA using enrichment procedures was found to be detectable at much lower levels than when direct isolation was used. Whole blood samples from patients with metatastic breast cancer were subsequently treated with the immunomagnetic beads, polyA⁺ RNA was then isolated, cDNA made and run in quantitative PCR using two gene specific primers to mammaglobin and a fluorescent probe (Taqman™). As observed in breast cancer tissues, complementation was also seen in the detection of circulating tumor cells derived from breast cancers. Again, mammaglobin PCR detected circulating tumor cells in a high percentage of bloods, albeit at low levels, from metastatic breast cancer (20/32) when compared to the normal blood samples. Several of the other genes tested to date could further increase this detection rate; this includes B726P, B305D, B311D, B533S and GABAπ. A combination of all the genes tested indicates that 27/32 samples were positive by one or more of these genes.

### Example 9

### Multiplex Detection of Breast Tumors

Additional Multiplex Real-time PCR assays were established in order to simultaneously detect the expression of four breast cancer-specific genes: LipophilinB, Gaba (B899P), B305D-C and B726P. In contrast to detection approaches relying on expression analysis of single breast cancer-specific genes, this Multiplex assay was able to detect all breast tumor samples tested.

This Multiplex assay was designed to detect LipophilinB expression instead of Mammaglobin. Due to their similar expression profiles, LipophilinB can replace Mammaglobin in this Multiplex PCR assay for breast cancer detection. The assay was carried out as follows: LipophilinB, B899P (Gaba), B305D, and B726P specific primers, and specific Taqman probes, were used to analyze their combined mRNA expression profile in breast tumors. The primers and probes are shown below:

| | | |
|---|---|---|
| LipophilinB: | Forward Primer (SEQ ID NO:33): | 5′ TGCCCCTCCGGAAGCT. |
| | Reverse Primer (SEQ ID NO:34): | 5′ CGTTTCTGAAGGGACATCTGATC. |
| | Probe (SEQ ID NO:35) | (FAM-5′-3′-TAMRA): TTGCAGCCAAGTTAGGAGTGAAGAGATGCA. |
| GABA (B899P): | Forward Primer (SEQ ID NO:36): | 5′ AAGCCTCAGAGTCCTTCCAGTATG. |
| | Reverse Primer (SEQ ID NO:37): | 5′ TTCAAATATAAGTGAAGAAAAAATTAGTAGATCAA. |
| | Probe (SEQ ID NO:38) | (FAM-5′-3′-TAMRA):AATCCATTGTATCTTAGAACCGAGGGATTTGT |
| | | TTAGA. |
| B305D (C form): | Forward Primer (SEQ ID NO:39): | 5′ AAAGCAGATGGTGGTTGAGGTT. |
| | Reverse Primer (SEQ ID NO:40): | 5′ CCTGAGACCAAATGGCTTCTTC. |
| | Probe (SEQ ID NO:41) | (FAM-5′-3′-TAMRA) ATTCCATGCCGGCTGCTTCTTCTG. |
| B726P: | Forward Primer (SEQ ID NO:42): | 5′ TCTGGTTTTCTCATTCTTTATTCATTTATT. |
| | Reverse Primer (SEQ ID NO:43): | 5′ TGCCAAGGAGCGGATTATCT. |
| | Probe (SEQ ID NO:44) | (FAM-5′-3′-TAMRA): CAACCACGTGACAAACACTGGAATTACAGG. |
| Actin: | Forward Primer (SEQ ID NO:45): | 5′ ACTGGAACGGTGAAGGTGACA. |
| | Reverse Primer (SEQ ID NO 46): | 5′ CGGCCACATTGTGAACTTTG. |
| | Probe (SEQ ID NO:47): | (FAM-5′-3′-TAMRA): CAGTCGGTTGGAGCGAGCATCCC. |

The assay conditions were:

Tagman protocol (7700 Perkin Elmer):

In 25 μl final volume: 1×Buffer A, 5 mM MgCl, 0.2 mM dCTP, 0.2 mM dATP, 0.4 mM dUTP, 0.2 mM dGTP, 0.01 U/μl AmpErase UNG, 0.025 u/μl TaqGold, 8% (v/v) Glycerol, 0.05% (v/v) Gelatin, 0.01% (v/v) Tween20, 4 pmol of each gene specific Taqman probe (LipophilinB+Gaba+B305D+B726P), 100 nM of B726P-F+B726P-R, 300 nM of Gaba-R, and 50 nM of LipophilinB-F+LipophilinB-R+B305D-R+Gaba-R, template cDNA (originating from 0.02 μg polyA+RNA).

LipophilinB expression was detected in 14 out of 27 breast tumor samples. However, the Multiplex assay for LipophilinB, B899P, B305D-C and B726P detected an expression signal in 27 out of 27 tumors with the detection level above 10 mRNA copies/1000 pg actin in the majority of samples and above 100 mRNA copies/1000 pg actin in 5 out of the 27 samples tested (FIG. 8).

### Example 10

### Multiplex Detection Optimization

The Multiplex Real-time PCR assay described above was used to detect the expression of Mammaglobin (or LipophilinB), Gaba (B899P), B305D-C and B726P simultaneously. According to this Example, assay conditions and primer sequences were optimized to achieve parallel amplification of four PCR products with different lengths. Positive samples of this assay can be further characterized by gel electrophoresis and the expressed gene(s) of interest can be determined according to the detected amplicon size(s).

Mammaglobin (or LipophilinB), Gaba (B899P), B305D and B726P specific primers and specific Taqman probes were used to simultaneously detect their expression. The primers and probes used in this example are shown below.

| | | |
|---|---|---|
| Mammaglobin: | Forward Primer (SEQ ID NO:48): | 5′ TGCCATAGATGAATTGAAGGAATG. |
| | Reverse Primer (SEQ ID NO:49): | 5′ TGTCATATATTAATTGCATAAACACCTCA. |
| | Probe (SEQ ID NO:50): | (FAM-5′-3′-TAMRA): TCTTAACCAAACGGATGAAACTCTGAGCAATG. |
| GABA (B899P): | Forward Primer (SEQ ID NO:36): | 5′ AAGCCTCAGAGTCCTTCCAGTATG. |
| | Reverse Primer (SEQ ID NO:51): | 5′ ATCATTGAAAATTCAAATATAAGTGAAG. |
| | Probe (SEQ ID NO:38) | (FAM-5′-3′-TAMRA) AATCCATTGTATCTTAGAACCGAGGGATTTGTTT |
| | | AGA. |
| B305D (C form): | Forward Primer (SEQ ID NO:39): | 5′ AAAGCAGATGGTGGTTGAGGTT. |
| | Reverse Primer (SEQ ID NO:40): | 5′ CCTGAGACCAAATGGCTTCTTC. |
| | Probe (SEQ ID NO:41): | (FAM-5′-3′-TAMRA): ATTCCATGCCGGCTGCTTCTTCTG. |
| B726P: | Forward Primer (SEQ ID NO:52): | 5′ GTAGTTGTGCATTGAAATAATTATCATTAT. |
| | Reverse Primer (SEQ ID NO:43): | 5′ TGCCAAGGAGCGGATTATCT. |
| | Probe (SEQ ID NO:44) | (FAM-5′-3′-TAMRA): CAACCACGTGACAAACACTGGAATTACAGG. |

Primer locations and assay conditions were optimized to achieve parallel amplification of four PCR products with different sizes. The assay conditions were:

Tagman protocol (7700 Perkin Elmer):

In 25 μl final volume: 1×Buffer A, 5 mM MgCl, 0.2 mM dCTP, 0.2 mM dATP, 0.4 mM dUTP, 0.2 mM dGTP, 0.01 U/μl AmpErase UNG, 0.0375 U/μl TaqGold, 8% (v/v) Glycerol, 0.05% (v/v) Gelatin, 0.01% (v/v) Tween20, 4 pmol of each gene specific Taqman probe (Mammaglobin+Gaba+B305D+B726P), 300 nM of Gaba-R+Gaba-F, 100 nM of Mammaglobin-F+R; B726P-F+R, and 50 nM of B305D-F+R template cDNA (originating from 0.02 (μg polyA+RNA).

PCR protocol:

50° for 2′: x 1, 95° for 10′: X 1, and 95° for 15″/60° for 1′/68° for 1′: x 50.

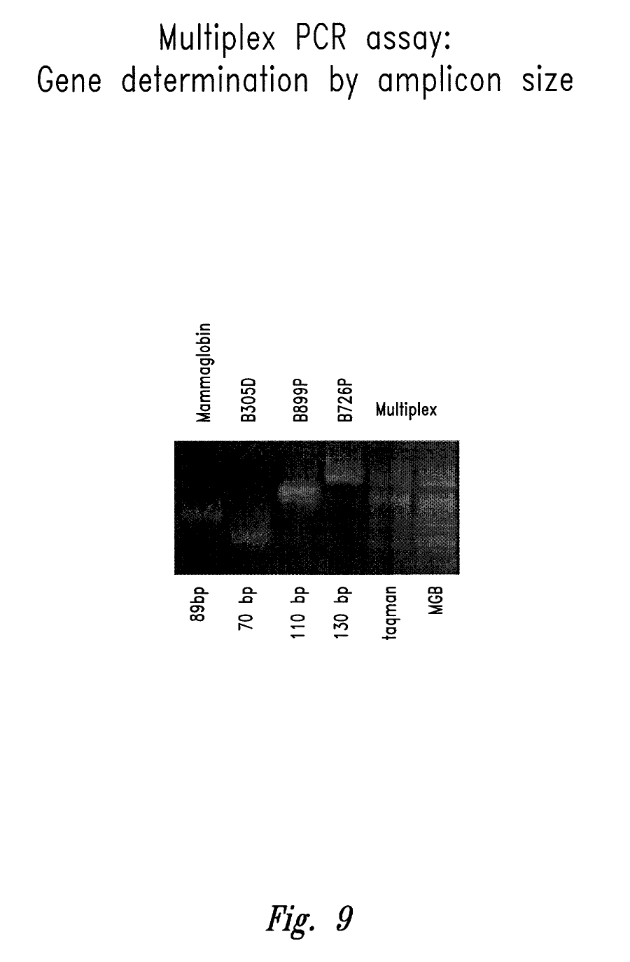

Since each primer set in the multiplex assay results in a band of unique length, expression signals of the four genes of interest can be measured individually by agarose gel analysis (see, FIG. 9), or the combined expression signal of all four genes can be measured in real-time on an ABI 7700 Prism sequence detection system (PE Biosystems, Foster City, Calif.). The expression of LipophilinB can also be detected instead of Mammaglobin. Although specific primers have been described herein, different primer sequences, different primer or probe labeling and different detection systems could be used to perform this multiplex assay. For example, a second fluorogenic reporter dye could be incorporated for parallel detection of a reference gene by real-time PCR. Or, for example a SYBR Green detection system could be used instead of the Taqman probe approach.

### Example 11

### Design Use of Genomic DNA-Excluding, Intron-Exon Border Spanning Primer Rairs for Breast Cancer Multiplex Assay

The Multiplex Real-time PCR assay described herein can detect the expression of Mammaglobin, Gaba (B899P), B305D-C and B726P simultaneously. The combined expression levels of these genes is measured in real-time on an ABI 7700 Prism sequence detection system (PE Biosystems, Foster City, Calif.). Individually expressed genes can also be identified due to different amplicon sizes via gel electrophoresis. In order to use this assay with samples derived from non-DNase treated RNAs (e.g. lymph node cDNA) and to avoid DNase-treatment for small RNA-samples (e.g. from blood specimens, tumor and lymph node aspirates), intron-spanning primer pairs have been designed to exclude the amplification of genomic DNA and therefore to eliminate nonspecific and false positive signals. False positive signal is caused by genomic DNA contamination in cDNA specimens. The optimized Multiplex assay described herein excludes the amplification of genomic DNA and allows specific detection of target gene expression without the necessity of prior DNase treatment of RNA samples. Moreover the genomic match and the location of the Intron-Exon border could be verified with these primer sets.

Mammaglobin, Gaba (B899P), B305D and B726P specific primers and specific Taqman probes were used to simultaneously detect their expression (Table 7).

Primer locations were optimized (Intron-Exon border spanning) to exclusively detect cDNA and to exclude genomic DNA from amplification. The identity of the expressed gene(s) was determined by gel electrophoresis.

TABLE 7
Intron-Exon border Spanning Primer and Probe Sequences
for Breast Tumor Multiples Assay

| | | | Taqman probe |
|---|---|---|---|
| Gene | Forward Primer | Reverse Primer | (FAM-5′-3′TAMRA) |
| Mammaglobin | tgccatagatgaattgaaggaatg | tgtcatatattaattgcataaacacctca | tcttaaccaaacggatgaaactctgagcaatg |
| | (SEQ ID NO:48) | (SEQ ID NO:49) | (SEQ ID NO:50) |
| B899P | aagcctcagagtccttccagtatg | ttcaaatataagtgaagaaaaaattagtagatcaa | aatccattgtatcttagaaccgagggatttgttt |
| | (SEQ ID NO:36) | (SEQ ID | (SEQ ID NO:62) |
| | | NO:37) | |
| B305D | aaagcagatggtggttgaggtt | cctgagaccaaatggcttcttc | attccatgccggctgcttcttctg (SEQ |
| | (SEQ ID NO:39) | (SEQ ID NO:40) | ID NO:41) |
| B726P | tctggttttctcattctttattcatttatt | tgccaaggagcggattatct | caaccacgtgacaaacactggaattacagg |
| | (SEQ ID NO:42) | (SEQ ID NO:43) | (SEQ ID NO:44) |
| Actin | actggaacggtgaaggtgaca | cggccacattgtgaactttg | cagtcggttggagcgagcatccc |
| | (SEQ ID NO:45 | (SEQ ID NO:46) | (SEQ ID NO:47) |
| B899P-INT | caattttggtggagaacccg | gctgtcggaggtatatggtg | catttcagagagtaacatggactacaca |
| | (SEQ ID NO:53) | (SEQ ID NO:54) | (SEQ ID NO:55) |
| B305D-INT | tctgataaaggccgtacaatg | tcacgacttgctgtttttgctc | atcaaaaaacaagcatggcctcacaccact |
| | (SEQ ID NO:56) | (SEQ ID NO:57) | (SEQ ID NO:58) |
| B726P-INT | gcaagtgccaatgatcagagg | atatagactcaggtatacacact | tcccatcagaatccaaacaagaggaagatg |
| | (SEQ ID NO:59) | (SEQ ID NO:60) | (SEQ ID NO:61) |

Primer locations and assay conditions were optimized to achieve parallel amplification of the four PCR products. The assay conditions were as follows:

Tagman protocol (7700 Perkin Elmer)

In 25 μl final volume: 1×Buffer A, 5 mM MgCl, 0.2 mM dCTP, 0.2 mM dATP, 0.4 mM dUTP, 0.2 mM dGTP, 0.01 U/AmpErase UNG, 8% (v/v) Glycerol, 0.05% (v/v) Gelatin, 0.01 % (v/v) Tween20, 4 pmol of each gene specific Taqman probe (Mammaglobin+B899P-INT+B305D-INT+B726P-INT), 300 nM of B305D-INT-F; B899P-INT-F, 100 nM of Mammaglobin-F+R; B726P-INT-F+R, 50 nM of B899P-INT-R; B305D-INT-R, template cDNA (originating from 0.02 μg polyA+RNA).

PCR Cycling Conditions

1 cycle at 50° C. for 2 minutes, 1 cycle at 95° C. for 10 minutes, 50 cycles of 95° C. for 1 minute and 68° C. for 1 minute.

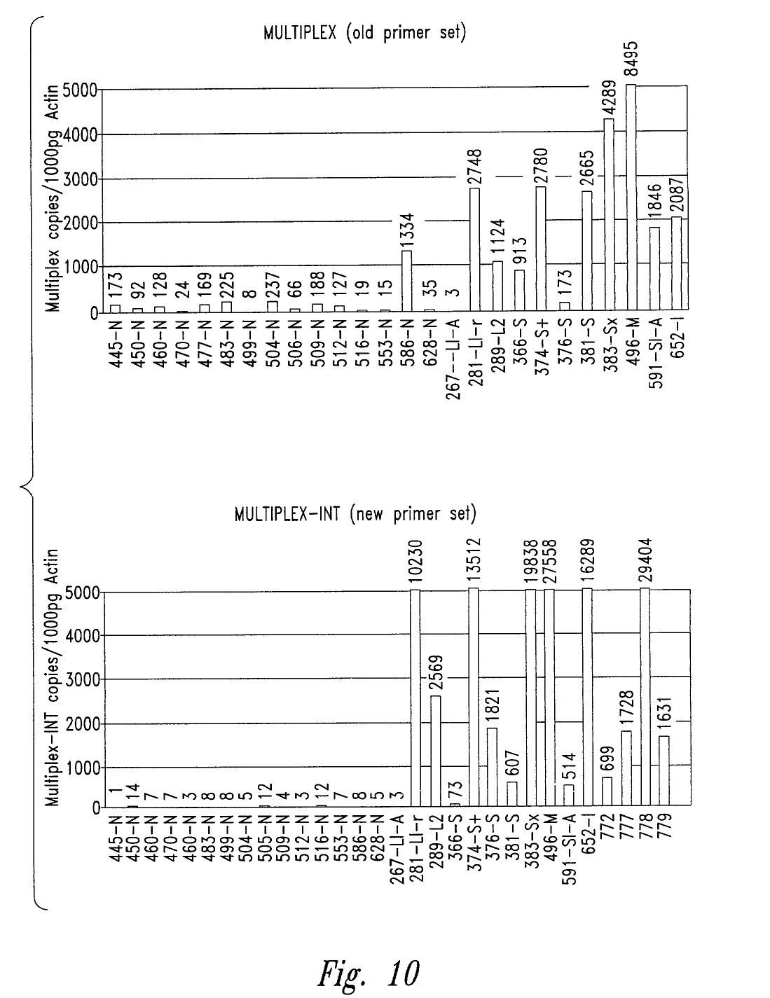

FIG. 10 shows a comparison of the multiplex assay using intron-exon border spanning primers (bottom panel) and the multiplex assay using non-optimized primers (top panel), to detect breast cancer cells in a panel of lymph node tissues. This experiment shows that reduction in background resulting from genomic DNA contamination in samples is achieved using the intron-exon spanning primers of the present invention.

### Example 12

### Multiplex Detection of Metastasized Breast Tumor Cells in Sentinel Lymph Node Biopsy Samples

Lymph node staging is important for determining appropriate adjuvant hormone and chemotherapy. In contrast to conventional axillary dissection a less invasive approach for staging of minimal residual disease is sentinel lymph node biopsy. Sentinel lymph node biopsy (SLNB) has the potential to improve detection of metastases and to provide prognostic values to lead to therapy with minimal morbidity associated with complete lymph node dissection. SLNB implements mapping of the one or two lymph nodes which primarily drain the tumor and therefore are most likely to harbor metastatic disease (the sentinel nodes). Routine pathological analysis of lymph nodes result in a high false-negative rate: one-third of women with pathologically negative lymph nodes develop recurrent disease [Bland: The Breast: Saunders 1991]. A more sensitive detection technique for tumor cells would be RT-PCR but its application is limited by lack of a single specific markers. The multimarker assay described above increases the likelihood of cancer detection across the population without producing false-positive results from normal lymph nodes.

As mentioned above, lymphatic afferents from a primary tumor drain into a single node, the sentinel lymph node, before drainage into the regional lymphatic basin occurs. Sentinel lymph nodes are located with dyes and/or radiolabelled colloid injected in the primary lesion site and sentinel lymph node biopsy allows pathological examination for micrometastatic deposits, staging of the axilla and therefore can avoid unnecessary axillary dissection. Nodal micrometastases can be located with staining (haematoxylin or eosin) or immunohistochemical analysis for cytokeratin proteins. Immunocytochemical staining techniques can produce frequent false-negative results by missing small metastatic foci due to inadequate sectioning of the node. Immunohistochemistry can result in false-positive results due to illegitimate expression of cytokeratins (reticulum cells) or in false-negative results when using the antibody Ber-Ep4 which corresponding antigen is not expressed on all tumor cells.

The multiplex assay described herein could provide a more sensitive detection tool for positive sentinel lymph nodes. Moreover the detection of breast cancer cells in bone marrow samples, peripheral blood and small needle aspiration samples is desirable for diagnosis and prognosis in breast cancer patients.

Twenty-two metastatic lymph node samples, in addition to 15 samples also previously analyzed and shown in FIG. 3A, were analyzed using the intron-exon border spanning multiplex PCR assay described herein. The results from this analysis are summarized in Table 8. Twenty-seven primary tumors were also analyzed and the results shown in Table 9. Twenty normal lymph node samples tested using this assay were all negative.

TABLE 8
Multiples Real-time PCR Analysis of 37 Metastatic Lymph Nodes

| breast | | | | | |
|---|---|---|---|---|---|
| metastatic | | | | | |
| lymph node | | | | | |
| samples | Mammaglobin | B305D | B899P | B726P | Multiplex |
| B. Met 317A | ++ | + | | + | +++ |
| B. Met 318A | | | ++ | | +++ |
| B. Met 595A | + | | | + | +++ |
| B. Met 611A | + | + | +++ | | ++ |
| B. Met 612A | ++ | ++ | | + | ++ |
| B. Met 614A | | ++ | | ++ | +++ |
| B. Met 616A | | | + | | ++ |
| B. Met 618A | +++ | + | | | +++ |
| B. Met 620A | ++ | ++ | | ++ | +++ |
| B. Met 621A | + | +++ | | + | +++ |
| B. Met 624A | | | ++ | | +++ |
| B. Met 625A | | ++ | | ++ | + |
| B. Met 627A | | + | | + | + |
| B. Met 629A | ++ | | | | +++ |
| B. Met 631A | + | | ++ | | + |
| 1255 | +++ | ++ | | ++ | ++ |
| 1257 | +++ | + | + | + | ++ |
| 769 | +++ | | | + | ++ |
| 1258 | ++ | + | + | | + |
| 1259 | | ++ | ++ | | +++ |
| 1250 | +++ | + | | + | +++ |
| 1726 | +++ | + | | + | +++ |
| 786 | +++ | + | + | | +++ |
| 281-LI-r | +++ | | | | +++ |
| 289-L2 | ++ | + | | | ++ |
| 366-S | + | | | | + |
| 374-S+ | +++ | ++ | | | +++ |
| 376-S | ++ | | | + | ++ |
| 381-S | + | + | | | + |
| 383-Sx | +++ | ++ | | | +++ |
| 496-M | +++ | ++ | | | +++ |
| 591-SI-A | + | + | | | + |
| 652-I | | + | ++ | | +++ |
| 772 | + | | | | + |
| 777 | + | + | | ++ | ++ |
| 778 | +++ | | | | +++ |
| 779 | + | | ++ | | ++ |

TABLE 9
Multiplex Real-time PCR Analysis of 27 Primary Breast Tumors

| breast primary | | | | | |
|---|---|---|---|---|---|
| tumor | | | | | |
| samples | Mammaglobin | B305D | B899P | B726P | Multiplex |
| T443 | + | ++ | | +++ | +++ |
| T457 | | + | + | | ++ |
| T395 | | | ++ | | ++ |
| T10A | + | +++ | | +++ | +++ |
| T446 | | + | | ++ | ++ |
| T11C | + | | +++ | | +++ |
| T23B | + | ++ | | | +++ |
| T207A | | ++ | | | + |
| T437 | + | + | | ++ | +++ |
| T391 | + | ++ | | +++ | +++ |
| T392 | + | + | | | ++ |
| TS76 | + | ++ | | | +++ |
| T483 | ++ | + | | | +++ |
| T81G | + | + | ++ | ++ | +++ |
| T430 | + | | ++ | | ++ |
| T465 | + | + | | + | ++ |
| TS80 | | | + | | + |
| T469 | + | | | + | +++ |
| T467 | + | | | ++ | +++ |
| T439 | | + | | | + |
| T387 | ++ | | + | + | ++ |
| T318 | | | + | | ++ |
| T154A | | | | + | + |
| T387A | +++ | | + | + | +++ |
| T155A | + | | ++ | + | + |
| T209A | | ++ | | | ++ |
| T208A | | + | | + | ++ |

### Example 13

### Detection of Circulating Breast Tumor Cells in Peripheral Blood and Bone Marrow Using a Multiplex Real-Time PCR Assay

Circulating breast tumor cells were detected in peripheral blood and bone marrow by either epithelial cell capture essentially as described in Example 8 and below or RosetteSep CD45 depletion followed by real-time multiplex PCR analysis. RosetteSep was used to enrich cells directly from a blood sample and consisted of a cocktail of tetrameric antibodies that target a variety of unwanted cells and crosslink them to glycophorin A on red blood cells (RBC) present in the sample, forming rosettes. When centrifuged over Ficoll, targeted cells pelleted along with the free RBC.

Three breast cancer cell lines were used for blood spiking experiments in different concentrations. All three cell lines (SKBR3, BT474, MB415) express Mammaglobin and therefore could be detected by single gene Mammaglobin RT-PCR.

Blood spiking and epithelial cell capture was carried out as follows: Peripheral blood from a normal, healthy donor drawn into EDTA containing vacutainers was used in 6 ml aliquots and spiked with 2, 20 or 200 cells/ml SKBR3, BT474 or MB415 cells. Mononuclear cells were isolated using Sigma Diagnostics Accuspin System-Histopaque-1077 (Sigma, St. Louis, Mo.) and resuspended in 5 ml PBS (/w 0.1% (w/v) BSA and 0.6% (g/L) Na-Citrate). For bone marrow samples and mononuclear cells, epithelial cell capture was performed using 100 ul of Dynabeads Epithelial Enrich (Dynal, Oslo, Norway) at 4° C. for 30 minutes. Dynabeads mRNA DIRECT Micro Kit was used for mRNA isolation and cDNA synthesis. CD45 depletion was carried out as follows: For blood spiking experiments and for patient samples, RosetteSep Antibody Cocktail (StemCell Technologies Inc., Vancouver, BC) was used to deplete CD45 positive cells. RNA was isolated using an mRNA isolation kit (F. Hoffmann-La Roche Ltd., Basel, Switzerland). The Multiplex real-time PCR assay was carried out as described in Example 11.

The results from the blood spiking experiments indicated that the RosetteSep CD45 depletion prior to multiplex real-time PCR was the most efficient and resulted in the most sensitive detection of breast tumor cells in circulation.

In addition, 42 peripheral blood patient samples from Senegal were tested using RosetteSep CD45 depletion followed by multiplex real-time PCR. 23 were found positive in the multiplex assay (Table 10). 35 bone marrow samples from breast cancer patients were tested using epithelial cell capture followed by multiplex real-time PCR. 23 were found to be positive (Table 11).

TABLE 10
ROSETTESEP CD45 DEPLETION AND MULTIPLEX REAL-TIME
RT-PCR ON BREAST CANCER PATIENT SAMPLES FROM SENEGAL

| | Triplicate | | |
|---|---|---|---|
| | Multiplex | Gene identification | |
| Sample | assay | via agarose gel | Pathology tissue biopsy |
| Tis04 | +−− | Gaba | ductal carcinoma, normal |
| | | | inflammatory |
| Tis10 | −−− | − | ductal carcinoma, invasive |
| Tis22 | +−− | Mammaglobin | inadequate, inflammatory |
| Tis25 | ++− | B305D + B726P | inadequate, inflammatory |
| Tis29 | −−− | − | lobular carcinoma, invasive |
| Tis24 | +++ | Mammaglobin | ductal carcinoma, invasive |
| Tis23 | −−− | − | normal inflammatory |
| Tis28 | −++ | Ambiguous | ductal carcinoma invasive |
| Tis02 | +++ | Mammaglobin | inadequate, inflammatory |
| Tis07 | −−+ | B305D | inadequate, inflammatory |
| Tis11 | +++ | Mammaglobin | Suspicious |
| Tis03 | −++ | Gaba | ductal carcinoma, invasive |
| Tis08 | +−− | B305D | ductal carcinoma, invasive |
| D157-Normal | −−− | − | − |
| D346-Normal | −−− | − | − |
| D151-Normal | −−− | − | − |
| Tis01 | −−− | − | ductal, invasive |
| Tis05 | −−− | − | ductal invasive |
| Tis06 | −−− | − | normal inflammatory |
| Tis09 | +−− | Gaba | inadequate, hyperplasia |
| Tis12 | −−− | − | inadequate, inflammatory |
| Tis13 | −−− | − | ductal invasive |
| Tis14 | +−− | B305D | inadequate, normal fibrous |
| | | | tissues |
| Tis15 | −−− | − | ductal carcinoma |
| Tis16 | +++ | Mammaglobin | ductal carcinoma invasive |
| Tis17 | −−− | − | normal inflammatory |
| Tis018 | −−− | − | ductal carcinoma |
| Tis26 | +++ | Gaba | ductal carcinoma invasive |
| Tis27 | ++− | Gaba + Mammaglobin | lobular carcinoma invasive |
| D140-Normal | −−− | − | − |
| Tis32 | ++− | Mammaglobin + Gaba | low grade ductal carcinoma |
| Tis34 | ++− | Mammaglobin + Gaba | +malignancy |
| Tis38 | −−− | − | +malignancy |
| Tis40 | ++− | B726P | +malignancy |
| Tis41 | ++− | B726P | +malignancy |
| Tis42 | −−− | − | ? |
| Tis43 | −−− | − | ? |
| Tis47 | +++ | Mammaglobin + | ? |
| | | Gaba | |
| D487 | −−− | − | Normal |
| D244 | −−− | − | Normal |
| D476 | −−− | − | Normal |
| D65 | −−− | − | Normal |
| Tis31 | +++ | Mammaglobin + | inadequate, inflammatory |
| | | Gaba | |
| Tis33 | −−− | − | +malignancy |
| Tis35 | −−+ | Gaba | +malignancy |
| Tis36 | ++− | Gaba | +malignancy |
| Tis37 | +++ | Mammaglobin + | +malignancy |
| | | Gaba | |
| Tis39 | ++− | Ambiguous | inadequate |
| Tis44 | +−− | Ambiguous | ? |
| Tis46 | +−− | Ambiguous | ? |
| D151-Normal | −−− | − | − |
| D214-Normal | −−− | − | − |
| D100-Normal | −−− | − | − |

TABLE 11
EPITHELIAL CELL CAPTURE AND MULTIPLEX
REAL-TIME PCR ASSAY ON BONE MARROW
SAMPLES FROM BREAST CANCER PATIENTS

| Sample | Tumor | | Bone Marrow* | | |
|---|---|---|---|---|---|
| ID | Status | Tumor Type | Right | Left | Blood |
| 4 | T1bN0 | Invasive | Mammaglobin | — | Nd |
| | | Lobular | | | |
| 5 | T2N1b | Invasive | — | — | Nd |
| | | Lobular | | | |
| 6 | T2N1b | Invasive | — | — | Nd |
| | | Ductal | | | |
| 7 | T2N0 | Invasive | Mammaglobin | GABA/B726P | Nd |
| | | Ductal | | | |
| 8 | T3N1 | Invasive | — | GABA | Nd |
| | | Lobular | | | |
| 9 | T2N0 | Invasive | Mammaglobin | Mammaglobin | Nd |
| | | Ductal | | | |
| 10 | T1cN0 | Invasive | — | — | Nd |
| | | Ductal | | | |
| 11 | T2N1b | Invasive | B726P | — | Nd |
| | | Ductal | | | |
| 12 | T1cN0 | Invasive | — | — | B726P |
| | | Ductal | | | |
| 13 | T1cNo | Invasive | GABA | Mammaglobin | — |
| | | Ductal | | | |
| 14 | T1bN0 | Colloid | — | — | — |
| 15 | T2N0 | Colloid, | — | B726P | — |
| | | ductal | | | |
| 16 | T1cN0 | DCIS, | GABA | — | Mamma- |
| | | Invasive | | | globin/B726P |
| | | ductal | | | |
| 17 | T1cN0 | Invasive | — | Nd | — |
| | | Ductal | | | |
| 18 | T1cN1b, c | Invasive | — | — | — |
| | | Ductal | | | |
| 19 | T1bN0 | Tubular | B305D | Mammaglobin | — |
| | | Carcinoma | | | |
| 20 | T1bN0 | Invasive | Mammaglobin | — | B726P |
| | | Ductal | | | |
| 21 | T1cN0 | Invasive | — | GABA | — |
| | | Ductal | | | |
| 22 | TxN1 | Invasive | — | — | — |
| | | Ductal | | | |
| 23 | T1cN0 | Invasive | Mammaglobin | Nd | — |
| | | Ductal | | | |
| 24 | T2, T1bN0 | Invasive | GABA | B726P | — |
| | | Ductal | | | |
| 25 | T1bN0 | Invasive | Mammaglobin/ | — | — |
| | | Ductal | B305D | | |
| 26 | T1cN0 | Invasive | GABA | — | — |
| | | Ductal | | | |
| 27 | T1cN0 | Invasive | — | GABA | B305D |
| | | Ductal | | | |
| 28 | ?N0 | Invasive | Mammaglobin | — | — |
| | | Ductal, | | | |
| | | tubular | | | |
| 29 | T1N0 | Invasive | B305D/GABA | B305D/GABA | Nd |
| | | Ductal | | | |
| 30 | T2N1b | Invasive | Nd | B305D | — |
| | | Lobular | | | |
| 31 | T2N0 | Invasive | — | — | — |
| | | Ductal | | | |
| 32 | T1cN0 | Invasive | — | — | — |
| | | Lobular, | | | |
| | | ductal | | | |
| 33 | T1cN0 | Invasive | — | — | — |
| | | Lobular | | | |
| 34 | T1cN0 | Invasive | — | GABA | — |
| | | Ductal | | | |
| 35 | T3N0 | Invasive | GABA | — | — |
| | | Ductal | | | |
| 36 | T2N1b | Invasive | GABA | — | — |
| | | Lobular | | | |
| 37 | T1bN1a | Invasive | — | — | Mammaglobin |
| | | Ductal | | | |
| 38 | T1aN0 | Invasive | Mammaglobin | — | — |
| | | Ductal | | | |

*Gene identification by gel analysis
Nd: not determined

From the foregoing it will be appreciated that, although specific embodiments of the invention have been described herein for purposes of illustration, various modifications may be made without deviating from the spirit and scope of the invention. Accordingly, the invention is not limited except as by the appended claims.

We claim:

1. A method for identifying one or more tissue-specific polynucleotides, said method comprising the steps of:
(a) performing a genetic subtraction to identify a pool of polynucleotides from a tissue of interest;
(b) performing a DNA microarray analysis to identify a first subset of said pool of polynucleotides of interest wherein each member polynucleotide of said first subset is at least two-fold over-expressed in said tissue of interest as compared to a control tissue; and
(c) performing a quantitative polymerase chain reaction (PCR) analysis on polynucleotides within said first subset to identify a second subset of polynucleotides that are at least two-fold over-expressed as compared to said control tissue;
wherein a polynucleotide is identified as tissue-specific if it is at least two-fold over-expressed by both microarray and quantitative PCR analyses.

2. The method of claim 1 wherein said genetic subtraction is selected from the group consisting of differential display and cDNA subtraction.

3. A method for identifying a subset of polynucleotides showing complementary tissue-specific expression profiles in a tissue of interest, said method comprising the steps of:
(a) performing a first expression analysis selected from the group consisting of DNA microarray and quantitative PCR to identify a first polynucleotide that is at least two-fold over-expressed in a first tissue sample of interest obtained from a first patient but not over-expressed in a second tissue sample of interest as compared to a control tissue; and
(b) performing a second expression analysis selected from the group consisting of DNA microarray and quantitative PCR to identify a second polynucleotide that is at least two-fold over-expressed in a second tissue sample of interest obtained from a second patient but not over-expressed in a first tissue sample of interest as compared to said control tissue;
wherein the first tissue sample and said second tissue sample are of the same tissue type, and wherein over-expression of said first polynucleotide in only said first tissue samples of interest and over-expression of said second polynucleotide in only said second tissue sample of interest indicates complementary tissue-specific expression of said first polynucleotide and said second polynucleotide.

4. A method for determining the presence of a cancer cell in a patient, said method comprising the steps of:
(a) obtaining a biological sample from said patient;
(b) contacting said biological sample with a first oligonucleotide that hybridizes to a first polynucleotide said first polynucleotide selected from the group consisting of polynucleotides depicted in SEQ ID NO:73, SEQ ID NO:74 and SEQ ID NO:76;
(c) contacting said biological sample with a second oligonucleotide that hybridizes to a second polynucleotide selected from the group consisting of SEQ ID NO: 1, 3, 5-7, 11, 13, 15, 17, 19-24, 30, 32, and 75;
(d) detecting in said sample an amount of a polynucleotide that hybridizes to at least one of said oligonucleotides; and
(e) comparing the amount of the polynucleotide that hybridizes to said oligonucleotide to a predetermined cut-off value, and therefrom determining the presence or absence of a cancer in the patient.

5. A method for determining the presence or absence of a cancer in a patient, said method comprising the steps of:
(a) obtaining a biological sample from said patient;
(b) contacting said biological sample with a first oligonucleotide that hybridizes to a first polynucleotide selected from the group consisting of polynucleotides depicted in SEQ ID NO:73, SEQ ID NO:74 and SEQ ID NO:76;
(c) contacting said biological sample with a second oligonucleotide that hybridizes to a second polynucleotide as depicted in SEQ ID NO:75;
(d) contacting said biological sample with a third oligonucleotide that hybridizes to a third polynucleotide selected from the group consisting of polynucleotides depicted in SEQ ID NO:5, SEQ ID NO:6 and SEQ ID NO:7;
(e) contacting said biological sample with a fourth oligonucleotide that hybridizes to a fourth polynucleotide selected from the group consisting of polynucleotides depicted in SEQ ID NO:13, SEQ ID NO:15, SEQ ID NO:17, SEQ ID NO:19, SEQ ID NO:20, SEQ ID NO:21, SEQ ID NO:22, SEQ ID NO:23 and SEQ ID NO:24;
(f) detecting in said biological sample an amount of a polynucleotide that hybridizes to at least one of said oligonucleotides; and
(g) comparing the amount of polynucleotide that hybridizes to the oligonucleotide to a predetermined cut-off value, and therefrom determining the presence or absence of a cancer in the patient.

6. A method for determining the presence or absence of a cancer in a patient, said method comprising the steps of:
(a) obtaining a biological sample from said patient;
(b) contacting said biological sample with an oligonucleotide that hybridizes to a tissue-specific polynucleotide;
(c) detecting in the sample a level of a polynucleotide that hybridizes to the oligonucleotide; and
(d) comparing the level of polynucleotide that hybridizes to the oligonucleotide with a predetermined cut-off value, and therefrom determining the presence or absence of a cancer in the patient.

7. A method for monitoring the progression of a cancer in a patient, said method comprising the steps of:
(a) obtaining a first biological sample from said patient;
(b) contacting said biological sample with an oligonucleotide that hybridizes to a polynucleotide that encodes a breast tumor protein;
(c) detecting in the sample an amount of said polynucleotide that hybridizes to said oligonucleotide;
(d) repeating steps (b) and (c) using a second biological sample obtained from said patient at a subsequent point in time; and
(e) comparing the amount of polynucleotide detected in step (d) with the amount detected in step (c) and therefrom monitoring the progression of the cancer in the patient.

8. The method any one of claim 6 and claim 7 wherein said polynucleotide encodes a breast tumor protein selected from the group consisting of mammaglobin, lipophilin B, GABAπ (B899P), B726P, B511S, B533S, B305D and B311D.

9. A method for detecting the presence of a cancer cell in a patient, said method comprising the steps of:
(a) obtaining a biological sample from said patient;
(b) contacting said biological sample with a first oligonucleotide that hybridizes to a first polynucleotide selected from the group consisting of mammaglobin and lipophilin B;
(c) contacting said biological sample with a second oligonucleotide that hybridizes to a second polynucleotide sequence selected from the group consisting of GABAπ (B899P), B726P, B511S, B533S, B305D and B311D;
(d) detecting in said biological sample an amount of a polynucleotide that hybridizes to at least one of the oligonucleotides; and
(e) comparing the amount of polynucleotide that hybridizes to the oligonucleotide to a predetermined cut-off value, and therefrom determining the presence or absence of a cancer in the patient.

10. A method for determining the presence of a cancer cell in a patient, said method comprising the steps of:
(a) obtaining a biological sample from said patient;
(b) contacting said biological sample with a first oligonucleotide that hybridizes to a first polynucleotide selected from the group consisting of a polynucleotide depicted in SEQ ID NO:73 and SEQ ID NO:74 or complement thereof;
(c) contacting said biological sample with a second oligonucleotide that hybridizes to a second polynucleotide depicted in SEQ ID NO:75 or complement thereof;
(d) contacting said biological sample with a third oligonucleotide that hybridizes to a third polynucleotide selected from the group consisting of a polynucleotide depicted in SEQ ID NO:1, SEQ ID NO:3, SEQ ID NO:5, SEQ ID NO:6 and SEQ ID NO:7 or complement thereof;
(e) contacting said biological sample with a fourth oligonucleotide that hybridizes to a fourth polynucleotide selected from the group consisting of a polynucleotide depicted in SEQ ID NO: 11 or complement thereof;
(f) contacting said biological sample with a fifth oligonucleotide that hybridizes to a fifth polynucleotide selected from the group consisting of a polynucleotide depicted in SEQ ID NO:13, 15 and 17 or complement thereof;
(g) contacting said biological sample with a sixth oligonucleotide that hybridizes to a sixth polynucleotide selected from the group consisting of a polynucleotide depicted in SEQ ID NO:19, SEQ ID NO:20, SEQ ID NO:21, SEQ ID NO:22, SEQ ID NO:23 and SEQ ID NO:24 or complement thereof;
(h) contacting said biological sample with a seventh oligonucleotide that hybridizes to a seventh polynucleotide depicted in SEQ ID NO:30 or complement thereof;
(i) contacting said biological sample with an eighth oligonucleotide that hybridizes to an eighth polynucleotide depicted in SEQ ID NO:32 or complement thereof;
(j) contacting said biological sample with a ninth oligonucleotide that hybridizes to a polynucleotide depicted in SEQ ID NO:76 or complement thereof;
(k) detecting in said biological sample a hybridized oligonucleotide of any one of steps (b) through (j) and comparing the amount of polynucleotide that hybridizes to the oligonucleotide to a predetermined cut-off value,
wherein the presence of a hybridized oligonucleotide in any one of steps (b) through (j) in excess of the pre-determined cut-off value indicates the presence of a cancer cell in the biological sample of said patient.

11. A method for determining the presence of a cancer cell in a patient, said method comprising the steps of:
(a) obtaining a biological sample from said patient;
(b) contacting said biological sample with a first oligonucleotide and a second oligonucleotide;
i. wherein said first oligonucleotide and said second oligonucleotide hybridize to a first polynucleotide and a second polynucleotide, respectively;
ii. wherein said first polynucleotide and said second polynucleotide are selected from the group consisting of polynucleotides deptided in SEQ ID NO:73, SEQ ID NO:74, SEQ ID NO:75, SEQ ID NO:1, SEQ ID NO:3, SEQ ID NO:5, SEQ ID NO:6, SEQ ID NO:7, SEQ ID NO:1, SEQ ID NO:13, SEQ ID NO:15, SEQ ID NO:17, SEQ ID NO:19, SEQ ID NO:20, SEQ ID NO:21, SEQ ID NO:22, SEQ ID NO:23, SEQ ID NO:24, SEQ ID NO:30, SEQ ID NO:32, and SEQ ID NO:76; and
iii. wherein said first polynucleotide is unrelated in nucleotide sequence to said second polynucleotide;
(c) detecting in said biological sample said hybridized first oligonucleotide and said hybridized second hybridized oligonucleotide; and
(d) comparing the amount of said hybridized first oligonucleotide and said hybridized second hybridized oligonucleotide to a predetermined cut-off value;
wherein an amount of said hybridized first oligonucleotide or said hybridized second oligonucleotide in excess of the pre-determined cut-off value indicates the presence of a cancer cell in the biological sample of said patient.

12. A method for determining the presence or absence of a cancer cell in a patient, said method comprising the steps of:
(a) obtaining a biological sample from said patient;
(b) contacting said biological sample with a first oligonucleotide and a second oligonucleotide;
i. wherein said first oligonucleotide and said second oligonucleotide hybridize to a first polynucleotide and a second polynucleotide, respectively;
ii. wherein said first polynucleotide and said second polynucleotide are both tissue-specific polynucleotides of the cancer cell to be detected; and
iii. wherein said first polynucleotide is unrelated in nucleotide sequence to said second polynucleotide;
(c) detecting in said biological sample said first hybridized oligonucleotide and said second hybridized oligonucleotide; and
(d) comparing the amount of polynucleotide that hybridizes to the oligonucleotide to a predetermined cut-off value, wherein the presence of a hybridized first oligonucleotide or a hybridized second oligonucleotide in excess of the pre-determined cut-off value indicates the presence of a cancer cell in the biological sample of said patient.

13. A method for detecting the presence of a cancer cell in a patient, said method comprising the steps of:
(a) obtaining a biological sample from said patient;
(b) contacting said biological sample with a first oligonucleotide pair said first pair comprising a first oligonucleotide and a second oligonucleotide wherein said first oligonucleotide and said second oligonucleotide hybridize to a first polynucleotide and the complement thereof, respectively;
(c) contacting said biological sample with a second oligonucleotide pair said second pair comprising a third oligonucleotide and a fourth oligonucleotide wherein said third and said fourth oligonucleotide hybridize to a second polynucleotide and the complement thereof, respectively, and wherein said first polynucleotide is unrelated in nucleotide sequence to said second polynucleotide;
(d) amplifying said first polynucleotide and said second polynucleotide; and
(e) detecting said amplified first polynucleotide and said amplified second polynucleotide;
wherein the presence of said amplified first polynucleotide or said amplified second polynucleotide indicates the presence of a cancer cell in said patient.

14. The method of any one of claims 4-7 and 9-13 wherein said biological sample is selected from the group consisting of blood, serum, lymph node, bone marrow, sputum, urine and tumor biopsy sample.

15. The method of claim 14 wherein said biological sample is selected from the group consisting of blood, a lymph node and bone marrow.

16. The method of claim 15 wherein said lymph node is a sentinel lymph node.

17. The method of any one of claims 4-7 and 9-13 wherein said cancer is selected from the group consisting of prostate cancer, breast cancer, colon cancer, ovarian cancer, lung cancer head & neck cancer, lymphoma, leukemia, melanoma, liver cancer, gastric cancer, kidney cancer, bladder cancer, pancreatic cancer and endometrial cancer.

18. The method of any one of claims 12 and 13 wherein said first polynucleotide and said second polynucleotide are selected from the group consisting of mammaglobin, lipophilin B, GABAπ (B899P), B726P, B511S, B533S, B305D and B311D.

19. The method of any one of claims 12 and 13 wherein said first polynucleotide and said second polynucleotide are selected from the group consisting of polynucleotide depicted in SEQ ID NO:73, SEQ ID NO:74, SEQ ID NO:75, SEQ ID NO:1, SEQ ID NO:3, SEQ ID NO:5, SEQ ID NO:6, SEQ ID NO:7, SEQ ID NO:11, SEQ ID NO:13, SEQ ID NO:15, SEQ ID NO:17, SEQ ID NO:19, SEQ ID NO:20, SEQ ID NO:21, SEQ ID NO:22, SEQ ID NO:23, SEQ ID NO:24, SEQ ID NO:30, SEQ ID NO:32, and SEQ ID NO:76.

20. The method of any one of claims 12 and 13 wherein said oligonucleotides are selected from the group consisting of oligonucleotides depicted in SEQ ID NOs:33-35 and 63-72.

21. The method of any one of claims 12 and 13 wherein the step of detection of said first amplified polynucleotide and said second polynucleotide comprises a step selected from the group consisting of detecting a radiolabel and detecting a fluorophore.

22. The method of any one of claims 4-7 and 9-13 wherein said step of detection comprises a step of fractionation.

23. The method of any one of claims 12 and 13 wherein said first and said oligonucleotides are intron spanning oligonucleotides.

24. The method of claim 23 wherein said intron spanning oligonucleotides are selected from the group consisting of oligonucleotides depicted in SEQ ID NO:36-62.

25. The method of claim 13 wherein detection of said amplified first or said second polynucleotide comprises contacting said amplified first or said second polynucleotide with a labeled oligonucleotide probe that hybridizes, under moderately stringent conditions, to said first or said second polynucleotide.

26. The method of claim 13 wherein said labeled oligonucleotide probe comprises a detectable moiety selected from the group consisting of a radiolabel and a fluorophore.

27. The method of any one of claims 4-7 and 9-13 further comprising a step of enriching said cancer cell from said biological sample prior to hybridizing said oligonucleotide primer(s).

28. The method of claim 27 wherein said step of enriching said cancer cell from said biological sample is achieved by a methodology selected from the group consisting of cell capture and cell depletion.

29. The method of claim 28 wherein cell capture is achieved by immunocapture, said immunocapture comprising the steps of:
(a) adsorbing an antibody to the surface of said cancer cells; and
(b) separating said antibody adsorbed cancer cells from the remainder of said biological sample.

30. The method of claim 29 wherein said antibody is directed to an antigen selected from the group consisting of CD2, CD3, CD4, CD5, CD8, CD10, CD11b, CD14, CD15, CD16, CD19, CD20, CD24, CD25, CD29, CD33, CD34, CD36, CD38, CD41, CD45, CD45RA, CD45RO, CD56, CD66B, CD66e, HLA-DR, IgE and TCRαβ.

31. The method of claim 29 wherein said antibody is directed to a breast tumor antigen.

32. The method of any one of claims 29-31 wherein said antibody is a monoclonal antibody.

33. The method of claim 29 wherein said antibody is conjugated to magnetic beads.

34. The method of claim 29 wherein said antibody is formulated in a tetrameric antibody complex.

35. The method of claim 28 wherein cell depletion is achieved by a method comprising the steps of:
(a) cross-linking red cells and white cells, and
(b) fractionating said cross-linked red and white cells from the remainder of said biological sample.

36. The method of claim 13 wherein said step of amplifying is achieved by a polynucleotide amplification methodology selected from the group consisting of reverse transcription polymerase chain reaction (RT-PCR), inverse PCR, RACE, ligase chain reaction (LCR), Qbeta Replicase, isothermal amplification, strand displacement amplification (SDA), rolling chain reaction (RCR), cyclic probe reaction (CPR), transcription-based amplification systems (TAS), nucleic acid sequence based amplification (NASBA) and 3SR.

37. A composition for detecting a cancer cell in a biological sample of a patient, said composition comprising:
(a) a first oligonucleotide; and
(b) a second oligonucleotide;
wherein said first oligonucleotide and said second oligonucleotide hybridize to a first polynucleotide and to a second polynucleotide, respectively; wherein said first polynucleotide is unrelated in nucleotide sequence from said second polynucleotide; and
wherein said first polynucleotide and said second polynucleotide are tissue-specific polynucleotides of the cancer cell to be detected.

38. The composition of claim 37 wherein said first polynucleotide and said second polynucleotide are complementary tissue-specific polynucleotides of the tissue-type of said cancer cell.

39. The composition of any one of claim 37 and claim 38 wherein said first polynucleotide and said second polynucleotide are selected from the group consisting of the polynucleotides depicted in SEQ ID NO:73, SEQ ID NO:74, SEQ ID NO:75, SEQ ID NO:1, SEQ ID NO:3, SEQ ID NO:5, SEQ ID NO:6, SEQ ID NO:7, SEQ ID NO:11, SEQ ID NO:13, SEQ ID NO:15, SEQ ID NO:17, SEQ ID NO:19, SEQ ID NO:20, SEQ ID NO:21, SEQ ID NO:22, SEQ ID NO:23, SEQ ID NO:24, SEQ ID NO:30, SEQ ID NO:32, and SEQ ID NO:76.

40. The composition of any one of claim 37 and claim 38 wherein said oligonucleotides are selected from the group consisting of oligonucleotides as disclosed in SEQ ID NO: 33-72.

41. A composition for detecting a cancer cell in a biological sample of a patient, said composition comprising:
(a) a first oligonucleotide pair; and
(b) a second oligonucleotide pair;
wherein said first oligonucleotide pair and said second oligonucleotide pair hybridize to a first polynucleotide (or complement thereof) and to a second polynucleotide (or complement thereof), respectively; wherein said first polynucleotide is unrelated in nucleotide sequence from said second polynucleotide; and wherein said first polynucleotide and said second polynucleotide are tissue-specific polynucleotides of the cancer cell to be detected.

42. The composition of claim 41 wherein said first polynucleotide and said second polynucleotide are complementary tissue-specific polynucleotides of the tissue-type of said cancer cell.

43. The composition of any one of claim 41 and claim 42 wherein said first polynucleotide and said second polynucleotide are selected from the group consisting of the polynucleotides depicted in SEQ ID NO:73, SEQ ID NO:74, SEQ ID NO:75, SEQ ID NO:1, SEQ ID NO:3, SEQ ID NO:5, SEQ ID NO:6, SEQ ID NO:7, SEQ ID NO:11, SEQ ID NO:13, SEQ ID NO:15, SEQ ID NO:17, SEQ ID NO:19, SEQ ID NO:20, SEQ ID NO:21, SEQ ID NO:22, SEQ ID NO:23, SEQ ID NO:24, SEQ ID NO:30, SEQ ID NO:32, and SEQ ID NO:76.

44. The composition of any one of claim 41 and claim 42 wherein said oligonucleotides are selected from the group consisting of oligonucleotides as disclosed in SEQ ID NO: 33-72.

45. A composition comprising an oligonucleotide primer or probe of between 15 and 100 nucleotides that comprises an oligonucleotide selected from the group consisting of oligonucleotides depicted in SEQ ID NOs:33-72.

46. The composition of claim 45 comprising an oligonucleotide primer or probe selected from the group consisting of oligonucleotides depicted in SEQ ID NOs:33-72.
